(19) United States
(12) Patent Application Publication
(10) Pub. No.: US 2025/0163035 A1
(43) Pub. Date: May 22, 2025

(54) MATERIALS FOR ORGANIC ELECTROLUMINESCENT DEVICES

(71) Applicant: Merck Patent GmbH, Darmstadt (DE)

(72) Inventors: Amir Hossain PARHAM, Darmstadt (DE); Klaus OMOREGBEE, Darmstadt (DE)

(21) Appl. No.: 18/836,787

(22) PCT Filed: Feb. 6, 2023
(86) PCT No.: PCT/EP2023/052756

§ 371 Date: Aug. 8, 2024

(30) Foreign Application Priority Data
Feb. 9, 2022 (EP) 22155956.0

(51) Int. Cl. C07D 413/04; C07B 59/00; C07D 409/14; H10K 85/60

(52) U.S. Cl. CPC C07D 413/04; C07B 59/004; C07D 409/14; H10K 85/622; H10K 85/6572; H10K 85/6574; H10K 85/6576; C07B 2200/05

(57) ABSTRACT

The present invention relates to compounds that are suitable for use in electronic devices, and to electronic devices, particularly organic electroluminescent devices, containing these compounds.

The present invention describes compounds, especially for use in electronic devices. The invention further relates to a process for preparing the compounds of the invention and to electronic devices comprising these compounds.

The structure of organic electroluminescent devices in which organic semiconductors are used as functional materials is described, for example, in U.S. Pat. Nos. 4,539,507, 5,151,629, EP 0676461, WO 98/27136 and WO 2010/151006 A1. Emitting materials used are frequently organometallic complexes which exhibit phosphorescence. For quantum-mechanical reasons, up to four times the energy efficiency and power efficiency is possible using organometallic compounds as phosphorescent emitters. In general terms, there is still a need for improvement in electroluminescent devices, especially also in electroluminescent devices which exhibit phosphorescence, for example with regard to efficiency, operating voltage and lifetime. Also known are organic electroluminescent devices comprising fluorescent emitters or emitters that exhibit TADF (thermally activated delayed fluorescence).

According to the prior art, dibenzothiophene dioxides according to CN110790756 or KR2015/0031396, and also according to WO2019/016430, are among the matrix materials used for phosphorescent emitters or as electron transport materials.

The properties of organic electroluminescent devices are not only determined by the emitters used. Also of particular significance here are especially the other materials used, such as matrix materials, hole blocker materials, electron transport materials, hole transport materials and electron or exciton blocker materials. Improvements to these materials can lead to distinct improvements to electroluminescent devices.

In general terms, in the case of these materials, for example for use as matrix materials, hole transport materials or electron transport materials, there is still a need for improvement, particularly in relation to efficiency and operating voltage, but also to the lifetime of the device. Moreover, OLEDs containing the compounds are supposed to have high color purity.

It is an object of the present invention to provide compounds which are suitable for use as matrix materials or charge transport materials in an organic electronic device, especially in an organic electroluminescent device, and which lead to good device properties when used in this device, and to provide the corresponding electronic device.

It is a particular object of the present invention to provide compounds which lead to a high lifetime, good efficiency and low operating voltage.

A further object of the present invention can be considered that of providing compounds suitable for use in phosphorescent or fluorescent electroluminescent devices, especially as a matrix material. A particular object of the present invention is that of providing matrix materials suitable for red-, yellow- and green-phosphorescing electroluminescent devices.

It has been found that, surprisingly, these objects are achieved by particular compounds that are described in detail hereinafter, and these have lower operating voltage and higher efficiency with similar lifetime compared to materials from the prior art. The use of the compounds leads to very good properties of organic electronic devices, especially of organic electroluminescent devices, especially with regard to efficiency and operating voltage. The present invention therefore provides electronic devices, especially organic electroluminescent devices, comprising such compounds.

The present invention therefore provides a compound of formula (1)

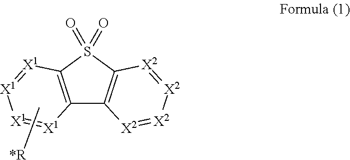

where the symbols used are as follows:
R* is a group of the following formula (2), where the dotted bond represents the bond to the base skeleton in the formula (1):

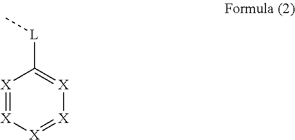

X is the same or different at each instance and is CR^{a} or N, or two adjacent X groups are a group of the following formula (3), (4) or (5), with the proviso that at least one and at most three X groups are N;

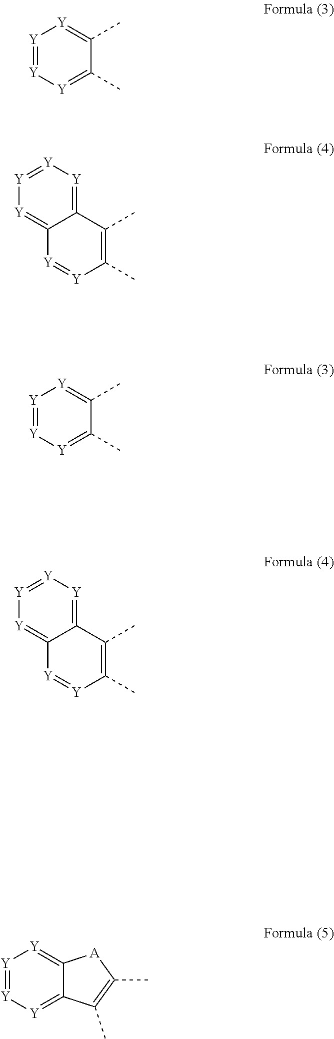

Y is the same or different at each instance and is CR^{a} or N;
A is the same or different at each instance and is NR^{a}, O, S or CR^{a}₂ X¹ is the same or different at each instance and is CR^{b} or N, with the proviso that not more than two X¹ groups are N, and with the further proviso that X¹ is C when the R* group is bonded to that X¹;
X² is the same or different at each instance and is CR^{b} or N, with the proviso that not more than two X² groups are N;
L is a single bond or an aromatic or heteroaromatic ring system which has 5 to 24 aromatic ring atoms and may be substituted by one or more R radicals;
R is the same or different at each instance and is H, D, F, Cl, Br, I, CN, NO₂, N(R¹)₂, C(═O)N(R¹)₂, C(R¹)₃, Si(R¹)₃, B(R¹)₂, C(═O)R¹, P(═O)(R¹)₂, P(R¹)₂, S(═O)R¹, S(═O)₂R¹, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C═C, Si(R¹)₂, C═O, C═S, C═Se, C═NR¹, C(═O)O, C(═O)NR¹, NR¹, P(═O)(R¹), O, S, SO or SO₂, or an aromatic ring system which has 6 to 60 aromatic ring atoms and may be substituted in each case by one or more R¹ radicals, or an electron-rich heteroaryl group which has 5 to 30 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two or more R radicals together may form an aliphatic, heteroaliphatic, aromatic or heteroaromatic ring system which may be substituted by one or more R¹ radicals;
R^{a} is the same or different at each instance and is H, D, F, Cl, Br, I, CN, NO₂, N(R¹)₂, C(═O)N(R¹)₂, C(R¹)₃, Si(R¹)₃, B(R¹)₂, C(═O)R¹, P(═O)(R¹)₂, P(R¹)₂, S(═O)R¹, S(═O)₂R¹, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C═C, Si(R¹)₂, C═O, C═S, C═Se, C═NR¹, C(═O)O, C(═O)NR¹, NR¹, P(═O)(R¹), O, S, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two or more R^{a} radicals together may form an aliphatic, heteroaliphatic, aromatic or heteroaromatic ring system which may be substituted by one or more R¹ radicals;
R^{b} is the same or different at each instance and is H, D, F, CN, N(R¹)₂, C(═O)N(R¹)₂, C(R¹)₃, Si(R¹)₃, B(R¹)₂, C(═O)R¹, P(═O)(R¹)₂, P(R¹)₂, S(═O)R¹, S(═O)₂R¹, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, or an aromatic ring system which has 6 to 16 aromatic ring atoms and may be substituted by one or more R¹ radicals, or a heteroaryl group which has 5 to 30 aromatic ring atoms, is joined to the dibenzothiophene dioxide base skeleton via a carbon atom, and may be substituted by one or more R¹ radicals, or an aryloxy or heteroaryloxy group which has 5 to 60 aromatic ring atoms and may be substituted by one or more R¹ radicals; at the same time, two or more R^{b} radicals together may form an aliphatic, heteroaliphatic, aromatic or heteroaromatic ring system which may be substituted by one or more R¹ radicals;
R¹ is the same or different at each instance and is H, D, F, Cl, Br, I, CN, NO₂, N(R²)₂, C(═O)R², P(═O)(R²)₂, P(R²)₂, B(R²)₂, C(R²)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by R²C═CR², C═C, Si(R²)₂, C═O, C═S, C═Se, C═NR², C(═O)O, C(═O)NR², NR², P(═O)(R²), O, S, SO or SO₂ and where one or more hydrogen atoms may be replaced by D, F, Cl, Br, I, CN or NO₂, or an aromatic ring system which has 6 to 60 aromatic ring atoms and may be substituted in each case by one or more R² radicals, or a heteroaryl group which has 5 to 30 aromatic ring atoms, is bonded to the R, R^{a} or R^{b} group via a carbon atom, and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, where two or more R¹ radicals together may form an aliphatic or heteroaliphatic ring system that may be substituted by one or more R² radicals;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, preferably adjacent substituents R² together may form a ring system.

The present compounds may preferably be used as active compound in electronic devices. Active compounds are generally the organic or inorganic materials introduced between anode and cathode, for example in an organic electronic device, especially in an organic electroluminescent device, for example charge injection, charge transport or charge blocker materials, but especially matrix materials. Preference is given here to organic materials.

Adjacent carbon atoms in the context of the present invention are carbon atoms bonded directly to one another. In addition, “adjacent radicals” in the definition of the radicals means that these radicals are bonded to the same carbon atom or to adjacent carbon atoms. These definitions apply correspondingly, inter alia, to the terms “adjacent groups” and “adjacent substituents”.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

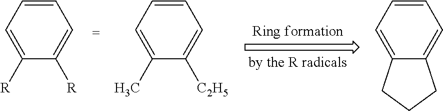

In addition, however, the abovementioned wording shall also be understood to mean that, if one of the two radicals is hydrogen, the second radical binds to the position to which the hydrogen atom was bonded, forming a ring. This will be illustrated by the following scheme:

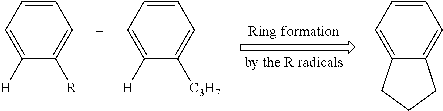

A fused aryl group, a fused aromatic ring system or a fused heteroaromatic ring system in the context of the present invention is a group in which two or more aromatic groups are fused, i.e. annelated, to one another along a common edge, such that, for example, two carbon atoms belong to the at least two aromatic or heteroaromatic rings, as, for example, in naphthalene. By contrast, for example, fluorene is not a fused aryl group in the context of the present invention, since the two aromatic groups in fluorene do not have a common edge. Corresponding definitions apply to heteroaryl groups and to fused ring systems which may but need not also contain heteroatoms.

An electron-rich heteroaryl group in the context of the invention contains 5 to 30 aromatic ring atoms, preferably 5 to 24 aromatic ring atoms, most preferably 5 to 14 aromatic ring atoms, and is a group that conducts holes, preferably selected from the group of the dibenzofurans, dibenzothiophenes, phenoxazines, phenothiazines, carbazoles, bridged carbazoles, biscarbazoles, benzocarbazoles, indenocarbazoles, indolocarbazoles, benzofurocarbazoles, benzothioenocarbazoles, dihydroacridines, dihydrophenazines, dibenzodioxines, thianthrenes, phenoxathiines.

If two or more, preferably adjacent R, R^{a}, R^{b} and/or R² radicals together form a ring system, the result may be a monocyclic or polycyclic, aliphatic, heteroaliphatic, aromatic or heteroaromatic ring system. If two R¹ radicals together form a ring system, the result may be a monocyclic or polycyclic, aliphatic or heteroaliphatic ring system.

An aryl group in the context of this invention contains 6 to 60 carbon atoms, preferably 6 to 40 carbon atoms, more preferably 6 to 30 carbon atoms; a heteroaryl group in the context of this invention contains 2 to 60 carbon atoms, preferably 2 to 40 carbon atoms, more preferably 2 to 30 carbon atoms, and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused aryl or heteroaryl group, for example naphthalene, anthracene, phenanthrene, quinoline, isoquinoline, etc.

An aromatic ring system in the context of this invention contains 6 to 60 carbon atoms, preferably 6 to 40 carbon atoms, more preferably 6 to 30 carbon atoms, in the ring system. A heteroaromatic ring system in the context of this invention contains 1 to 60 carbon atoms, preferably 1 to 40 carbon atoms, more preferably 1 to 30 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ethers, stilbene, etc. shall thus also be regarded as aromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, shall likewise be regarded as an aromatic or heteroaromatic ring system.

A cyclic alkyl, alkoxy or thioalkoxy group in the context of this invention is understood to mean a monocyclic, bicyclic or polycyclic group.

In the context of the present invention, a C1- to C20-alkyl group in which individual hydrogen atoms or CH₂ groups may also be substituted by the abovementioned groups is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals. An alkenyl group is understood to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is understood to mean, for example, ethynyl, propynyl, butynyl, pentynyl, hexynyl, heptynyl or octynyl. A C1- to C40-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system which has 5 to 60, preferably 5-40, aromatic ring atoms, more preferably 5 to 30 aromatic ring atoms, and may also be substituted in each case by the abovementioned radicals and which may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

Preferred compounds in the context of the invention are compounds of the formula (1) in which a total of not more than two of the symbols X¹ and X² are N.

More preferably, not more than one of the symbols X¹ and X² is N; most preferred are compounds of the formula (6) in which all symbols X¹ and X² are CR^{b}:

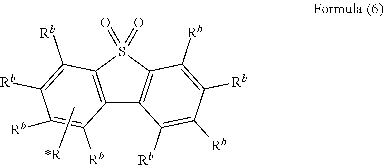

where the symbols used have the definitions given above, with the proviso that R^{b} is absent when the R* group is bonded to that position.

A preferred embodiment of the invention is compounds of the formula (6-1) to (6-4). Particular preference is given to compounds of the formulae (6-1), (6-2) and (6-3). Very particular preference is given to compounds of the formula (6-1) and of the formula (6-3).

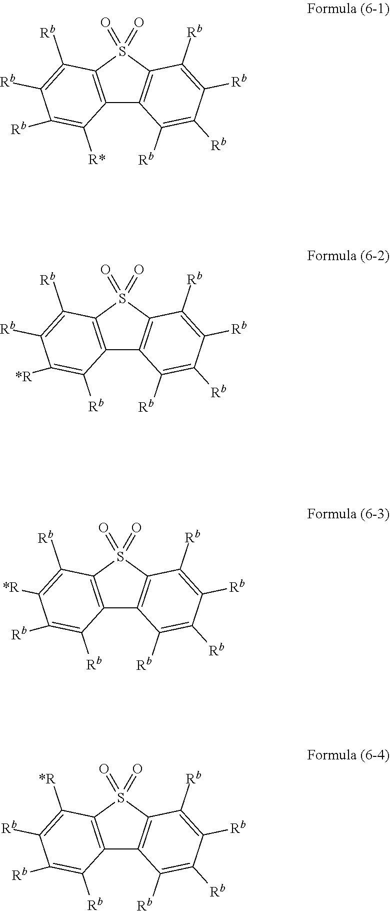

In a further preferred embodiment of the invention, not more than two R^{b} radicals are a group other than H and D. More preferably, not more than one R^{b} radical or none of the R^{b} radicals is a group other than H and D. The compounds are preferably selected from compounds of the formulae (6-1a) to (6-4f), more preferably from the compounds of the formulae (6-1a) to (6-3e) and most preferably from the compounds of the formulae (6-1a) to (6-1e) and of the formulae (6-3a) to (6-3e):

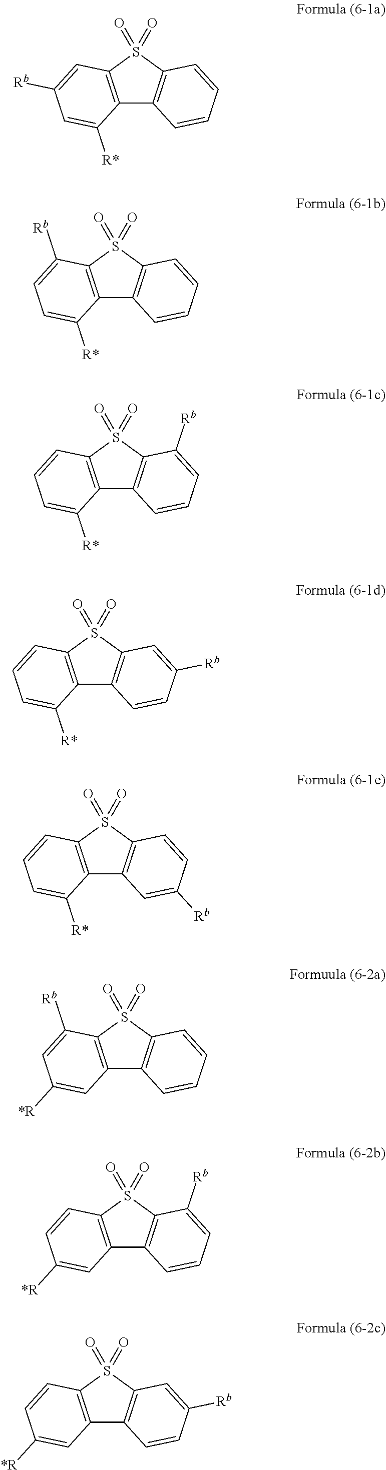

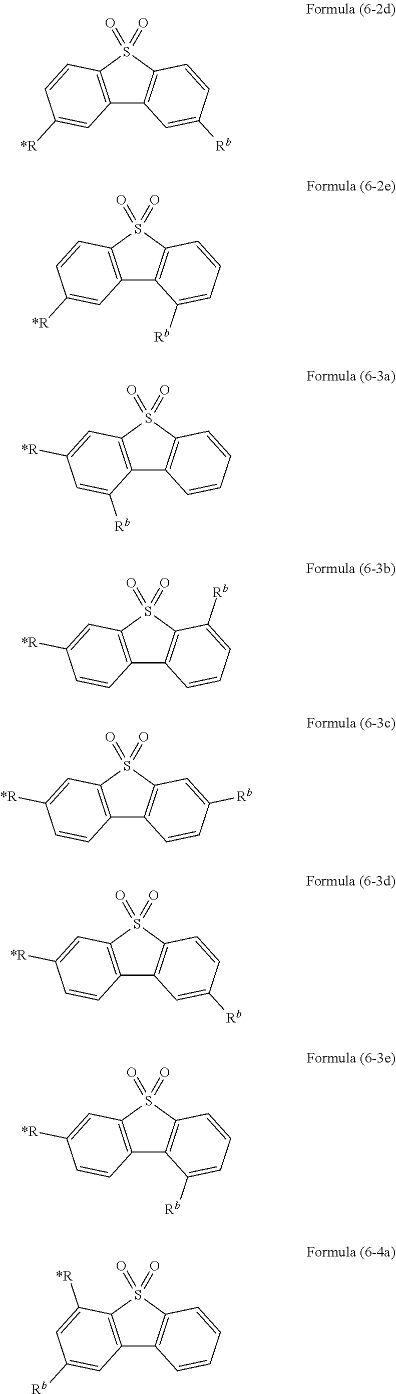

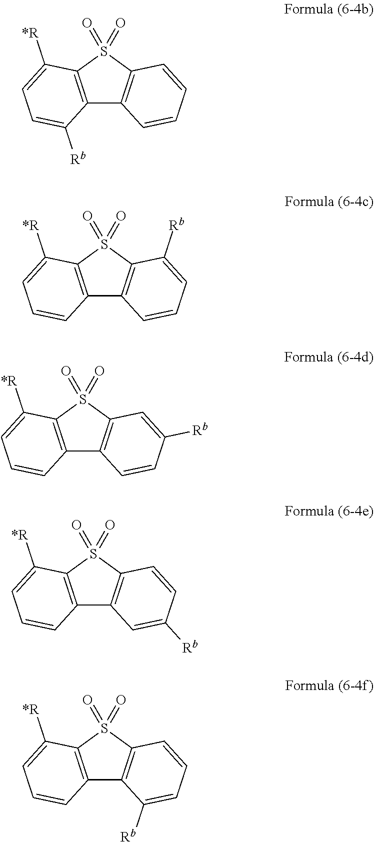

where the symbols used have the definitions given above.

In a preferred embodiment of the formula (2), all X are the same or different and are CR or N, with the proviso that at least one X and at most three X are N. These are preferably structures of the following formula (7):

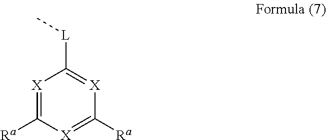

where the symbols used have the definitions given above, 1, 2 or 3 X are N, and R^{a} is preferably the same or different at each instance and is an aromatic or heteroaromatic ring system which has 5 to 40 aromatic ring atoms and may be substituted by one or more R¹ radicals.

Preferred embodiments of the formula (7) are the groups of the following formulae (7a), (7b) and (7c), particular preference being given to the groups of the formula (7a),

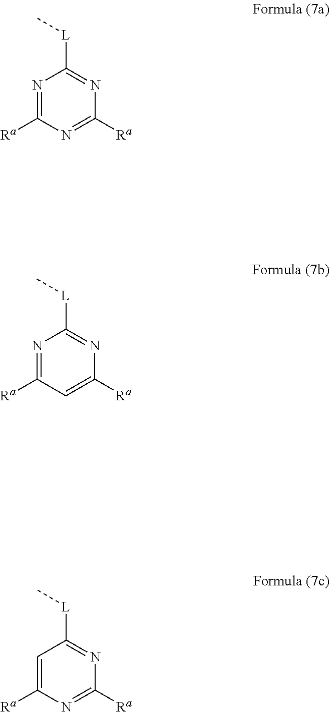

where the symbols used have the definitions given above and R^{a} is preferably the same or different at each instance and is an aromatic or heteroaromatic ring system which has 5 to 40 aromatic ring atoms and may be substituted by one or more R¹ radicals.

In a further preferred embodiment of the formula (2), two adjacent X are a group of the formula (3) or (4), where Y is the same or different and is CR^{a}, and, of the remaining X, exactly two X are N and the third X is CR^{a}, such that the structure is one according to one of the following formulae (8) to (13):

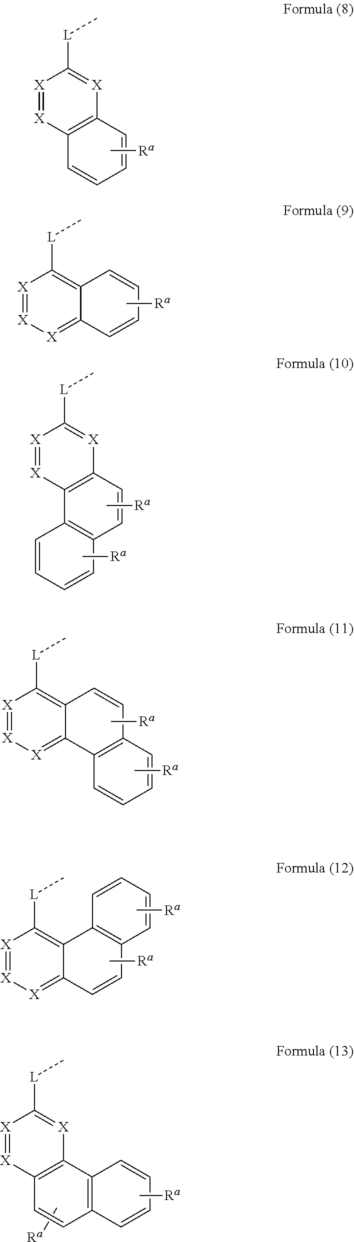

where the symbols have the definitions given above and exactly two X groups are N.

In a further preferred embodiment of the formula (2), two adjacent X are a group of the formula (4), where exactly one Y group is N and the remaining Y are CR^{a}, and exactly one X group is N and the remaining X are CR^{a}, such that the structure is one of the formulae (14) to (17):

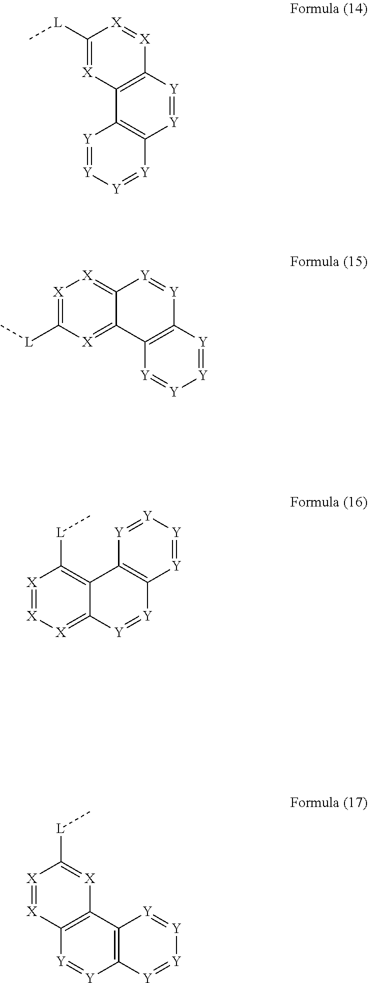

where the symbols have the definitions given above and exactly one X group and exactly one Y group are N.

In a further preferred embodiment of the formula (2), two adjacent X are a group of the formula (5), where Y is the same or different and is CR^{a}, and, of the remaining X, exactly two X are N and the third X is CR^{a}, such that the structure is one according to one of the following formulae (18) to (21):

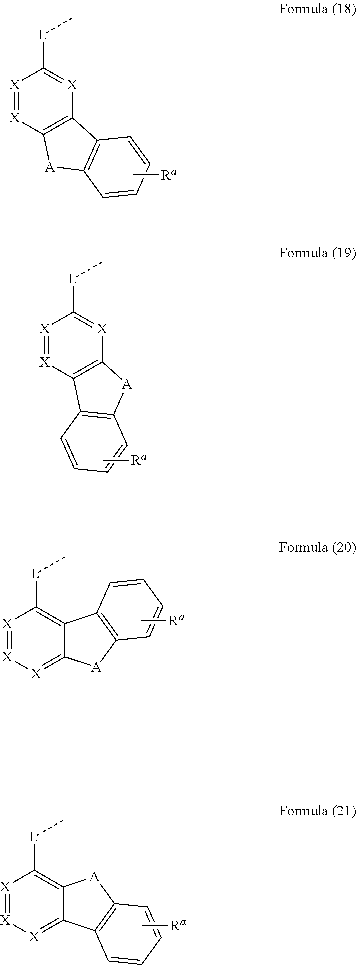

where the symbols have the definitions given above and exactly two X groups are N.

Preferred embodiments of the formulae (8) to (21) are the structures of the following formulae (8a) to (21a):

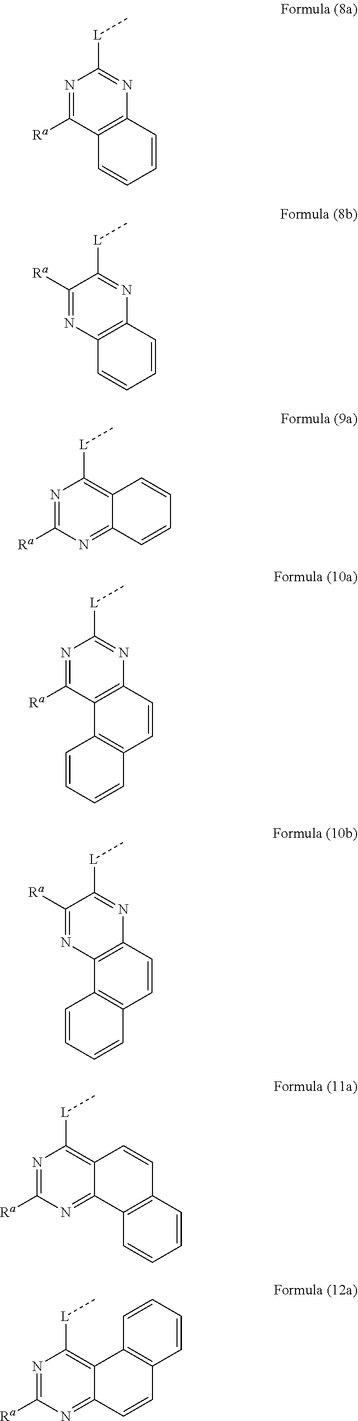

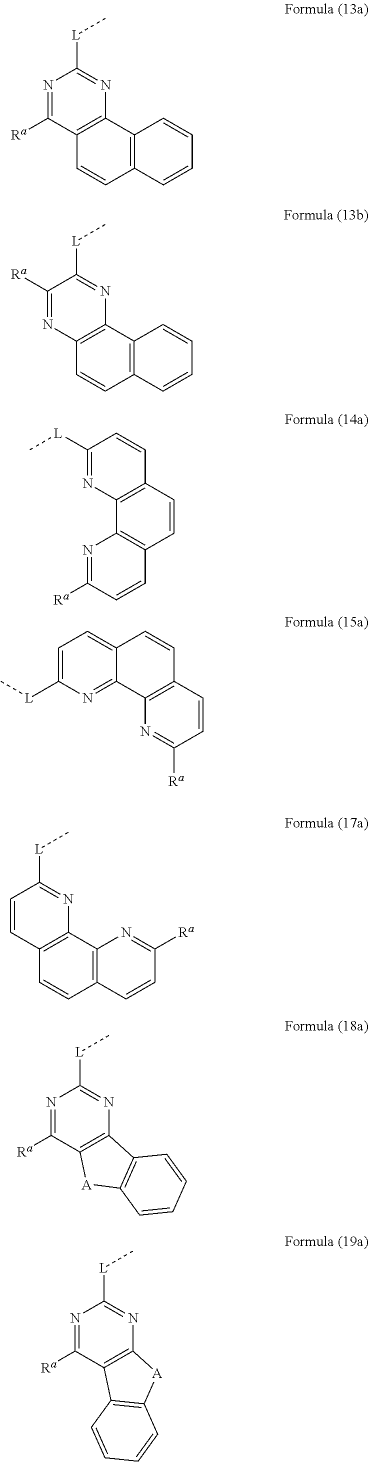

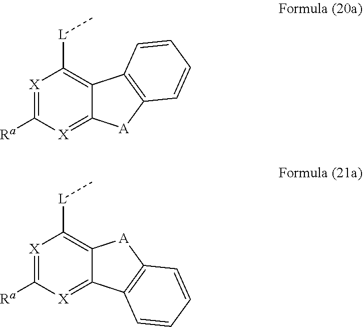

where the symbols used have the definitions given above and R^{a} is preferably the same or different at each instance and is H, D or an aromatic or heteroaromatic ring system which has 5 to 40 aromatic ring atoms and may be substituted by one or more R¹ radicals.

In a preferred embodiment of the formulae (8) to (21) or (8a) to (21a), R^{a} is the same or different at each instance and is H, D or an aromatic or heteroaromatic ring system which has 6 to 30 aromatic ring atoms and may be substituted by one or more R¹ radicals. More preferably, R^{a} is the same or different at each instance and is H, D or an aromatic or heteroaromatic ring system which has 6 to 24 aromatic ring atoms, especially 6 to 13 aromatic ring atoms, and may be substituted by one or more preferably nonaromatic R¹ radicals. In the formulae (8a) to (21a), R^{a} is most preferably selected from H, D, phenyl, d₅-phenyl, meta- or para-biphenyl, dibenzofuran or carbazole, where these groups may each be substituted by one or more R¹ radicals, but are preferably unsubstituted.

In a particularly preferred embodiment of the formulae (18) to (21) and (18a) to (21a), A is NR^{a}, O or S, especially O or NR^{a}.

In a preferred embodiment of the invention, the L group is a single bond or a divalent aromatic or heteroaromatic ring system which has 6 to 18 aromatic ring atoms and may be substituted in each case by one or more R radicals. More preferably, L is a single bond or an aromatic ring system which has 6 to 12 aromatic ring atoms and may be substituted by one or more R radicals, or a dibenzofuran or dibenzothiophene group that may be substituted by one or more R radicals. Most preferably, L is a single bond, a meta- or para-bonded phenylene group that may be substituted by one or more R radicals, or a dibenzofuran or dibenzothiophene group that may be substituted in each case by one or more R radicals. The dibenzofuran or dibenzothiophene group is preferably bonded in the 1,3, 1,6, 1,7, 1,8, 3,6, 3,8 or 3,9 position. The preference that L may be a dibenzofuran or dibenzothiophene group applies especially when the heteroaryl group of the R* radical is a group of the formula (7). Especially preferably, L is a single bond or a meta- or para-bonded phenylene group or a dibenzofuran group, each of which may be substituted by one or more R radicals, where the R group is preferably H or D; most preferably, the R group is H.

When L is an aromatic or heteroaromatic ring system, this is preferably selected from the structures of the following formulae (L-1) to (L-34):

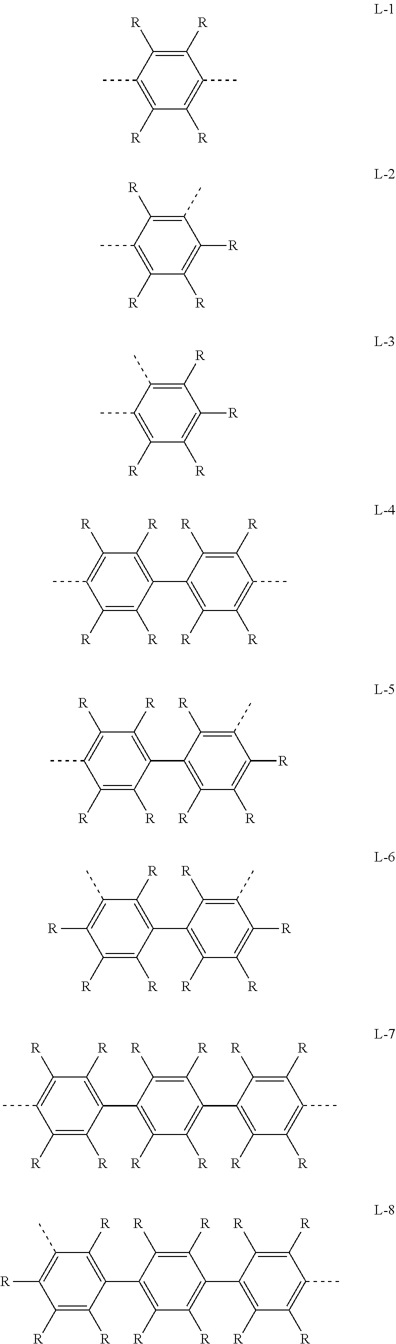

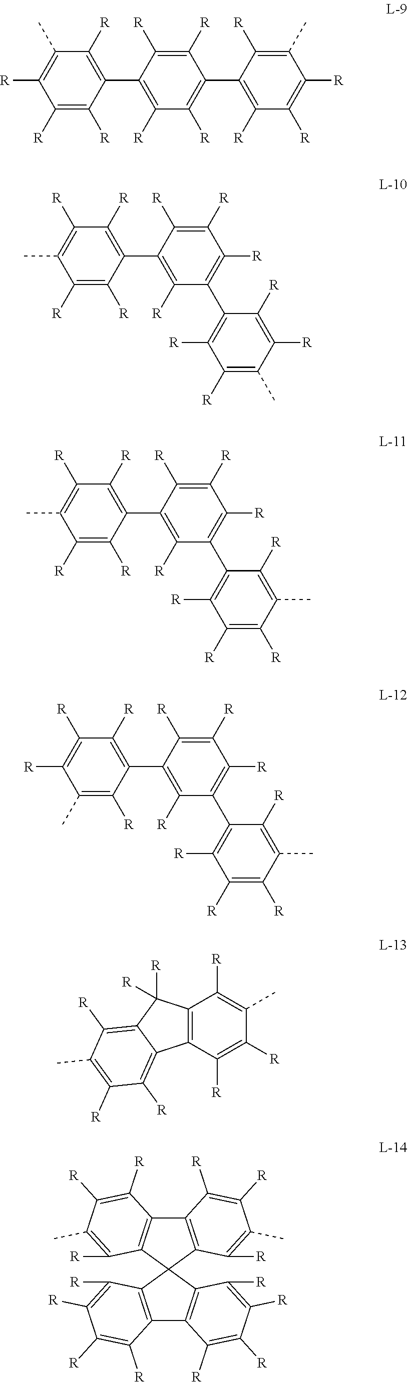

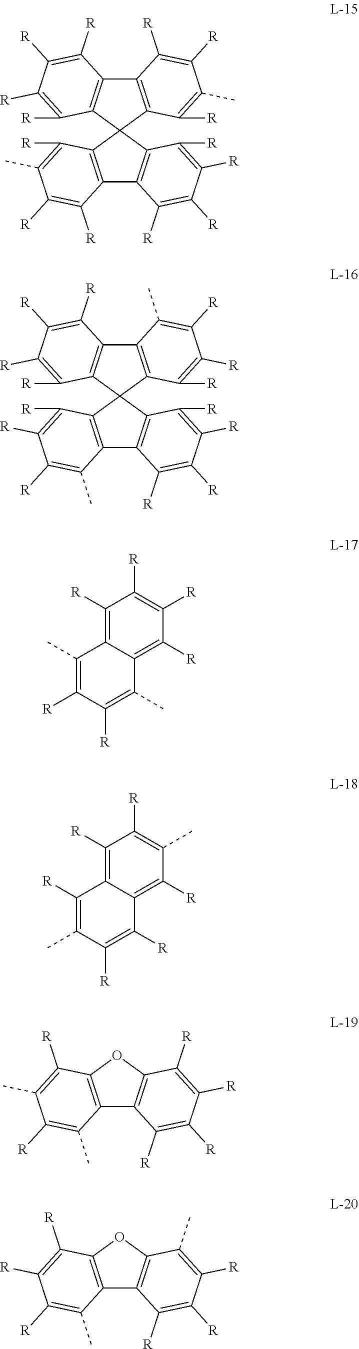

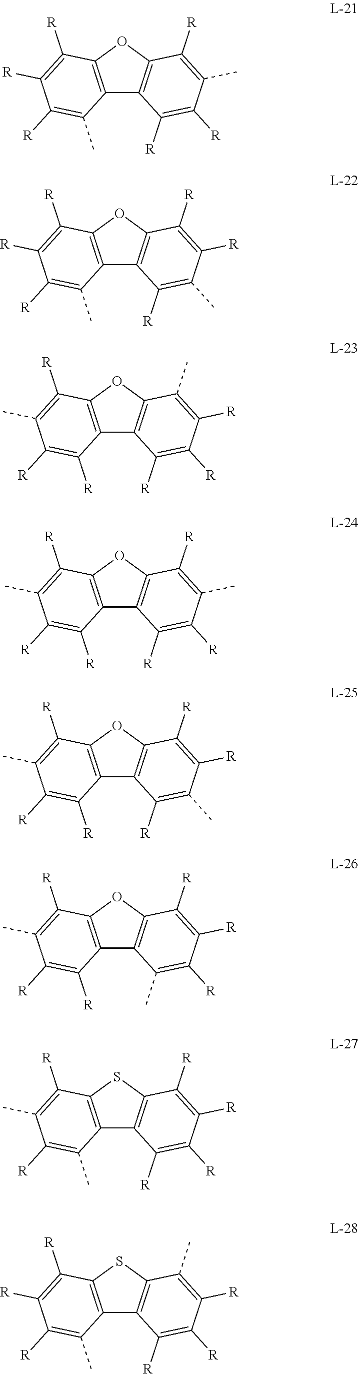

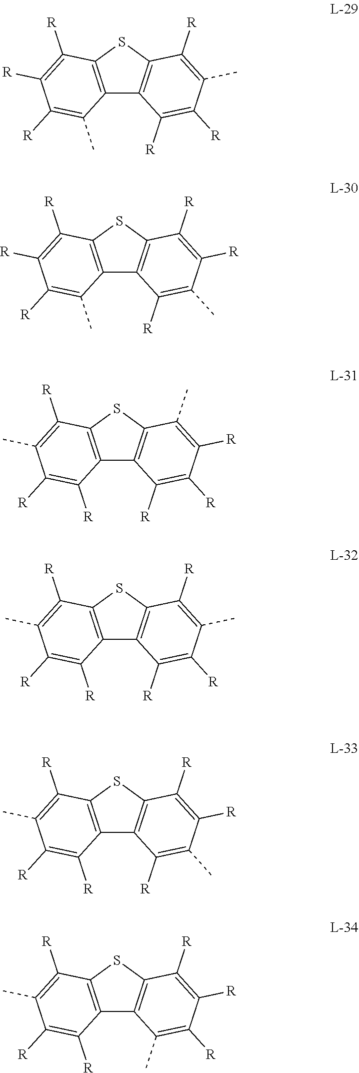

where the symbols used have the meanings given above and the dotted bonds represent the bonds to the heteroaryl group in formula (2) and to the base skeleton of the compound of the formula (1).

More preferably, L is a single bond, an optionally substituted phenylene or dibenzofuran group, i.e. a group of the formula (L-1) to (L-3) or (L19) to (L26), especially (L-1), (L-2) or (L-19) to (L26).

There follows a description of preferred substituents R, R^{a}, R^{b}, R¹ and R² in the compounds of the invention. In a particularly preferred embodiment of the invention, the preferences specified hereinafter for R, R^{a}, R^{b}, R¹ and R² occur simultaneously and are applicable to the structures of the formula (1) and to all preferred embodiments.

In a preferred embodiment of the invention, R is the same or different at each instance and is selected from the group consisting of H, D, F, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic ring system which has 6 to 30 aromatic ring atoms and may be substituted in each case by one or more R¹ radicals, or an electron-rich heteroaryl group which has 5 to 30 aromatic ring atoms and may be substituted in each case by one or more R¹ radicals; at the same time, two R radicals together may also form an aliphatic, aromatic or heteroaromatic ring system. More preferably, R is the same or different at each instance and is selected from the group consisting of H, D, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted in each case by one or more R¹ radicals, but is preferably unsubstituted, or an aromatic ring system which has 6 to 24 aromatic ring atoms and may be substituted in each case by one or more R¹ radicals, preferably nonaromatic R¹ radicals, or an electron-rich heteroaryl group which has 5 to 24 aromatic ring atoms and may be substituted in each case by one or more R¹ radicals, preferably nonaromatic R¹ radicals. Most preferably, R is the same or different at each instance and is selected from the group consisting of H, D or an aromatic ring system which has 6 to 14 aromatic ring atoms and may be substituted in each case by one or more R¹ radicals, preferably nonaromatic R¹ radicals, or an electron-rich heteroaryl group which has 5 to 24 aromatic ring atoms and may be substituted in each case by one or more R¹ radicals, preferably nonaromatic R¹ radicals. Especially preferably, R is the same or different at each instance and is selected from the group consisting of H, D or an aromatic ring system which has 6 to 14 aromatic ring atoms and may be substituted in each case by one or more R¹ radicals, preferably nonaromatic R¹ radicals, or an electron-rich heteroaryl group which has 5 to 14 aromatic ring atoms and may be substituted in each case by one or more R¹ radicals, preferably nonaromatic R¹ radicals.

In a preferred embodiment of the invention, R^{a} is the same or different at each instance and is selected from the group consisting of H, D, F, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R^{a} radicals together may also form an aliphatic, aromatic or heteroaromatic ring system. More preferably, R^{a} is the same or different at each instance and is selected from the group consisting of H, D, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R^{a} is the same or different at each instance and is selected from the group consisting of H, D or an aromatic or heteroaromatic ring system which has 6 to 24 aromatic ring atoms and may be substituted in each case by one or more R¹ radicals, preferably nonaromatic R¹ radicals. Especially preferably, R^{a} is the same or different at each instance and is selected from the group consisting of H, D or an aromatic or heteroaromatic ring system which has 6 to 14 aromatic ring atoms and may be substituted in each case by one or more R¹ radicals, preferably nonaromatic R¹ radicals.

The substituents R^{b} on the dibenzothiophene dioxide base skeleton are preferably the same or different at each instance and are selected from a group consisting of H, D, F, CN, OR¹, NR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic ring system which has 6 to 16 aromatic ring atoms and may be substituted in each case by one or more R¹ radicals, or a heteroaryl group which has 5 to 30 aromatic ring atoms, is bonded to the dibenzothiophene dioxide base skeleton via a carbon atom and may be substituted in each case by one or more R¹ radicals. More preferably, R^{b} is the same or different at each instance and is selected from the group consisting of H, D, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted in each case by one or more R¹ radicals, but is preferably unsubstituted, or an aromatic ring system which has 6 to 16 aromatic ring atoms and may be substituted in each case by one or more R¹ radicals, preferably nonaromatic R¹ radicals, or a heteroaryl group which has 5 to 24 aromatic ring atoms and may be substituted in each case by one or more R¹ radicals, preferably nonaromatic R¹ radicals, where the heteroaromatic ring system is bonded to the dibenzothiophene dioxide base skeleton via a carbon atom. Most preferably, R^{b} is the same or different at each instance and is selected from the group consisting of H, D or an aromatic ring system which has 6 to 16 aromatic ring atoms and may be substituted in each case by one or more R¹ radicals, preferably nonaromatic R¹ radicals, or a heteroaryl group which has 5 to 24 aromatic ring atoms and may be substituted in each case by one or more R¹ radicals, preferably nonaromatic R¹ radicals, where the heteroaryl group is bonded to the dibenzothiophene dioxide base skeleton via a carbon atom. Especially preferably, the nonaromatic R¹ radicals are H or D.

Suitable aromatic or heteroaromatic ring systems R are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position or via the nitrogen atom, dibenzofuran which may be joined via the 1, 2, 3 or 4 position, dibenzothiophene which may be joined via the 1, 2, 3 or 4 position, indenocarbazole, indolocarbazole, benzimidazole, phenanthrene, triphenylene or a combination of two or three of these groups, each of which may be substituted by one or more R¹ radicals.

Suitable aromatic or heteroaromatic ring systems R^{a} are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position or via the nitrogen atom, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R^{a} is a heteroaryl group, especially triazine, pyrimidine, quinazoline or carbazole, preference may also be given to aromatic or heteroaromatic R¹ radicals on this heteroaryl group.

Suitable aromatic or heteroaromatic ring systems R^{b} are selected from phenyl, biphenyl, especially ortho-, meta- or para-biphenyl, fluorene which may be joined via the 1, 2, 3 or 4 position, naphthalene which may be joined via the 1 or 2 position, indole, benzofuran, benzothiophene, carbazole which may be joined to the dibenzothiophene dioxide base skeleton via a carbon atom, dibenzofuran which may be joined via the 1, 2, 3 or 4 position, dibenzothiophene which may be joined via the 1, 2, 3 or 4 position, indenocarbazole, indolocarbazole which may be joined to the dibenzothiophene dioxide base skeleton via a carbon atom, pyridine, pyrimidine, pyrazine, pyridazine, triazine, quinoline, quinazoline, benzimidazole, phenanthrene or a combination of two or three of these groups, each of which may be substituted by one or more R¹ radicals. When R^{b} is a heteroaryl group, especially triazine, pyrimidine, quinazoline or carbazole, preference may also be given to aromatic or heteroaromatic R¹ radicals on this heteroaryl group.

The R^{a} groups here in the formulae (7) to (21a), when they are an aromatic or heteroaromatic ring system, are preferably selected from the groups of the formulae R1 to R83. The R groups, when they are an aromatic or heteroaromatic ring system, are preferably selected from the groups of the formulae R-1 to R-46 and R-67 to R-75. The R^{b} groups, when they are an aromatic or heteroaromatic ring system, are preferably selected from the groups of the formulae R-1 to R-4, R12 to R-42, R-47 to R74 and R-76 to R-83.

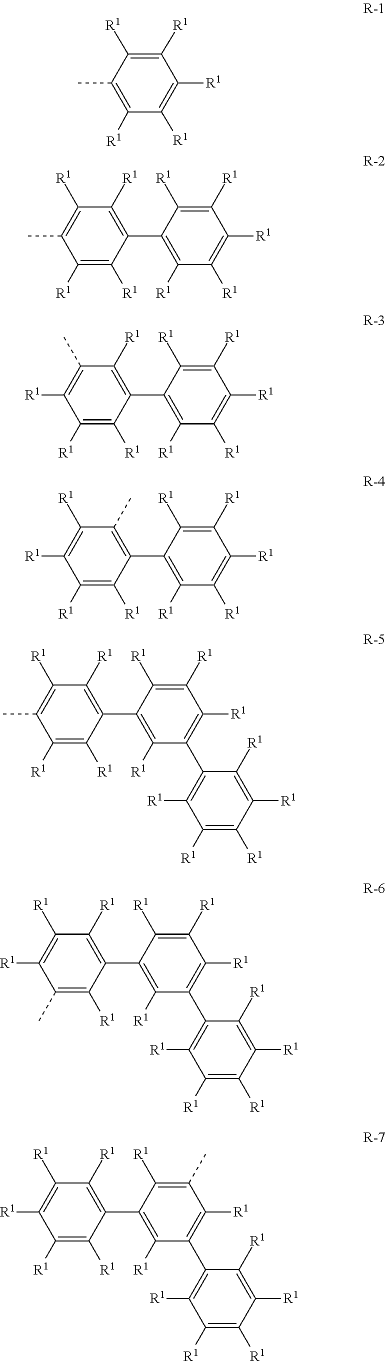

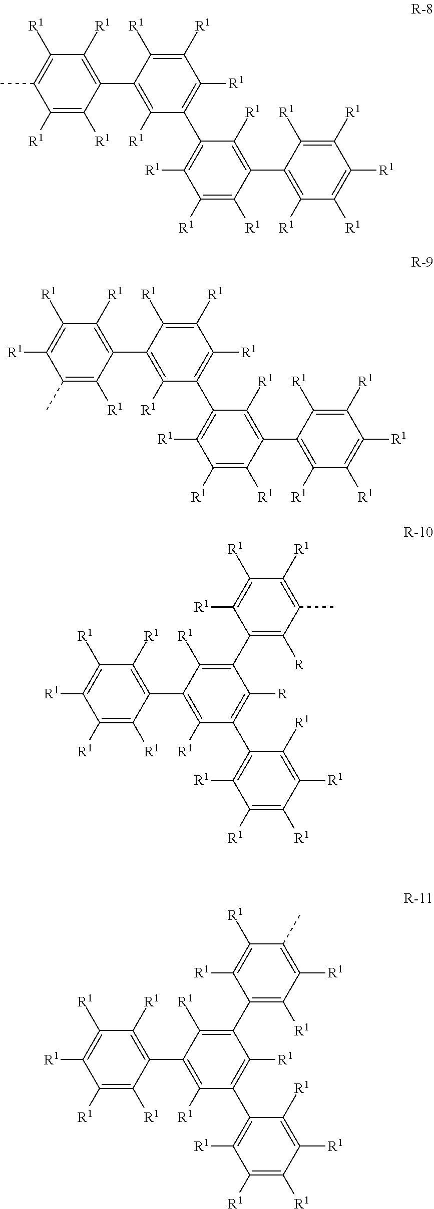

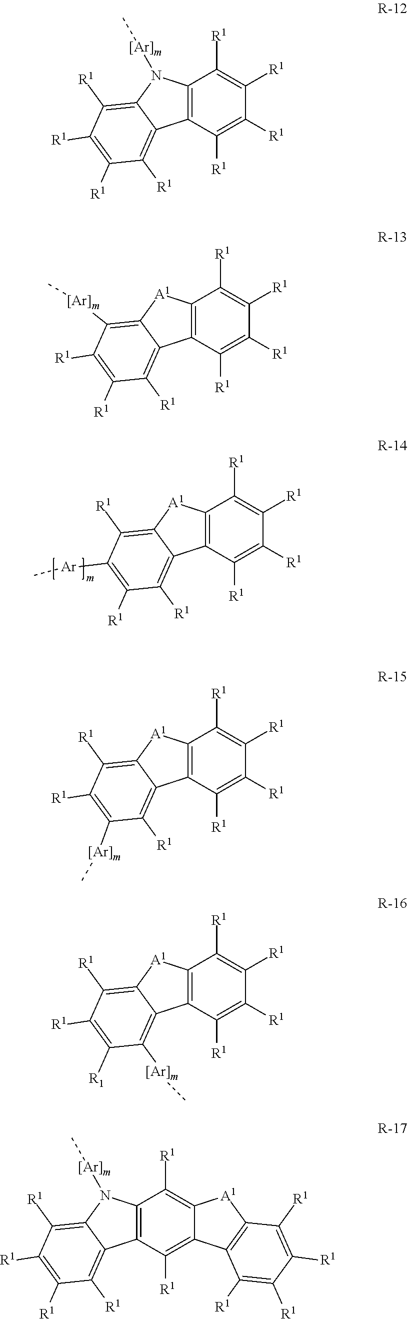

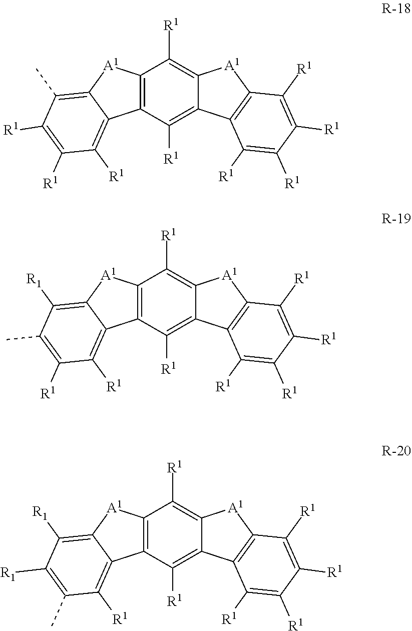

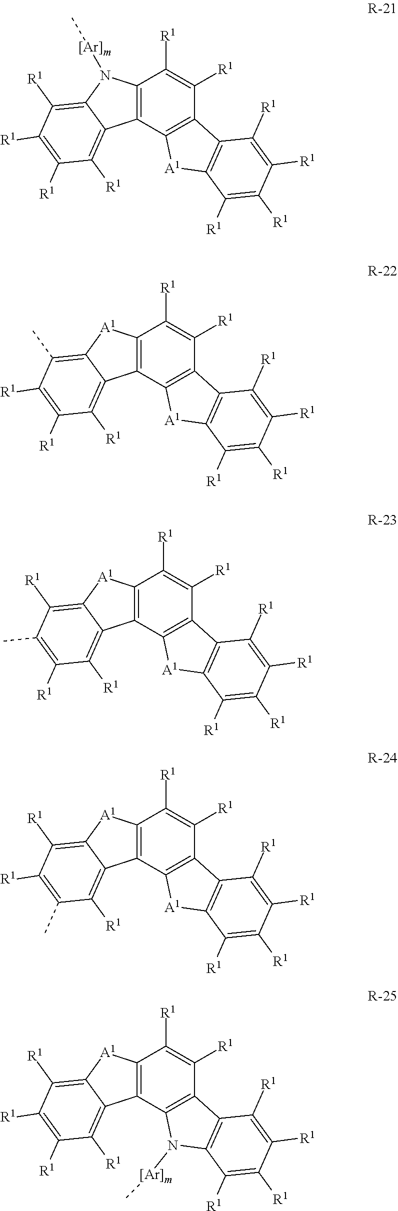

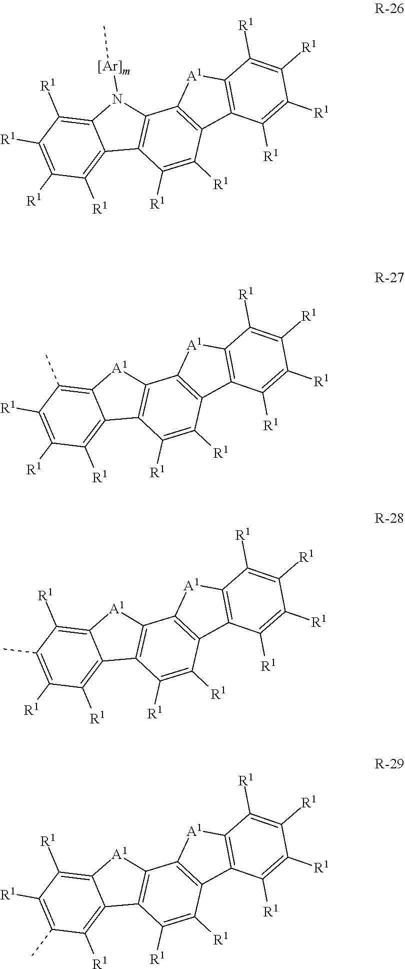

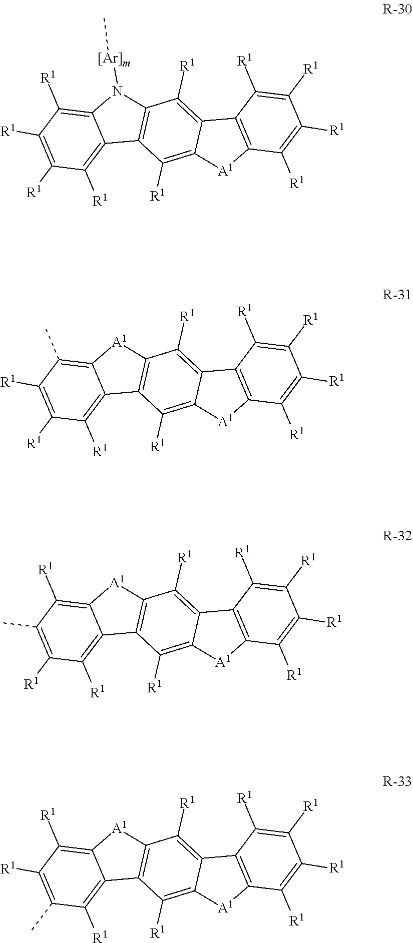

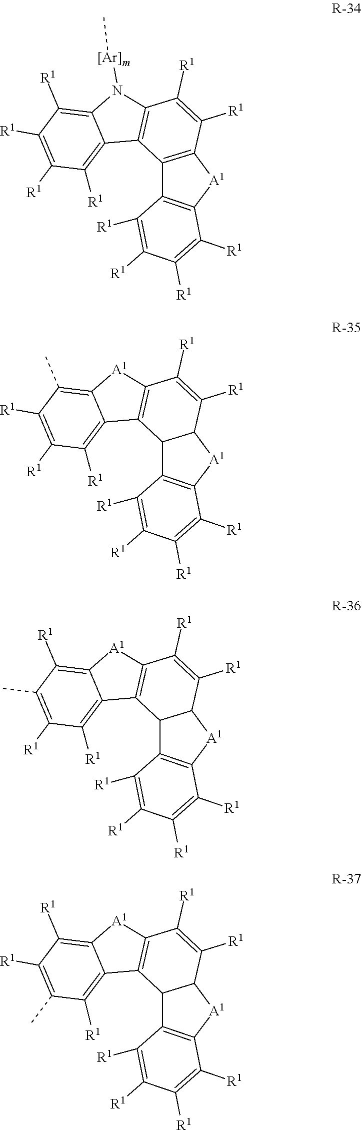

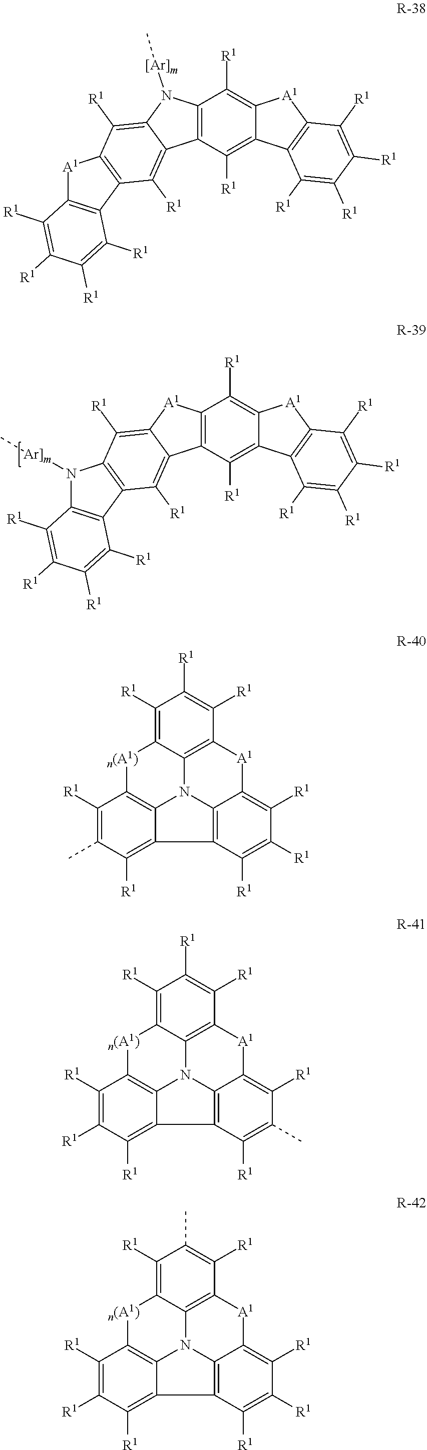

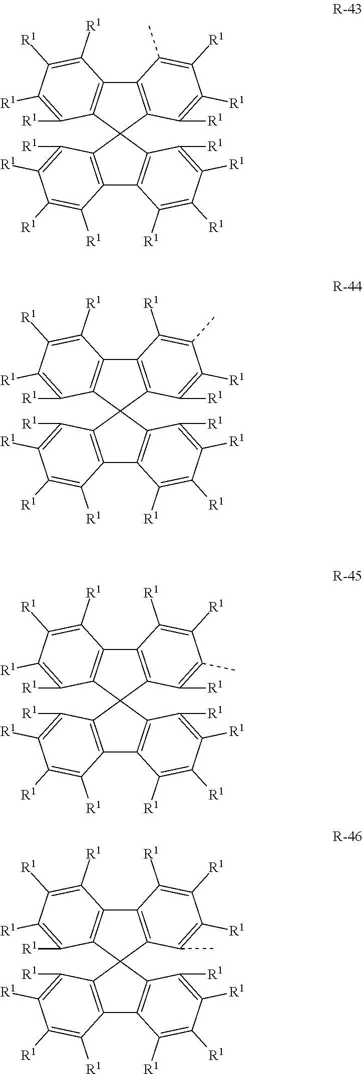

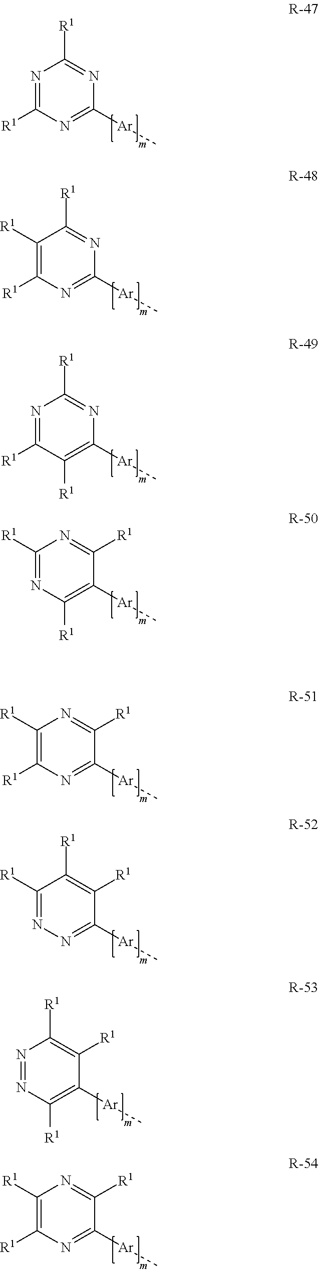

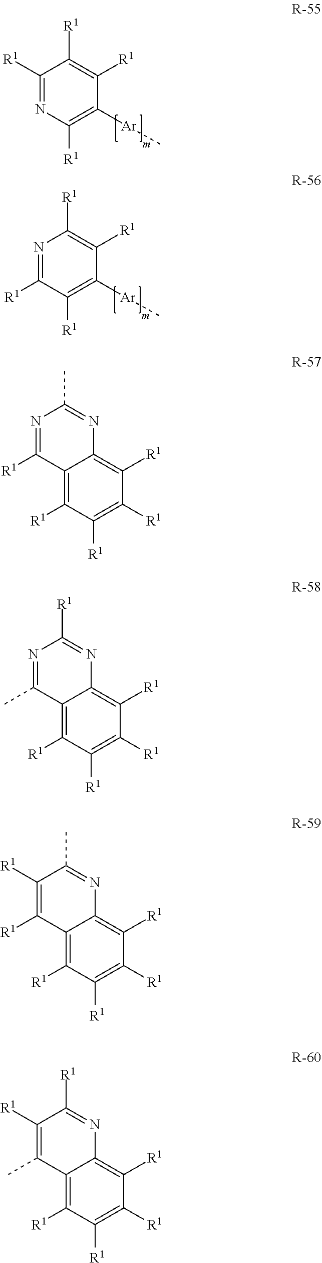

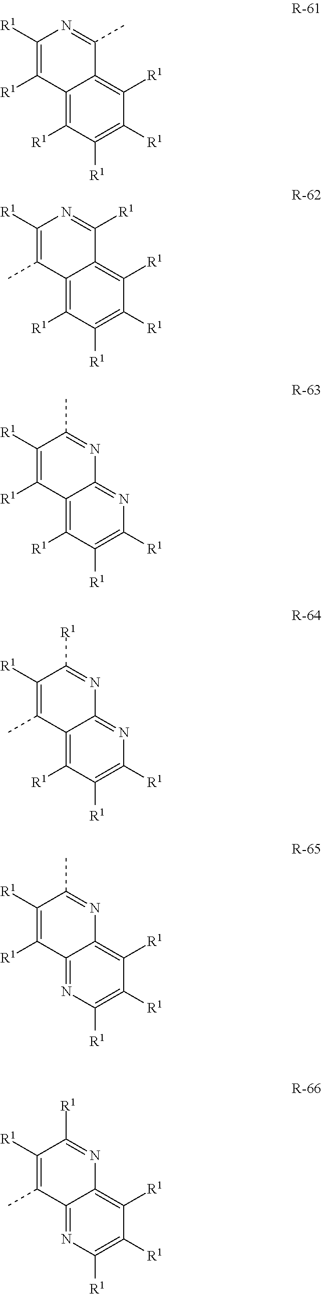

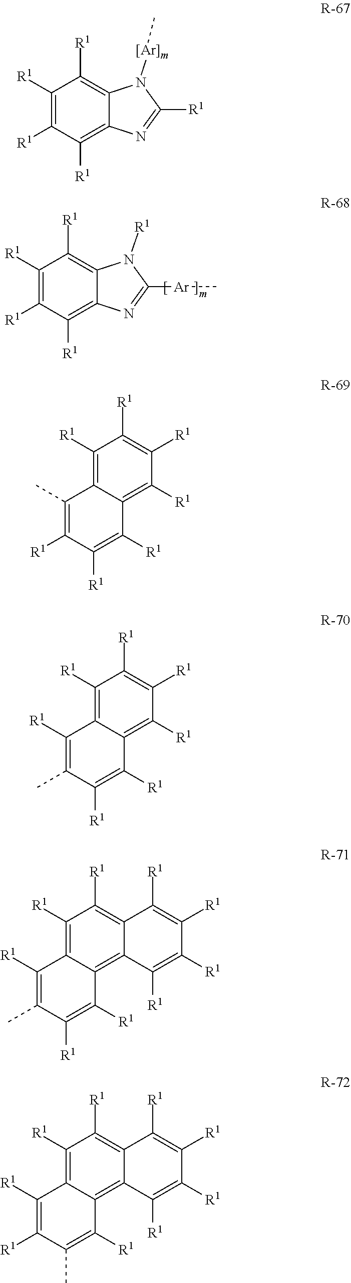

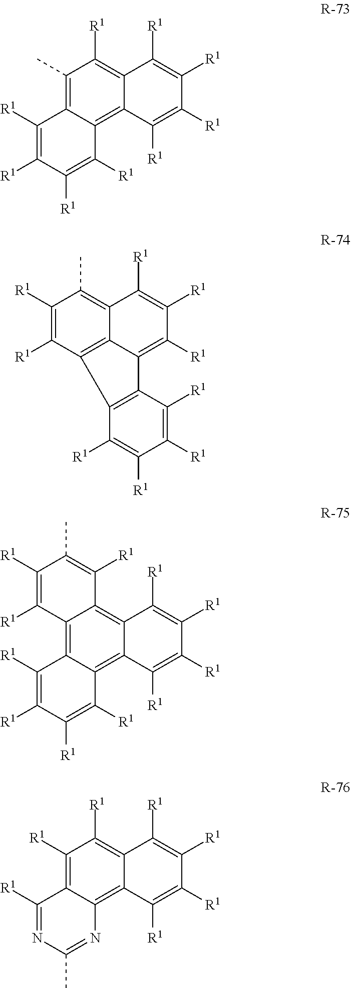

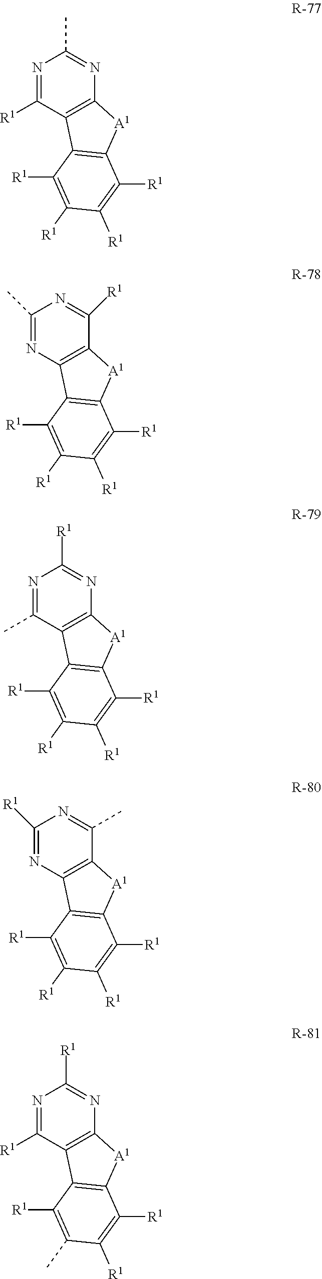

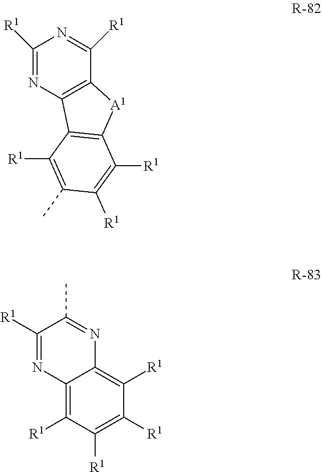

where R¹ has the definitions given above, the dotted bond represents the position of the bond of the group and in addition:
Ar is the same or different at each instance and is a bivalent aromatic or heteroaromatic ring system which has 6 to 18 aromatic ring atoms and may be substituted in each case by one or more R¹ radicals;
A¹ is the same or different at each instance and is C(R¹)₂, NR¹, O or S; with the proviso that, in the R1-R83 groups that have two or more A¹ groups, at least one A¹ group is NR¹ or O.
n is 0 or 1, where n=0 means that no A¹ group is bonded at this position and R¹ radicals are bonded to the corresponding carbon atoms instead;
m is 0 or 1, where m=0 means that the Ar group is absent is bonded;

When the abovementioned R-1 to R-83 groups have two or more A¹ groups, possible options for these include all combinations from the definition of A¹. Preferred embodiments in that case are those in which one A¹ group is NR¹ and the other A¹ group is C(R¹)₂ or in which both A¹ groups are NR¹ or in which both A¹ groups are O. In a particularly preferred embodiment of the invention, in R-1 to R-83 groups having two or more A¹ groups, at least one A¹ group is O or is NR¹.

When A¹ is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl having bonding patterns as listed above for R-1 to R-11, where these structures may be substituted by one or more R² radicals, but are preferably unsubstituted.

When A¹ is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R² radicals together may form an aliphatic, heteroaliphatic, aromatic or heteroaromatic ring system. In a particularly preferred embodiment of the invention, R¹ is the same or different at each instance and is selected from the group consisting of H, D, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms, preferably 6 to 13 aromatic ring atoms, and may be substituted in each case by one or more R² radicals, but is preferably unsubstituted.

When R¹ is an aromatic or heteroaromatic ring system, it is preferably selected from the above-depicted structures (R-1) to (R-83), in which case these structures are substituted by R² rather than R¹.

In a further preferred embodiment of the invention, R² is the same or different at each instance and is H, D, F, an alkyl group having 1 to 4 carbon atoms or an aryl group having 6 to 10 carbon atoms, which may be substituted by an alkyl group having 1 to 4 carbon atoms, but is preferably unsubstituted.

At the same time, the alkyl groups in compounds of the invention which are processed by vacuum evaporation preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable that the R, R^{a}, R^{b}, R¹ and R² groups do not contain any fused aryl or heteroaryl groups in which two or more six-membered rings are fused directly to one another. An exception to this is formed by phenanthrene, triphenylene, quinazoline and quinoxaline, which, because of their higher triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of suitable compounds according to the above-detailed embodiments are the compounds detailed in the following table:

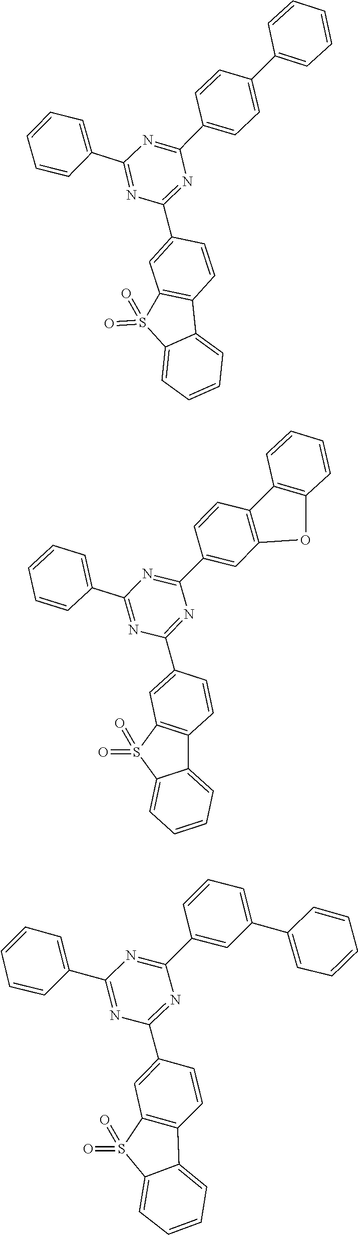 [SMILES: O=S1(=O)c2ccccc2-c2ccc(-c3nc(-c4ccccc4)nc(-c4ccc(-c5ccccc5)cc4)n3)cc21 ; O=S1(=O)c2ccccc2-c2ccc(-c3nc(-c4ccccc4)nc(-c4ccc5c(c4)oc4ccccc45)n3)cc21 ; O=S1(=O)c2ccccc2-c2ccc(-c3nc(-c4ccccc4)nc(-c4cccc(-c5ccccc5)c4)n3)cc21]

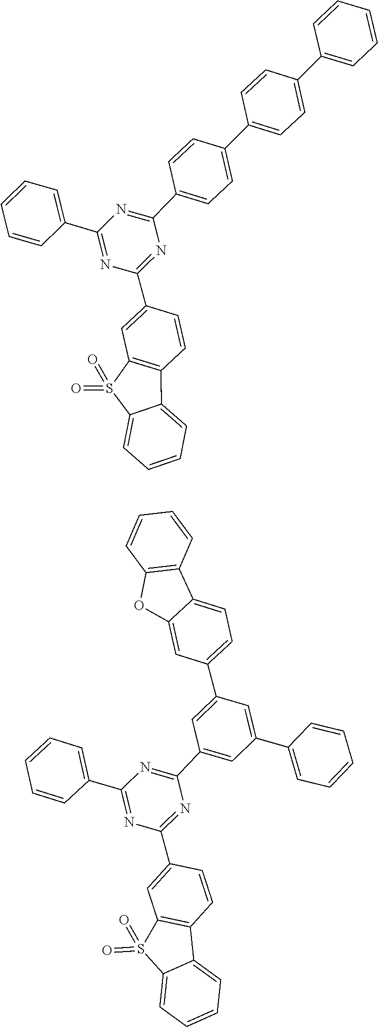 [SMILES: O=S1(=O)c2ccccc2-c2ccc(-c3nc(-c4ccccc4)nc(-c4ccc(-c5ccc(-c6ccccc6)cc5)cc4)n3)cc21 ; O=S1(=O)c2ccccc2-c2ccc(-c3nc(-c4ccccc4)nc(-c4cc(-c5ccccc5)cc(-c5ccc6c(c5)oc5ccccc56)c4)n3)cc21]

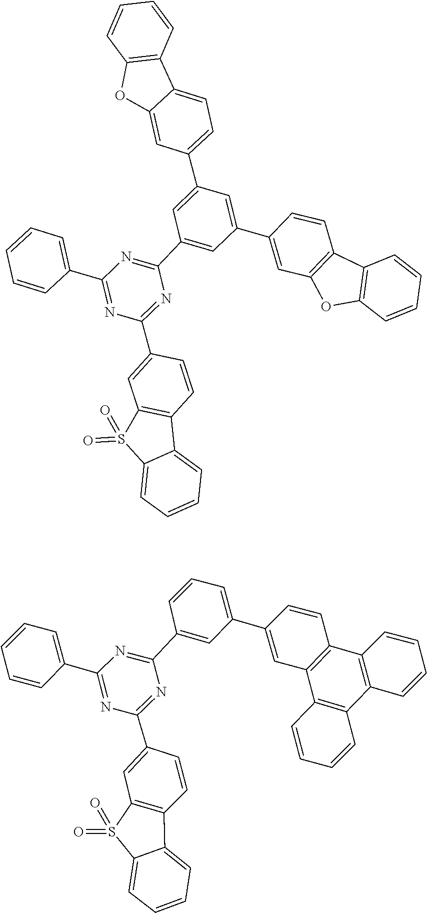 [SMILES: O=S1(=O)c2ccccc2-c2ccc(-c3nc(-c4ccccc4)nc(-c4cc(-c5ccc6c(c5)oc5ccccc56)cc(-c5ccc6c(c5)oc5ccccc56)c4)n3)cc21 ; O=S1(=O)c2ccccc2-c2ccc(-c3nc(-c4ccccc4)nc(-c4cccc(-c5ccc6c7ccccc7c7ccccc7c6c5)c4)n3)cc21]

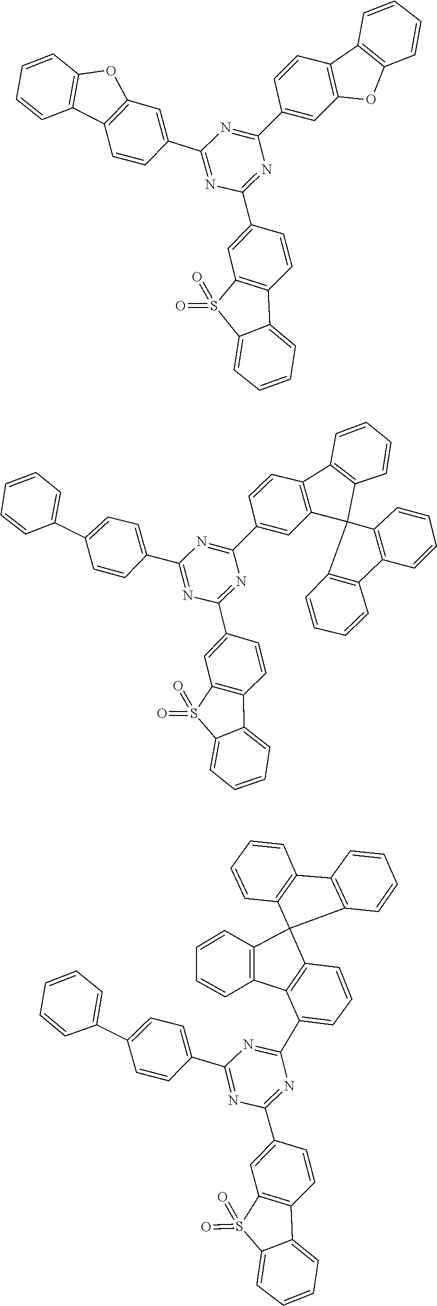 [SMILES: O=S1(=O)c2ccccc2-c2ccc(-c3nc(-c4ccc5c(c4)oc4ccccc45)nc(-c4ccc5c(c4)oc4ccccc45)n3)cc21 ; O=S1(=O)c2ccccc2-c2ccc(-c3nc(-c4ccc(-c5ccccc5)cc4)nc(-c4ccc5c(c4)C4(c6ccccc6-c6ccccc64)c4ccccc4-5)n3)cc21 ; O=S1(=O)c2ccccc2-c2ccc(-c3nc(-c4ccc(-c5ccccc5)cc4)nc(-c4cccc5c4-c4ccccc4C54c5ccccc5-c5ccccc54)n3)cc21]

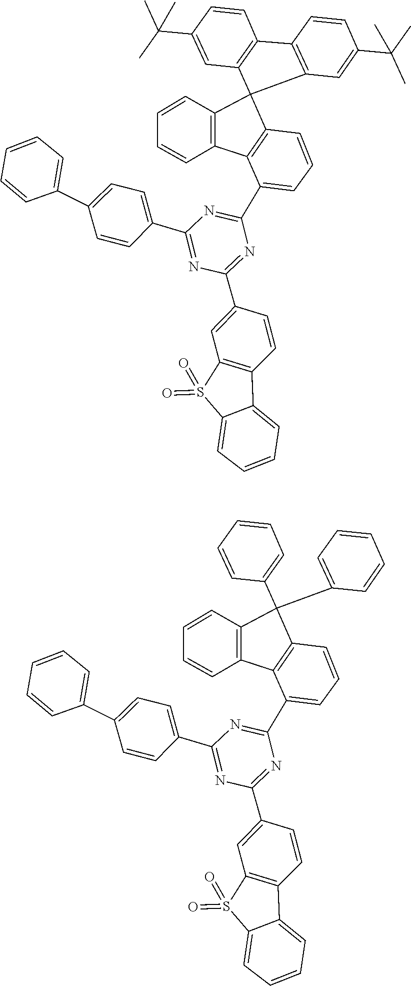 [SMILES: CC(C)(C)c1ccc2c(c1)C1(c3cc(C(C)(C)C)ccc3-2)c2ccccc2-c2c(-c3nc(-c4ccc(-c5ccccc5)cc4)nc(-c4ccc5c(c4)S(=O)(=O)c4ccccc4-5)n3)cccc21 ; O=S1(=O)c2ccccc2-c2ccc(-c3nc(-c4ccc(-c5ccccc5)cc4)nc(-c4cccc5c4-c4ccccc4C5(c4ccccc4)c4ccccc4)n3)cc21]

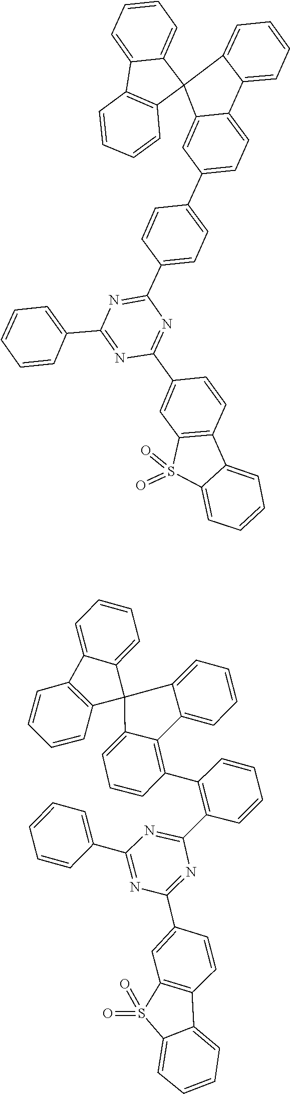 [SMILES: O=S1(=O)c2ccccc2-c2ccc(-c3nc(-c4ccccc4)nc(-c4ccc(-c5ccc6c(c5)C5(c7ccccc7-c7ccccc75)c5ccccc5-6)cc4)n3)cc21 ; O=S1(=O)c2ccccc2-c2ccc(-c3nc(-c4ccccc4)nc(-c4ccccc4-c4cccc5c4-c4ccccc4C54c5ccccc5-c5ccccc54)n3)cc21]

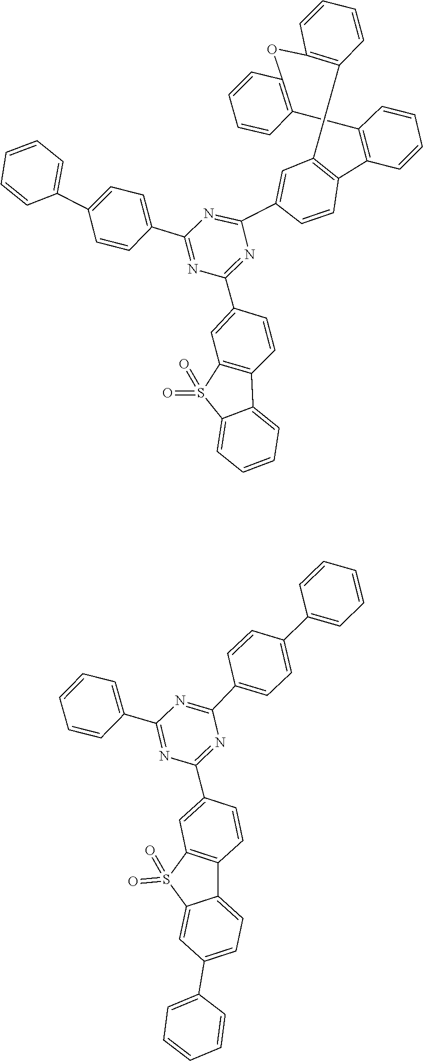 [SMILES: O=S1(=O)c2ccccc2-c2ccc(-c3nc(-c4ccc(-c5ccccc5)cc4)nc(-c4ccc5c(c4)-c4ccccc4Oc4ccccc4-c4ccccc4-5)n3)cc21 ; O=S1(=O)c2cc(-c3ccccc3)ccc2-c2ccc(-c3nc(-c4ccccc4)nc(-c4ccc(-c5ccccc5)cc4)n3)cc21]

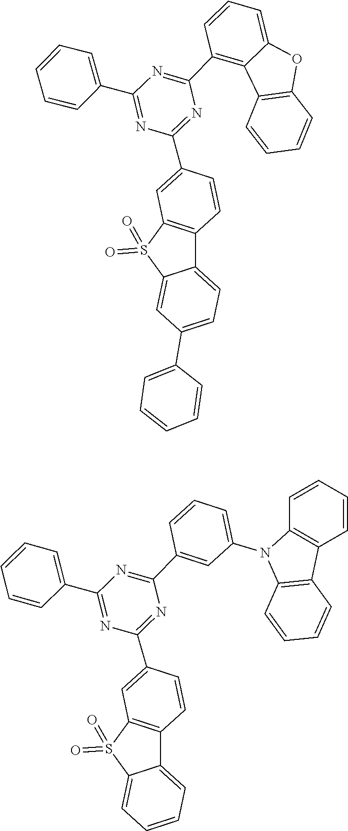 [SMILES: O=S1(=O)c2cc(-c3ccccc3)ccc2-c2ccc(-c3nc(-c4ccccc4)nc(-c4cccc5oc6ccccc6c45)n3)cc21 ; O=S1(=O)c2ccccc2-c2ccc(-c3nc(-c4ccccc4)nc(-c4cccc(-n5c6ccccc6c6ccccc65)c4)n3)cc21]

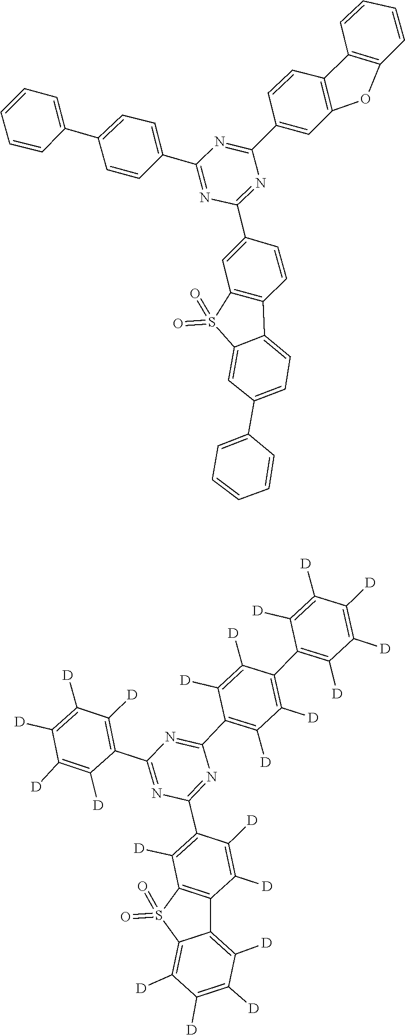 [SMILES: O=S1(=O)c2cc(-c3ccccc3)ccc2-c2ccc(-c3nc(-c4ccc(-c5ccccc5)cc4)nc(-c4ccc5c(c4)oc4ccccc45)n3)cc21 ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c4c(c3[2H])S(=O)(=O)c3c([2H])c([2H])c([2H])c([2H])c3-4)n2)c([2H])c1[2H]]

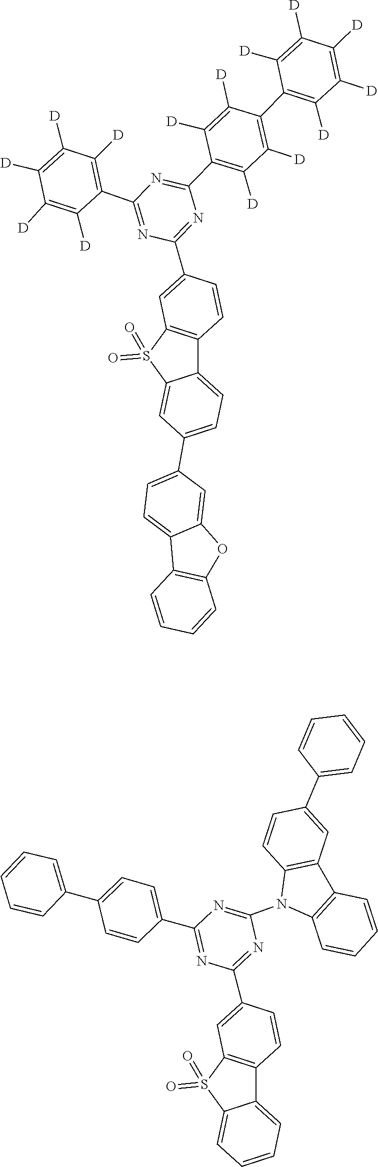 [SMILES: [2H]c1c([2H])c([2H])c(-c2nc(-c3ccc4c(c3)S(=O)(=O)c3cc(-c5ccc6c(c5)oc5ccccc56)ccc3-4)nc(-c3c([2H])c([2H])c(-c4c([2H])c([2H])c([2H])c([2H])c4[2H])c([2H])c3[2H])n2)c([2H])c1[2H] ; O=S1(=O)c2ccccc2-c2ccc(-c3nc(-c4ccc(-c5ccccc5)cc4)nc(-n4c5ccccc5c5cc(-c6ccccc6)ccc54)n3)cc21]

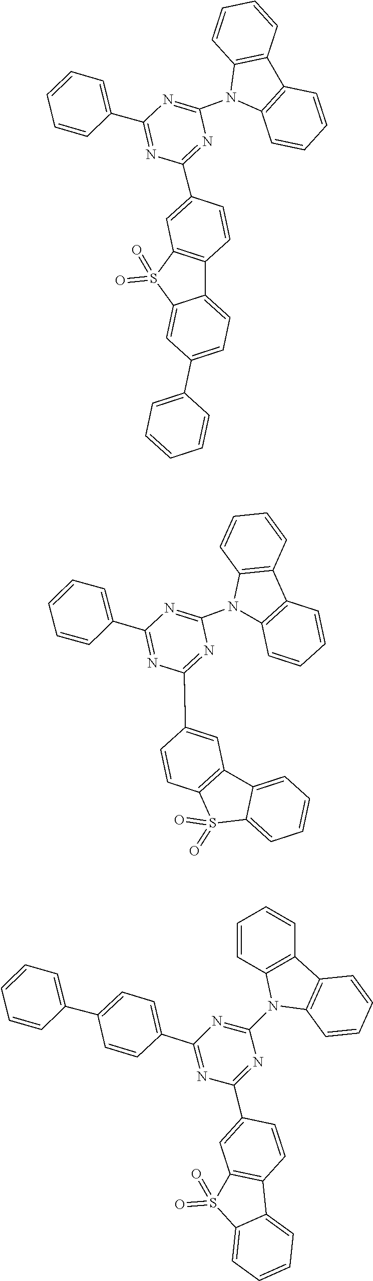 [SMILES: O=S1(=O)c2cc(-c3ccccc3)ccc2-c2ccc(-c3nc(-c4ccccc4)nc(-n4c5ccccc5c5ccccc54)n3)cc21 ; O=S1(=O)c2ccccc2-c2cc(-c3nc(-c4ccccc4)nc(-n4c5ccccc5c5ccccc54)n3)ccc21 ; O=S1(=O)c2ccccc2-c2ccc(-c3nc(-c4ccc(-c5ccccc5)cc4)nc(-n4c5ccccc5c5ccccc54)n3)cc21]

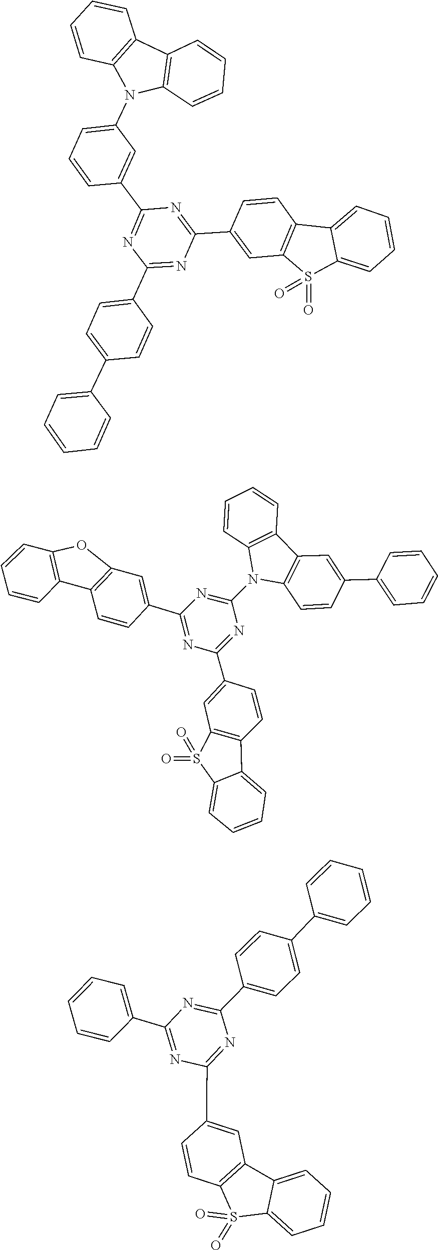 [SMILES: O=S1(=O)c2ccccc2-c2ccc(-c3nc(-c4ccc(-c5ccccc5)cc4)nc(-c4cccc(-n5c6ccccc6c6ccccc65)c4)n3)cc21 ; O=S1(=O)c2ccccc2-c2ccc(-c3nc(-c4ccc5c(c4)oc4ccccc45)nc(-n4c5ccccc5c5cc(-c6ccccc6)ccc54)n3)cc21 ; O=S1(=O)c2ccccc2-c2cc(-c3nc(-c4ccccc4)nc(-c4ccc(-c5ccccc5)cc4)n3)ccc21]

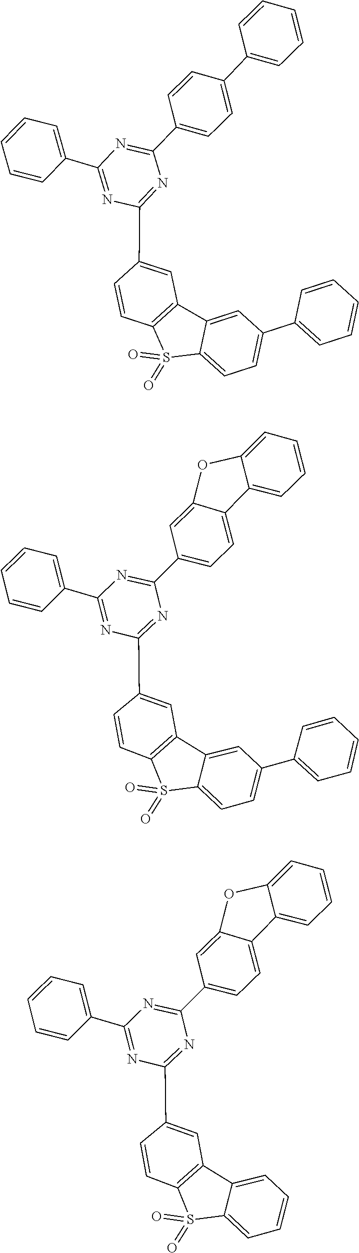 [SMILES: O=S1(=O)c2ccc(-c3ccccc3)cc2-c2cc(-c3nc(-c4ccccc4)nc(-c4ccc(-c5ccccc5)cc4)n3)ccc21 ; O=S1(=O)c2ccc(-c3ccccc3)cc2-c2cc(-c3nc(-c4ccccc4)nc(-c4ccc5c(c4)oc4ccccc45)n3)ccc21 ; O=S1(=O)c2ccccc2-c2cc(-c3nc(-c4ccccc4)nc(-c4ccc5c(c4)oc4ccccc45)n3)ccc21]

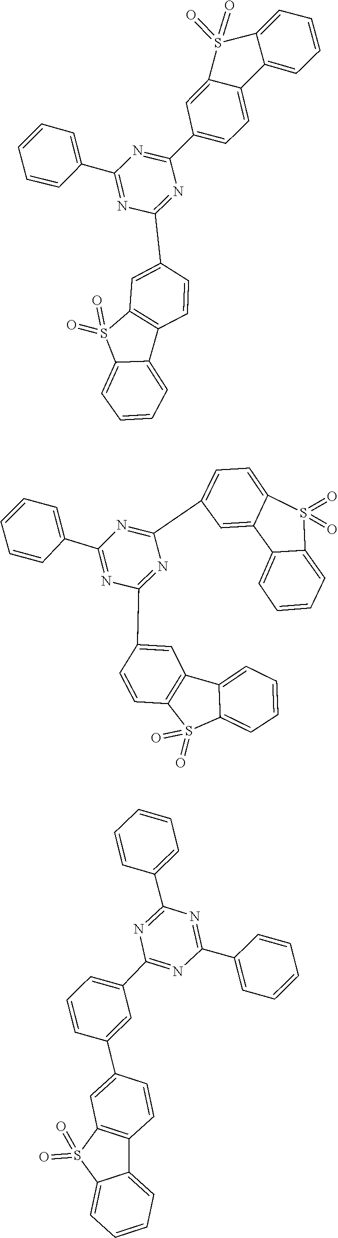 [SMILES: O=S1(=O)c2ccccc2-c2ccc(-c3nc(-c4ccccc4)nc(-c4ccc5c(c4)S(=O)(=O)c4ccccc4-5)n3)cc21 ; O=S1(=O)c2ccccc2-c2cc(-c3nc(-c4ccccc4)nc(-c4ccc5c(c4)-c4ccccc4S5(=O)=O)n3)ccc21 ; O=S1(=O)c2ccccc2-c2ccc(-c3cccc(-c4nc(-c5ccccc5)nc(-c5ccccc5)n4)c3)cc21]

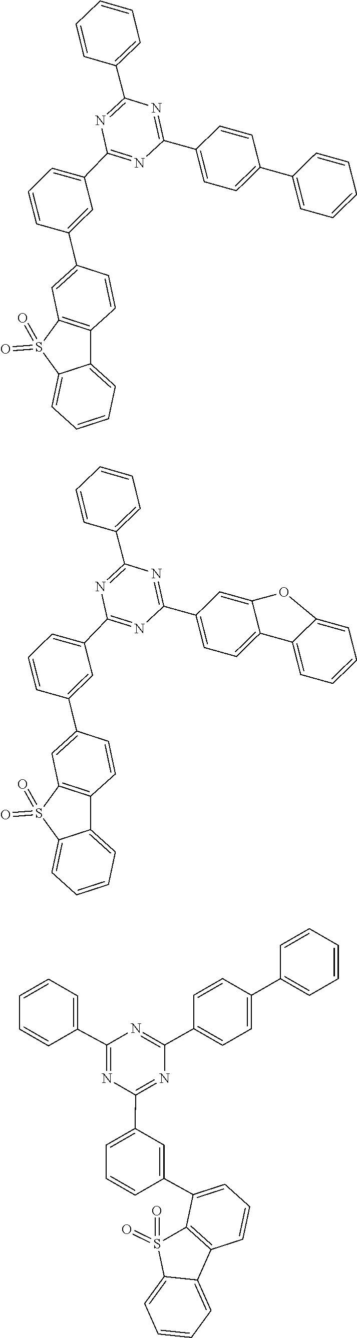

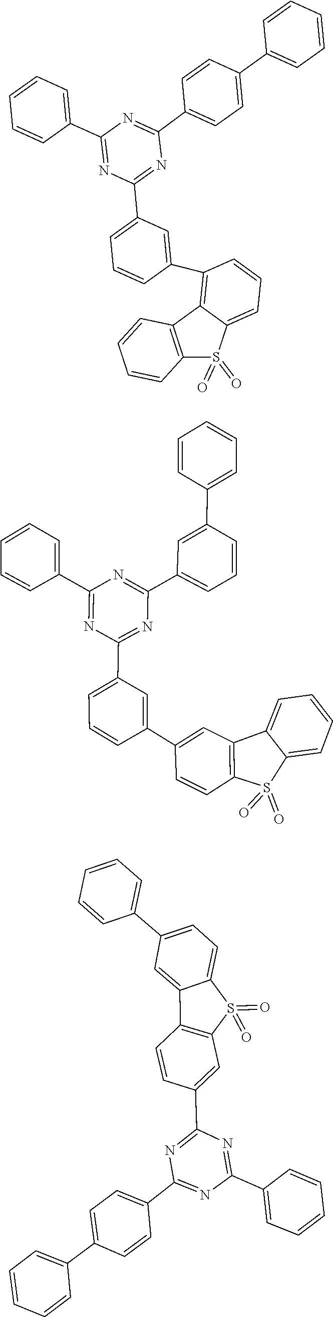

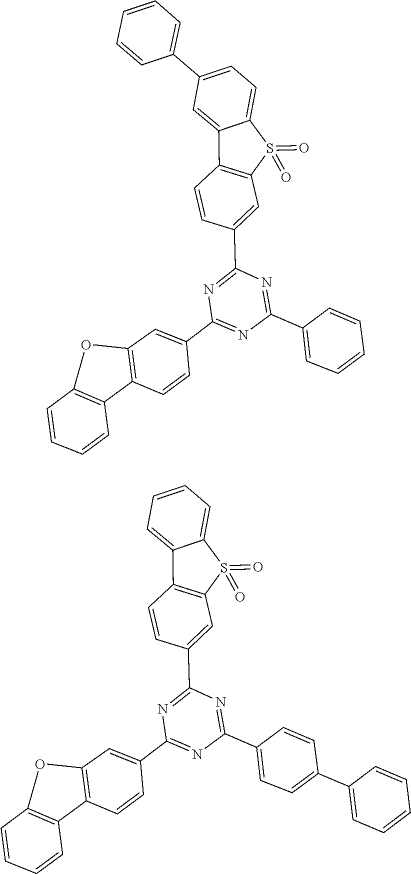 [SMILES: O=S1(=O)c2ccc(-c3ccccc3)cc2-c2ccc(-c3nc(-c4ccccc4)nc(-c4ccc5c(c4)oc4ccccc45)n3)cc21 ; O=S1(=O)c2ccccc2-c2ccc(-c3nc(-c4ccc(-c5ccccc5)cc4)nc(-c4ccc5c(c4)oc4ccccc45)n3)cc21]

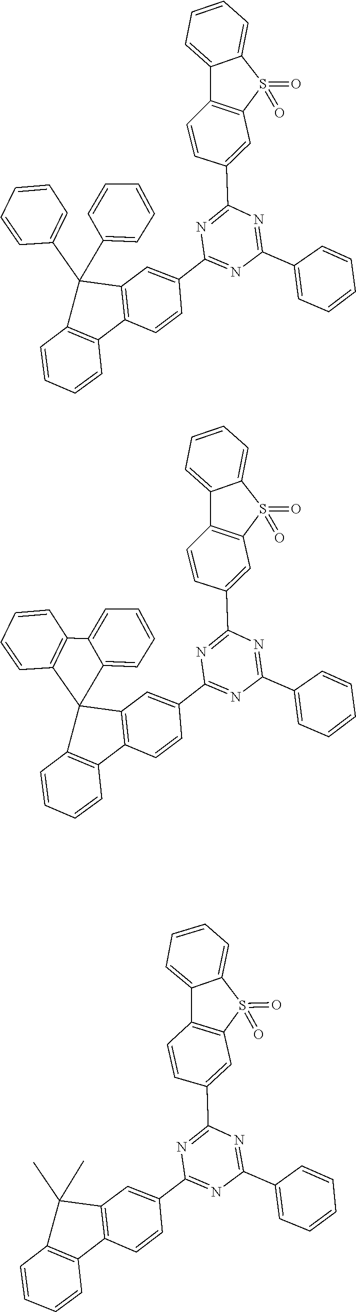 [SMILES: O=S1(=O)c2ccccc2-c2ccc(-c3nc(-c4ccccc4)nc(-c4ccc5c(c4)C(c4ccccc4)(c4ccccc4)c4ccccc4-5)n3)cc21 ; O=S1(=O)c2ccccc2-c2ccc(-c3nc(-c4ccccc4)nc(-c4ccc5c(c4)C4(c6ccccc6-c6ccccc64)c4ccccc4-5)n3)cc21 ; CC1(C)c2ccccc2-c2ccc(-c3nc(-c4ccccc4)nc(-c4ccc5c(c4)S(=O)(=O)c4ccccc4-5)n3)cc21]

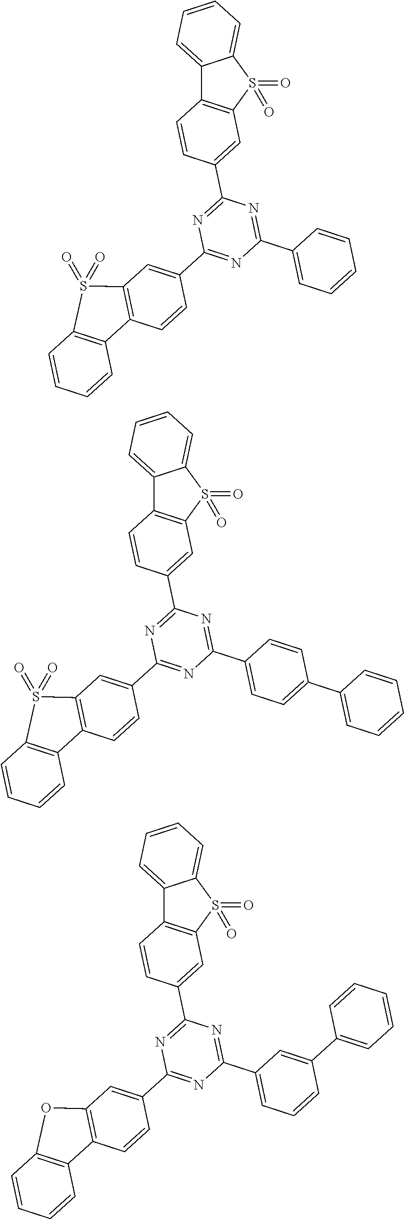 [SMILES: O=S1(=O)c2ccccc2-c2ccc(-c3nc(-c4ccccc4)nc(-c4ccc5c(c4)S(=O)(=O)c4ccccc4-5)n3)cc21 ; O=S1(=O)c2ccccc2-c2ccc(-c3nc(-c4ccc(-c5ccccc5)cc4)nc(-c4ccc5c(c4)S(=O)(=O)c4ccccc4-5)n3)cc21 ; O=S1(=O)c2ccccc2-c2ccc(-c3nc(-c4cccc(-c5ccccc5)c4)nc(-c4ccc5c(c4)oc4ccccc45)n3)cc21]

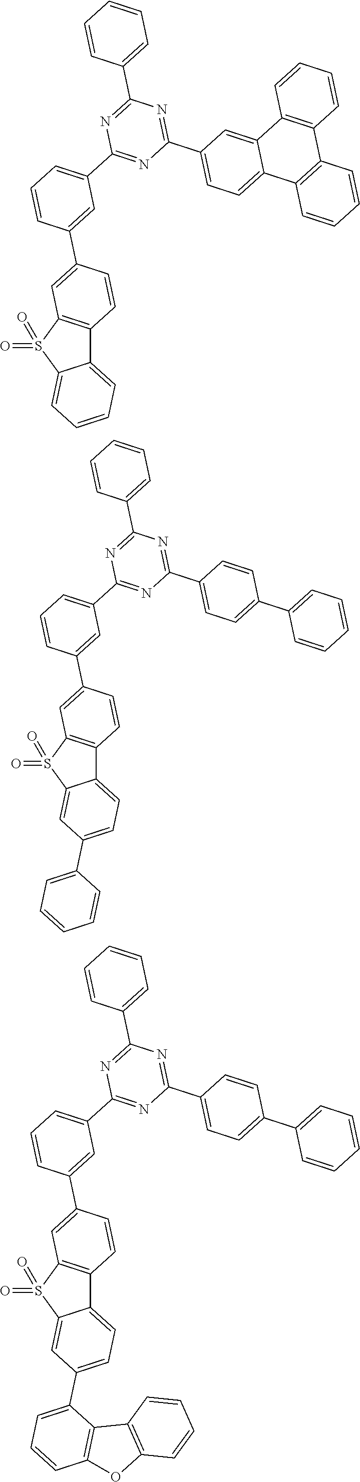

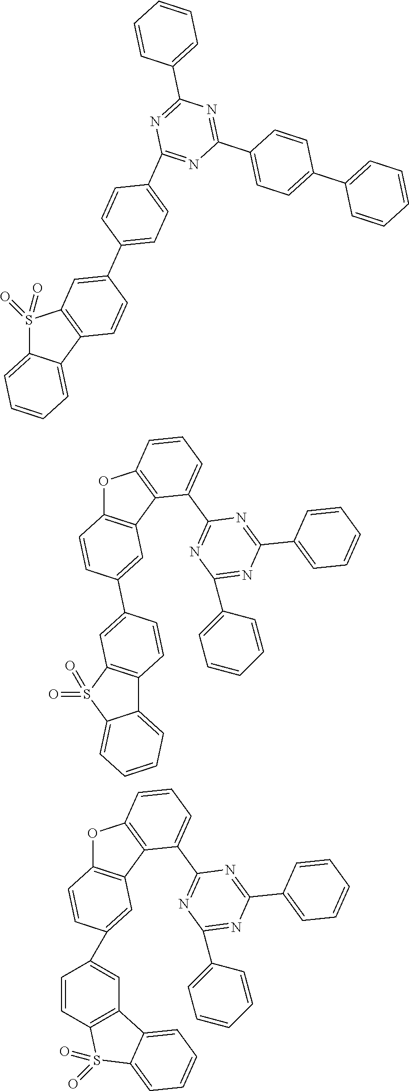

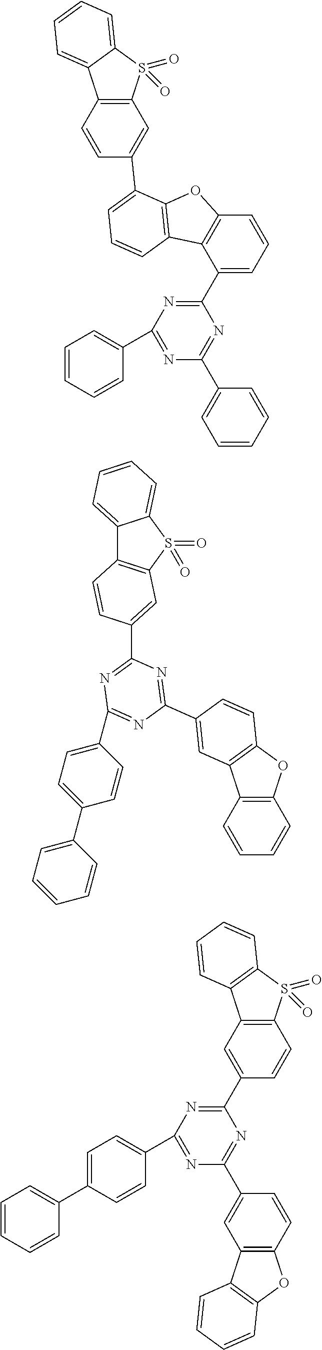 [SMILES: O=S1(=O)c2ccccc2-c2ccc(-c3cccc4c3oc3cccc(-c5nc(-c6ccccc6)nc(-c6ccccc6)n5)c34)cc21 ; O=S1(=O)c2ccccc2-c2ccc(-c3nc(-c4ccc(-c5ccccc5)cc4)nc(-c4ccc5oc6ccccc6c5c4)n3)cc21 ; O=S1(=O)c2ccccc2-c2cc(-c3nc(-c4ccc(-c5ccccc5)cc4)nc(-c4ccc5oc6ccccc6c5c4)n3)ccc21]

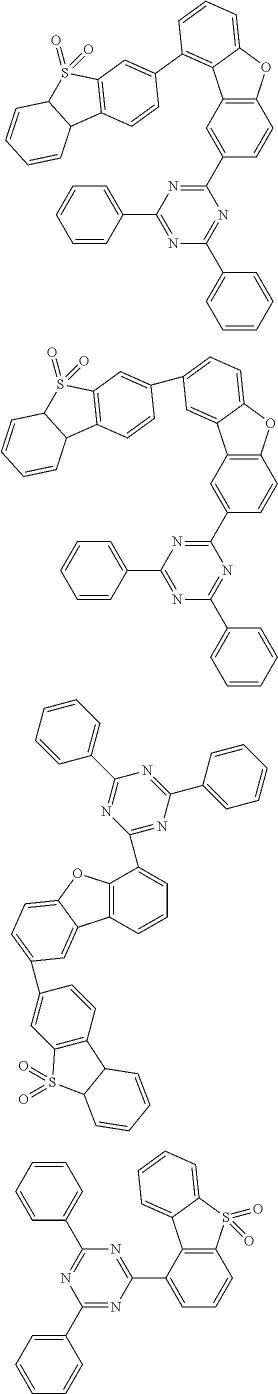

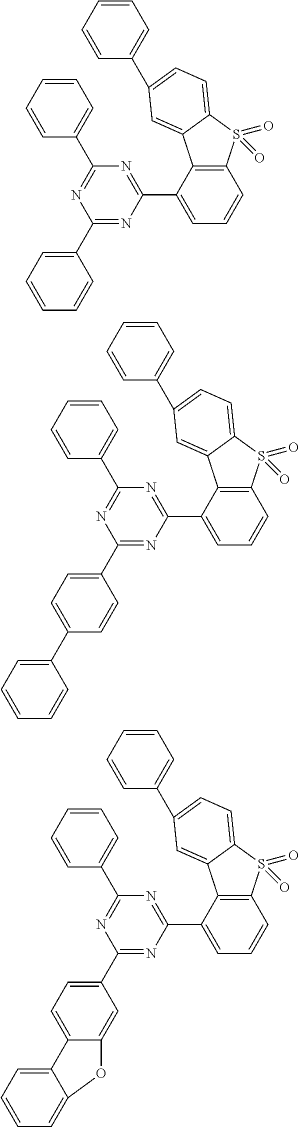 [SMILES: O=S1(=O)c2ccc(-c3ccccc3)cc2-c2c(-c3nc(-c4ccccc4)nc(-c4ccccc4)n3)cccc21 ; O=S1(=O)c2ccc(-c3ccccc3)cc2-c2c(-c3nc(-c4ccccc4)nc(-c4ccc(-c5ccccc5)cc4)n3)cccc21 ; O=S1(=O)c2ccc(-c3ccccc3)cc2-c2c(-c3nc(-c4ccccc4)nc(-c4ccc5c(c4)oc4ccccc45)n3)cccc21]

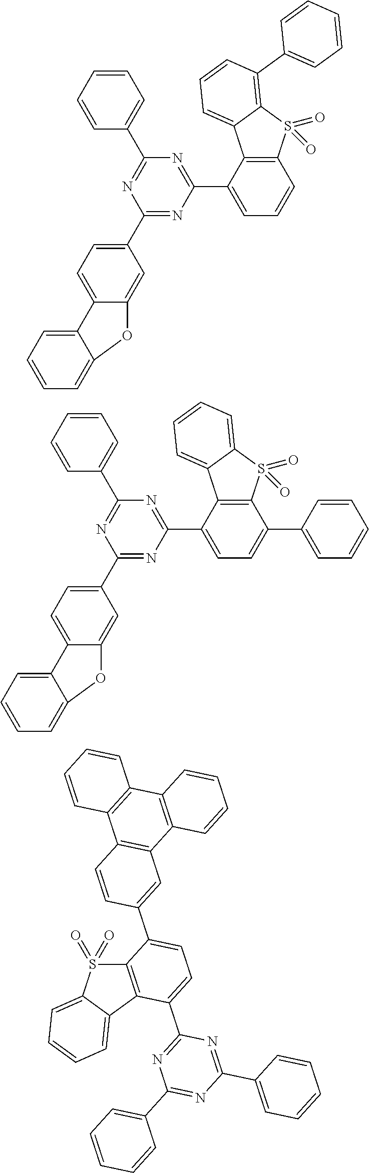

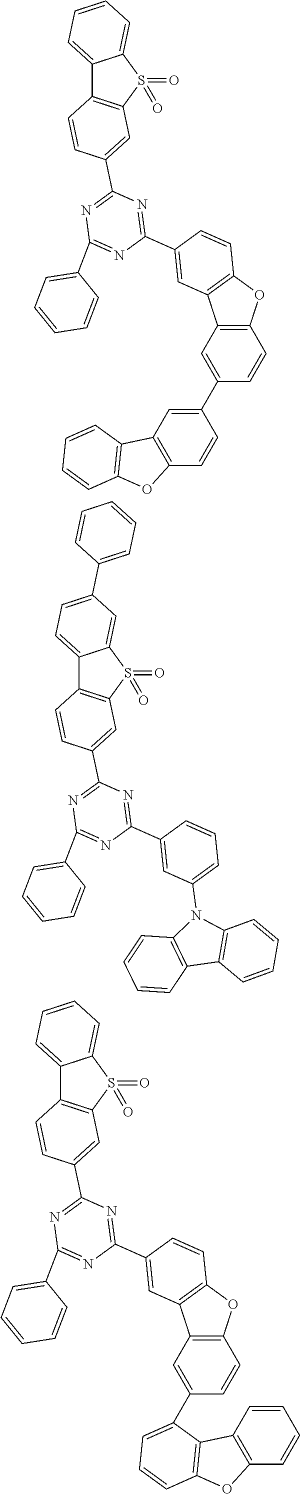

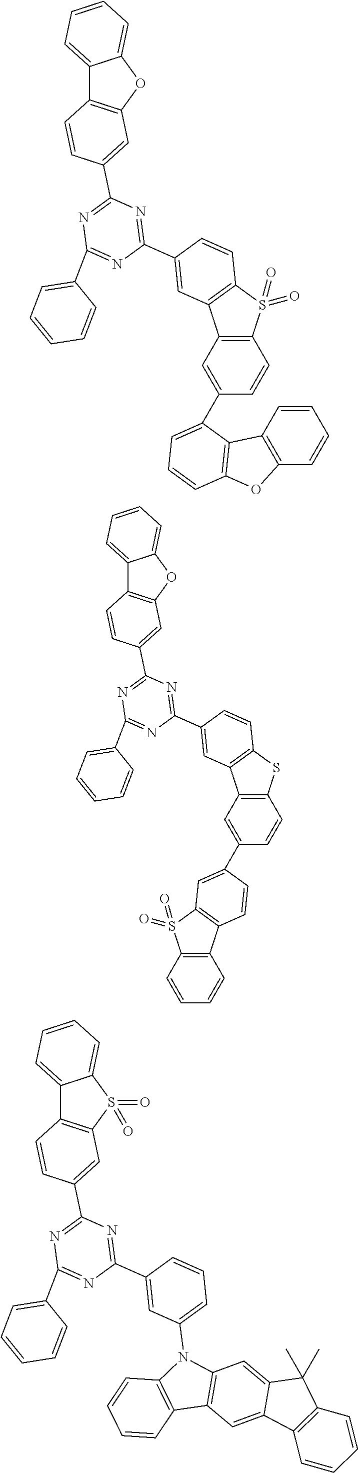 [SMILES: O=S1(=O)c2ccc(-c3nc(-c4ccccc4)nc(-c4ccc5c(c4)oc4ccccc45)n3)cc2-c2cc(-c3cccc4oc5ccccc5c34)ccc21 ; O=S1(=O)c2ccccc2-c2ccc(-c3ccc4sc5ccc(-c6nc(-c7ccccc7)nc(-c7ccc8c(c7)oc7ccccc78)n6)cc5c4c3)cc21 ; CC1(C)c2ccccc2-c2cc3c4ccccc4n(-c4cccc(-c5nc(-c6ccccc6)nc(-c6ccc7c(c6)S(=O)(=O)c6ccccc6-7)n5)c4)c3cc21]

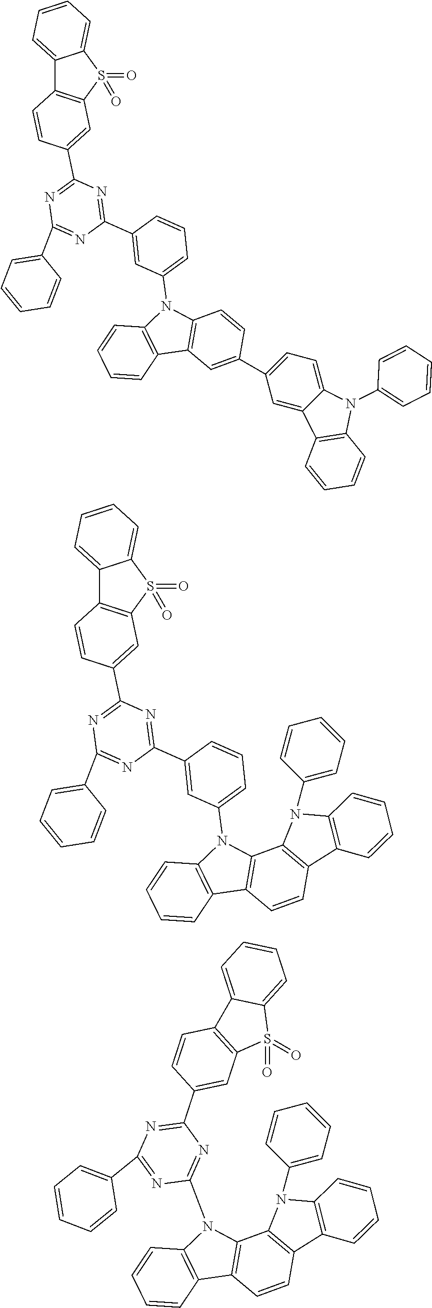

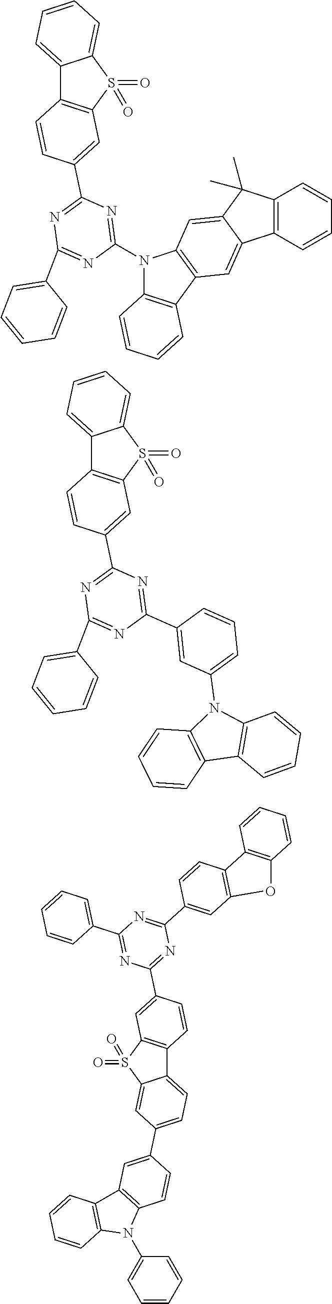

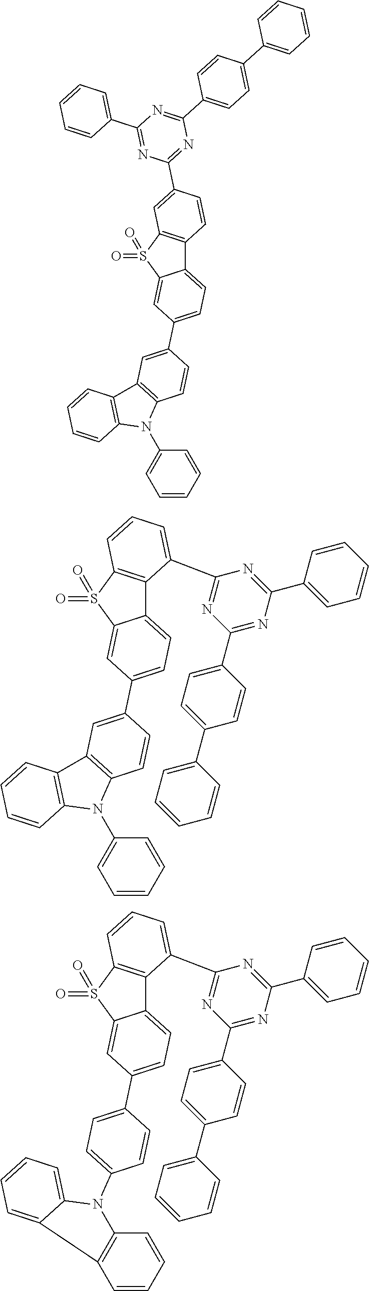

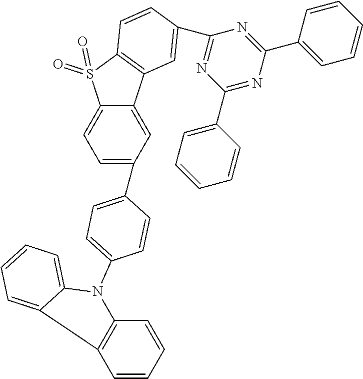 [SMILES: O=S1(=O)c2ccc(-c3ccc(-n4c5ccccc5c5ccccc54)cc3)cc2-c2cc(-c3nc(-c4ccccc4)nc(-c4ccccc4)n3)ccc21]

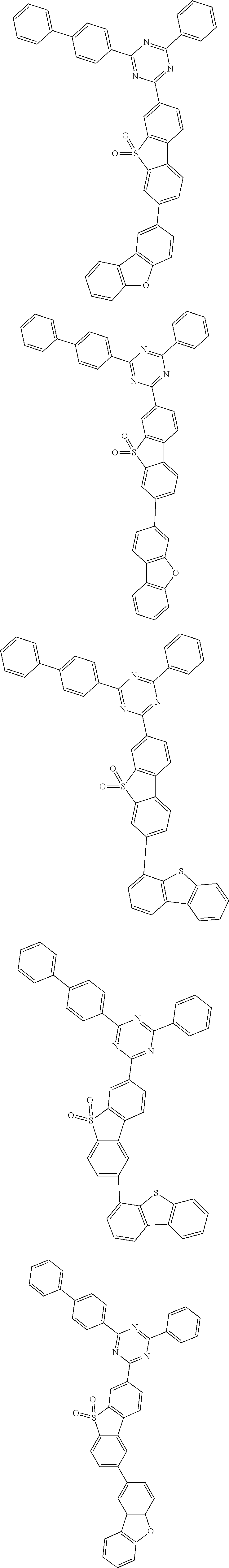

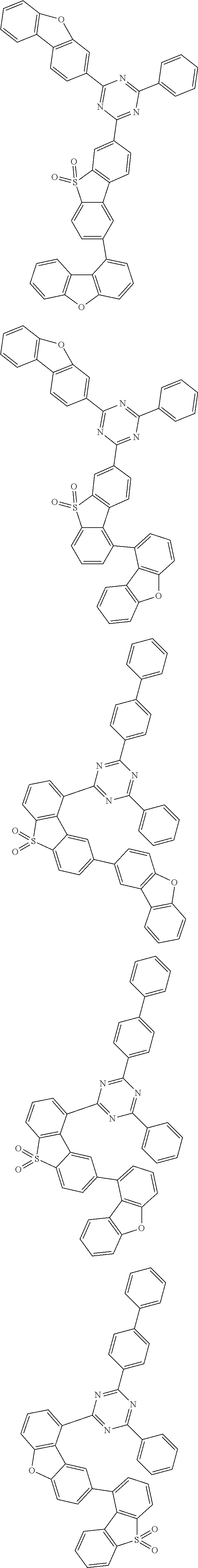

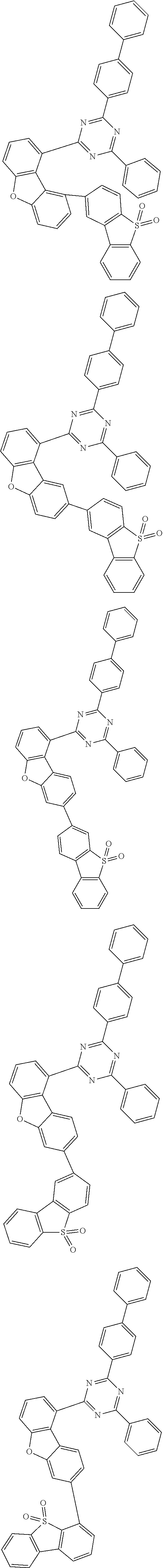

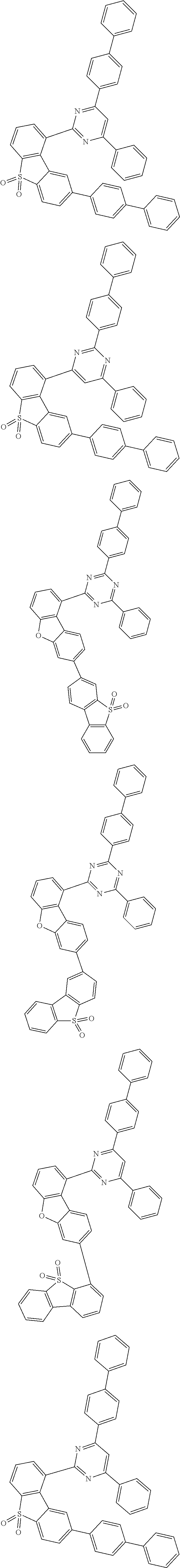

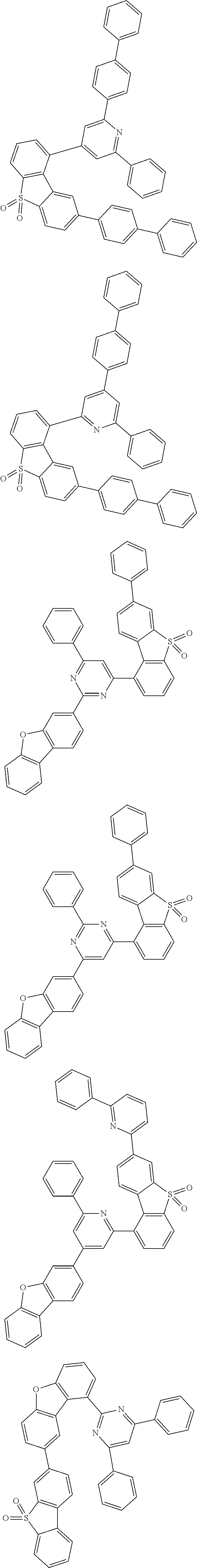

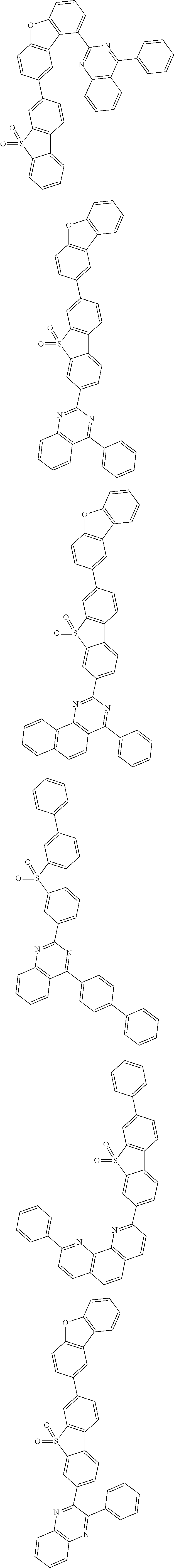

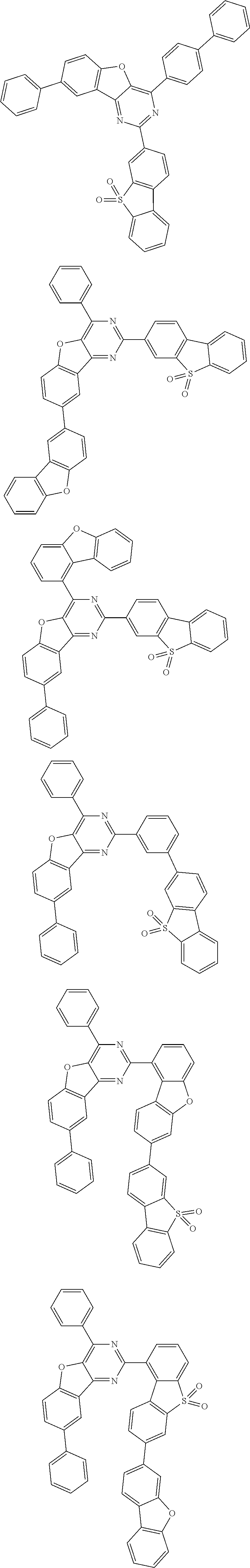

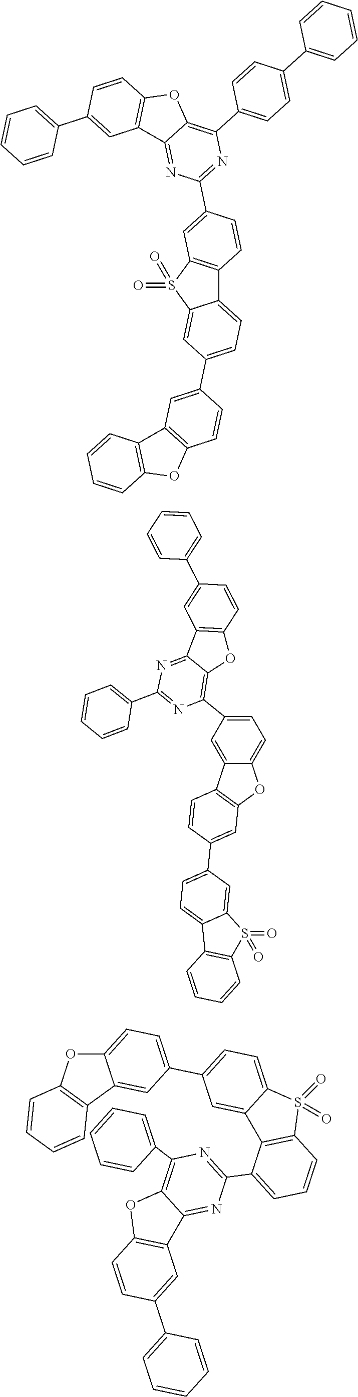

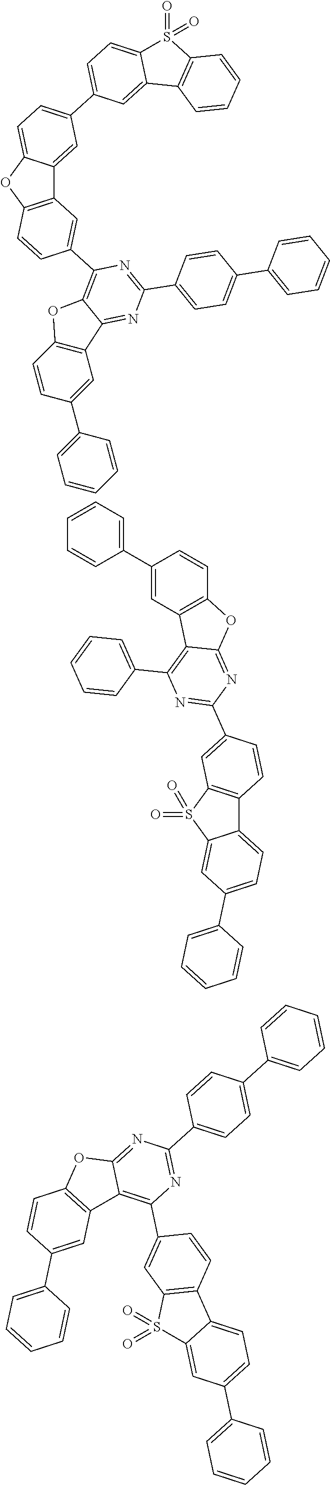

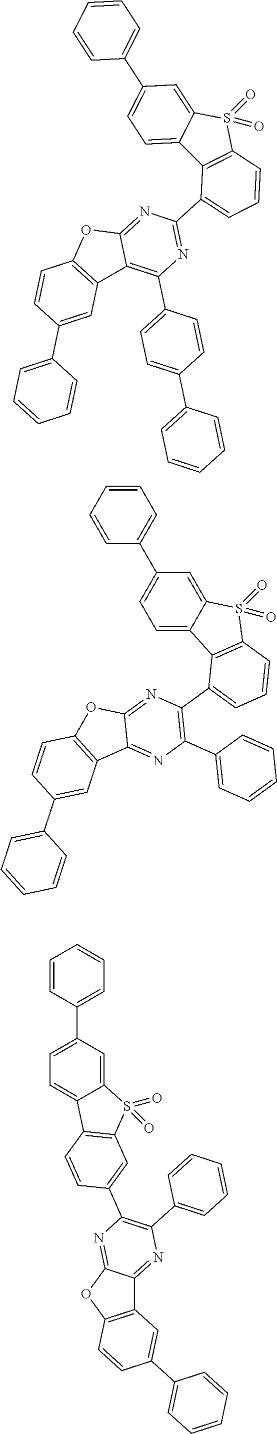

The base structure of the compounds of the invention is known in the literature. These can be prepared and functionalized by the routes outlined in schemes 1 and 2 that follow.

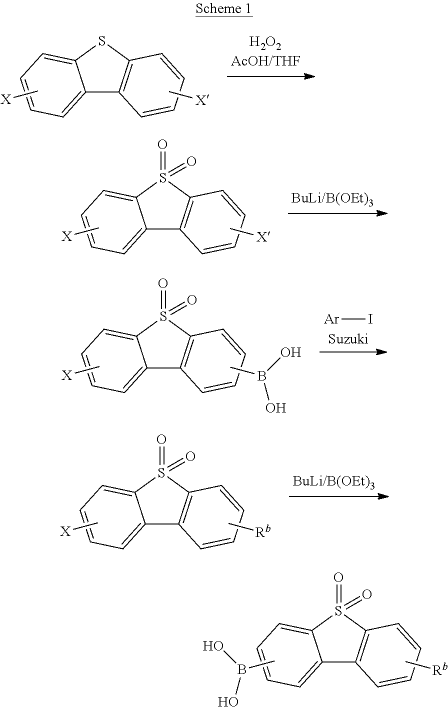

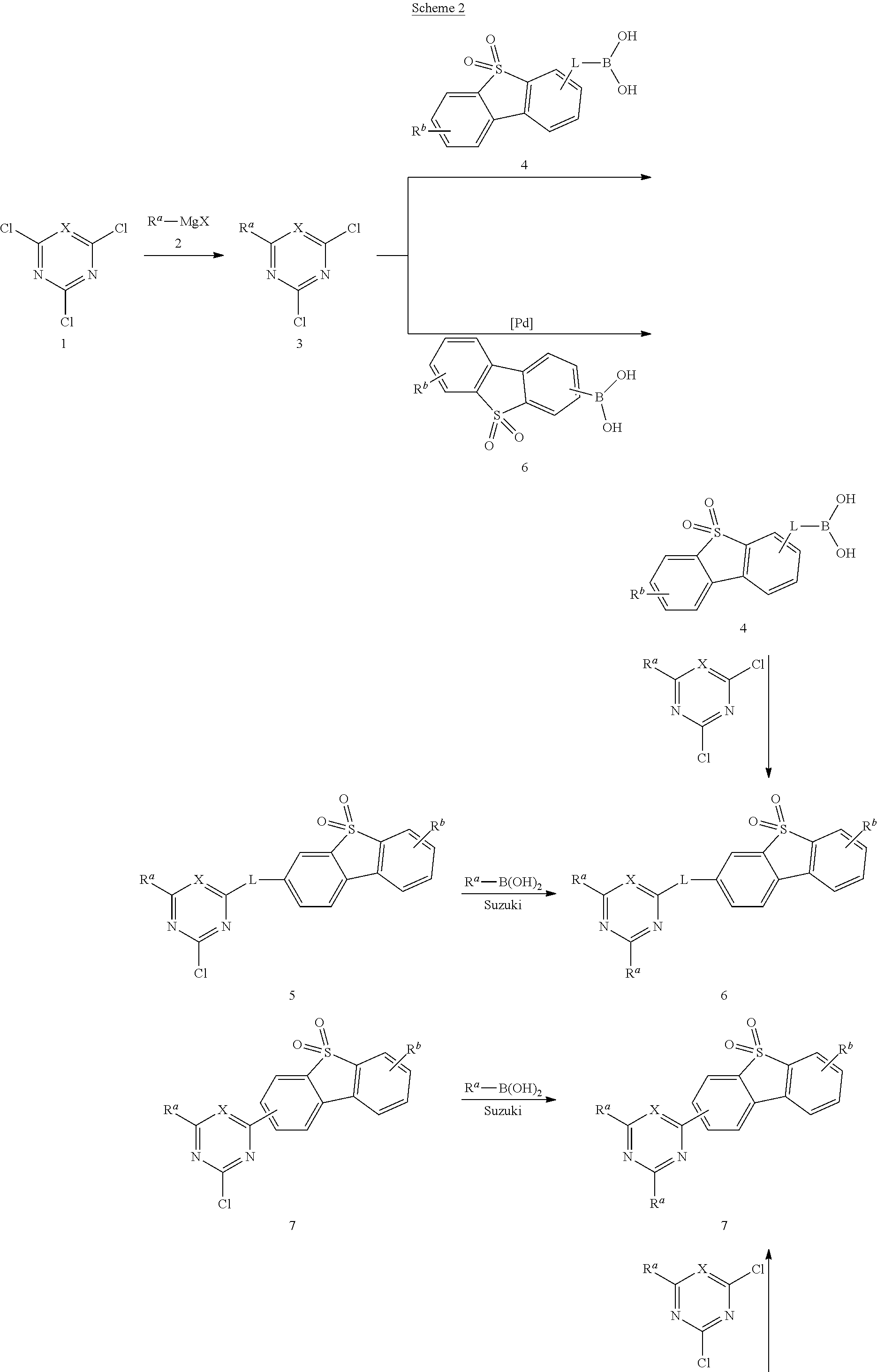

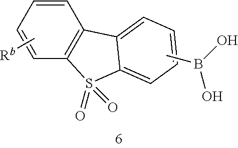

The present invention therefore further provides a process for preparing the compounds of the invention, characterized by the following steps:
(a) synthesizing the dibenzothiophene dioxide base skeleton that bears, in place of the substituent R*, a reactive leaving group, especially Cl, Br, I, triflate or a boronic acid or boronic ester derivative, and
(b) introducing the substituent R* by a coupling reaction.

For the processing of the compounds of formula (1) or the preferred embodiments from the liquid phase, for example by spin-coating or by printing methods, formulations of the compounds of the invention are required. The present invention therefore further provides formulations comprising at least one compound of formula (1) or the preferred embodiments and at least one solvent. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The compounds of the formula (1) or the above-detailed preferred embodiments are used in accordance with the invention in an electronic device, especially in an organic electroluminescent device. The present invention therefore further provides for the use of the compounds of formula (1) or the preferred embodiments in an electronic device, especially in an OLED.

The present invention still further provides an electronic device, especially an organic electroluminescent device, comprising at least one compound of the invention. An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound according to the above-detailed embodiments may be used in different layers, according to the exact structure. Preference is given to an organic electroluminescent device comprising a compound of formula (1) or the above-recited preferred embodiments in an emitting layer as matrix material for phosphorescent or fluorescent emitters or for emitters that exhibit TADF (thermally activated delayed fluorescence), especially as matrix material for phosphorescent emitters. In this case, the organic electroluminescent device may contain an emitting layer, or it may contain a plurality of emitting layers, where at least one emitting layer contains at least one compound of the invention as matrix material. In addition, the compound of the invention may also be used in an electron transport layer and/or in a hole blocker layer.

When the compound is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the formula (1) or of the preferred embodiments and the emitting compound contains between 99% and 1% by volume, preferably between 98% and 10% by volume, more preferably between 97% and 60% by volume and especially between 95% and 80% by volume of the compound of the formula (1) or of the preferred embodiments, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the formula (1) or of the preferred embodiments as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or those in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

In a preferred embodiment of the invention, the materials are used in combination with a further matrix material. The compounds of the formula (1) or the preferred embodiments are electron-deficient compounds. Preferred co-matrix materials are therefore hole-transporting compounds that are preferably selected from the group of the arylamine or carbazole derivatives.

Depicted below are examples of compounds that are suitable as co-matrix materials together with the compounds of the invention.

Preferred biscarbazoles are the structures of the following formulae (22) to (28):

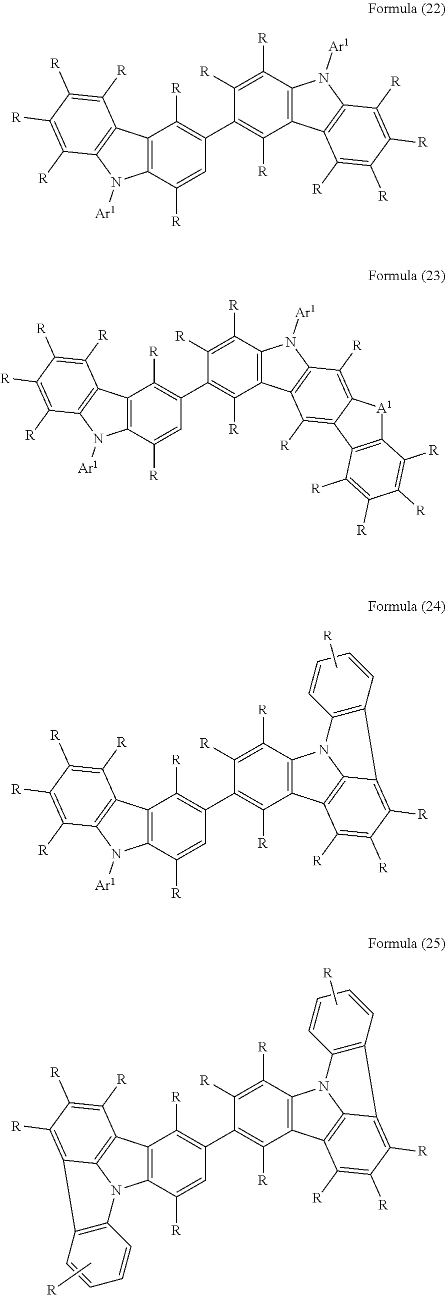

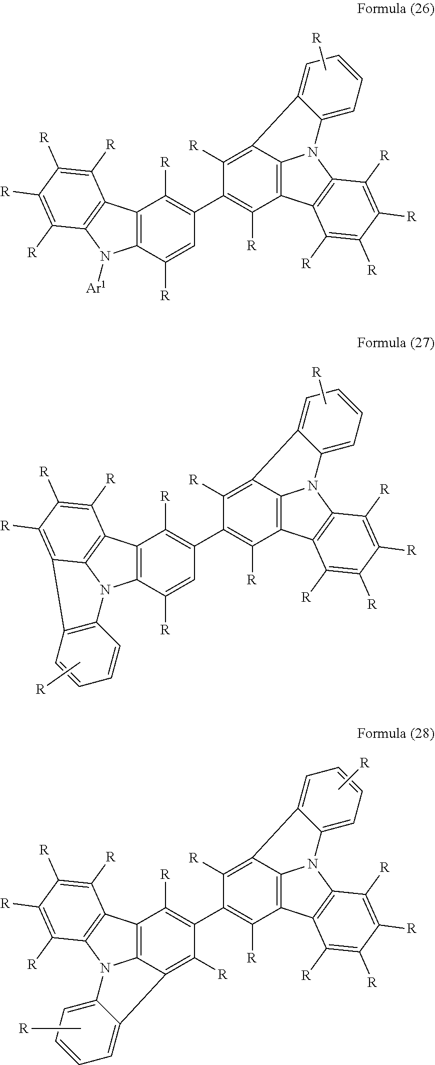

where A¹ has the definitions given above and Ar¹ is the same or different at each instance and is selected from an aromatic or heteroaromatic ring system which has 5 to 40 aromatic ring atoms and may be substituted by one or more R¹ radicals. In a preferred embodiment of the invention, A¹ is NR¹ or C(R¹)₂. Preferred embodiments of R¹ are the embodiments of R¹ that are given above in the definition of A¹. Preferred embodiments of Ar¹ are the preferred structures listed above for aromatic or heteroaromatic R radicals, especially the groups (R-1) to (R-83).

Preferred embodiments of the compounds of the formulae (22) to (28) are the compounds of the following formulae (22a) to (28a):

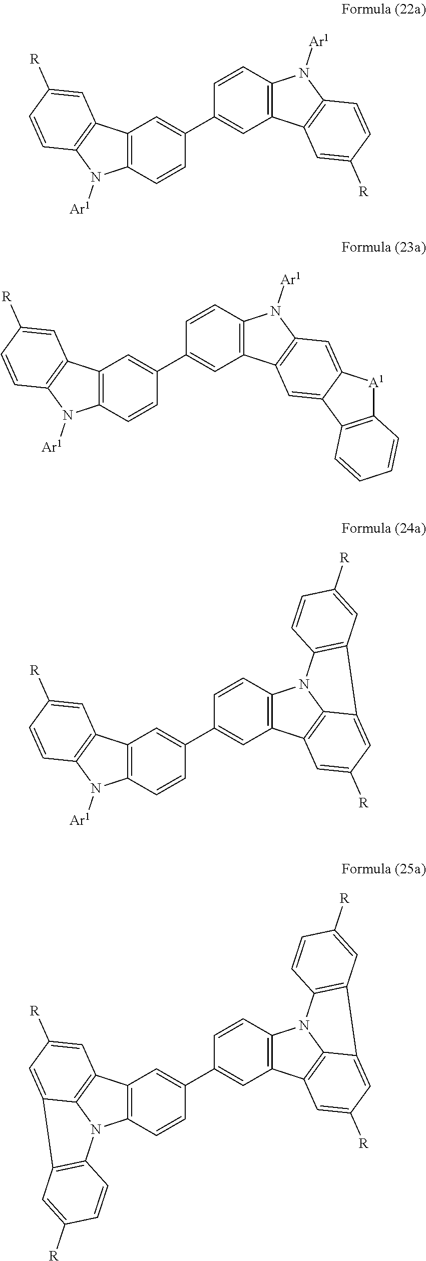

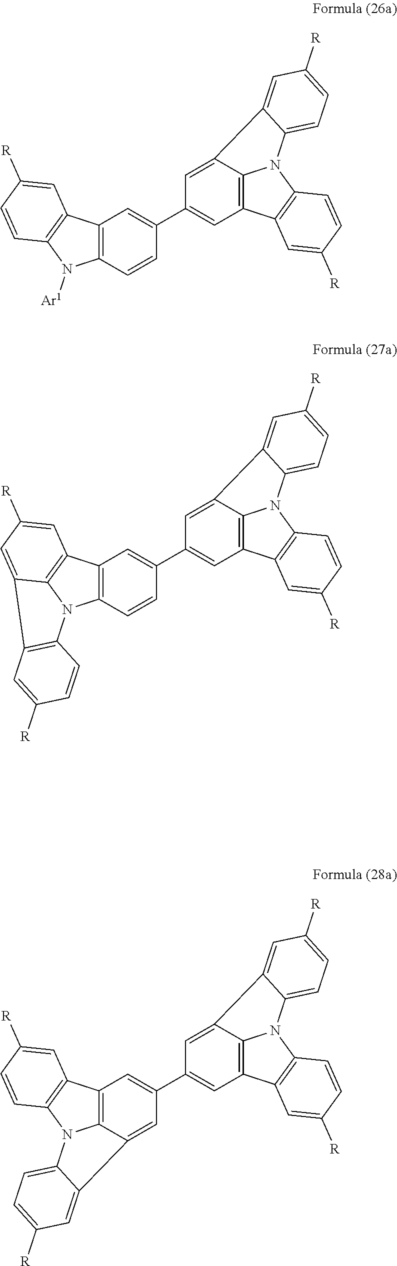

where the symbols used have the definitions given above.

Examples of suitable compounds of formulae (22) to (28) are the compounds depicted below:

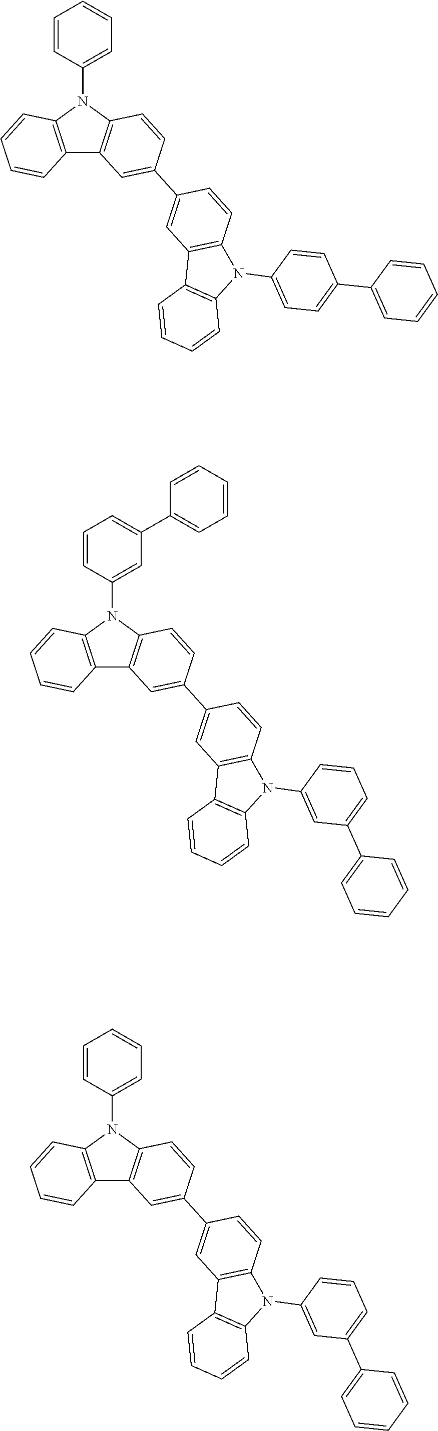 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1]

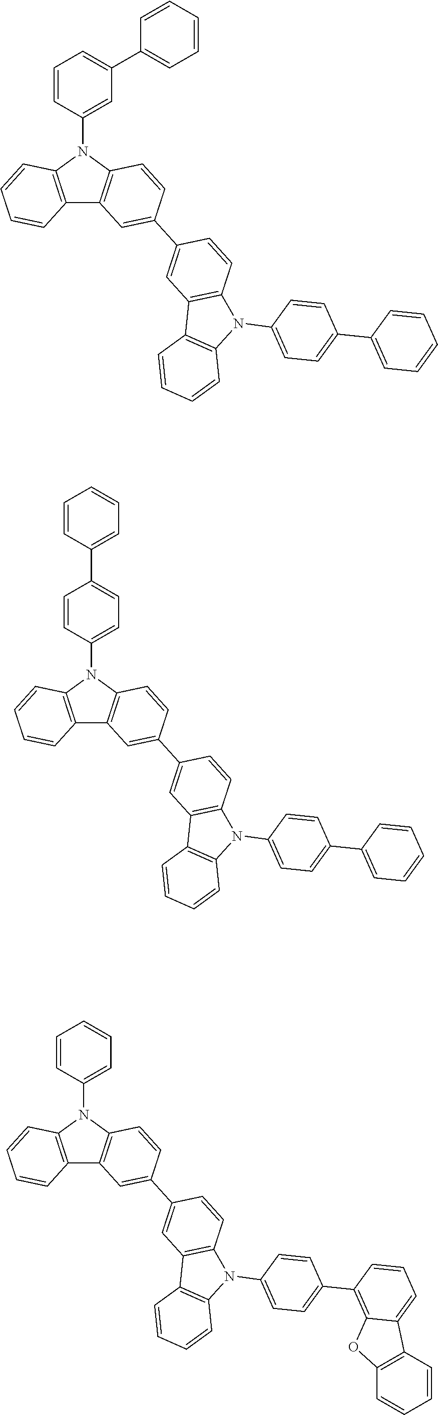 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

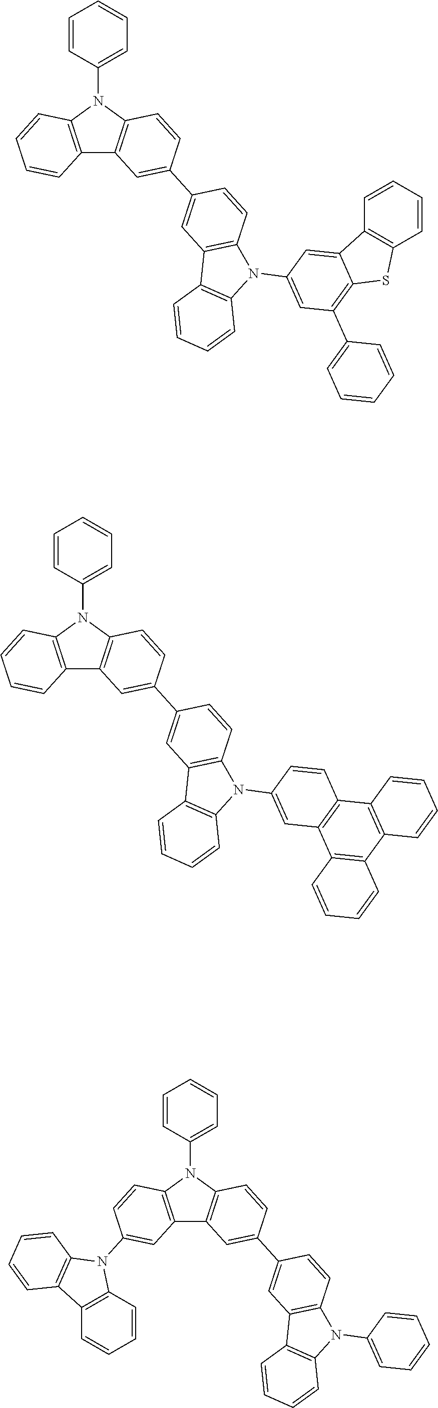 [SMILES: c1ccc(-c2cc(-n3c4ccccc4c4cc(-c5ccc6c(c5)c5ccccc5n6-c5ccccc5)ccc43)cc3c2sc2ccccc23)cc1 ; c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4cc(-n6c7ccccc7c7ccccc76)ccc4n5-c4ccccc4)ccc32)cc1]

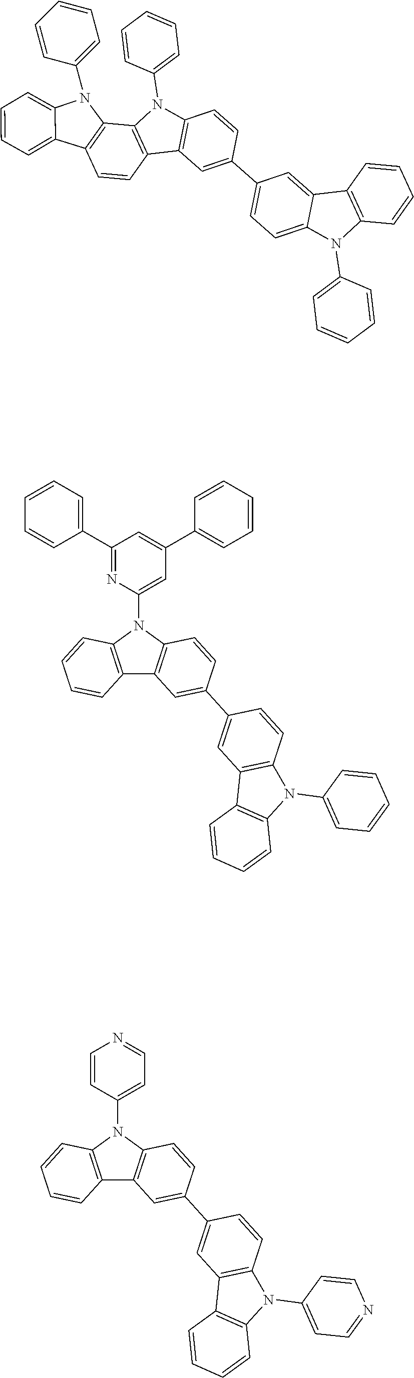 [SMILES: c1ccc(-n2c3ccccc3c3cc(-c4ccc5c(c4)c4ccc6c7ccccc7n(-c7ccccc7)c6c4n5-c4ccccc4)ccc32)cc1 ; c1ccc(-c2cc(-c3ccccc3)nc(-n3c4ccccc4c4cc(-c5ccc6c(c5)c5ccccc5n6-c5ccccc5)ccc43)c2)cc1 ; c1ccc2c(c1)c1cc(-c3ccc4c(c3)c3ccccc3n4-c3ccncc3)ccc1n2-c1ccncc1]

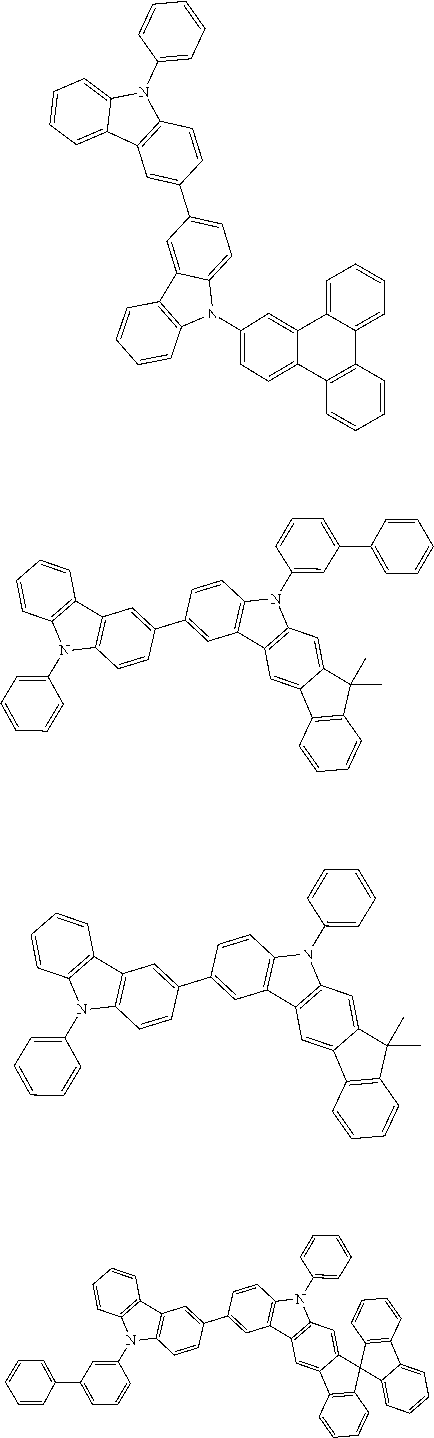 [SMILES: c1ccc(-n2c3ccccc3c3cc(-c4ccc5c(c4)c4ccccc4n5-c4ccc5c6ccccc6c6ccccc6c5c4)ccc32)cc1 ; CC1(C)c2ccccc2-c2cc3c4cc(-c5ccc6c(c5)c5ccccc5n6-c5ccccc5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4ccccc4)c3cc21 ; c1ccc(-c2cccc(-n3c4ccccc4c4cc(-c5ccc6c(c5)c5cc7c(cc5n6-c5ccccc5)C5(c6ccccc6-c6ccccc65)c5ccccc5-7)ccc43)c2)cc1]

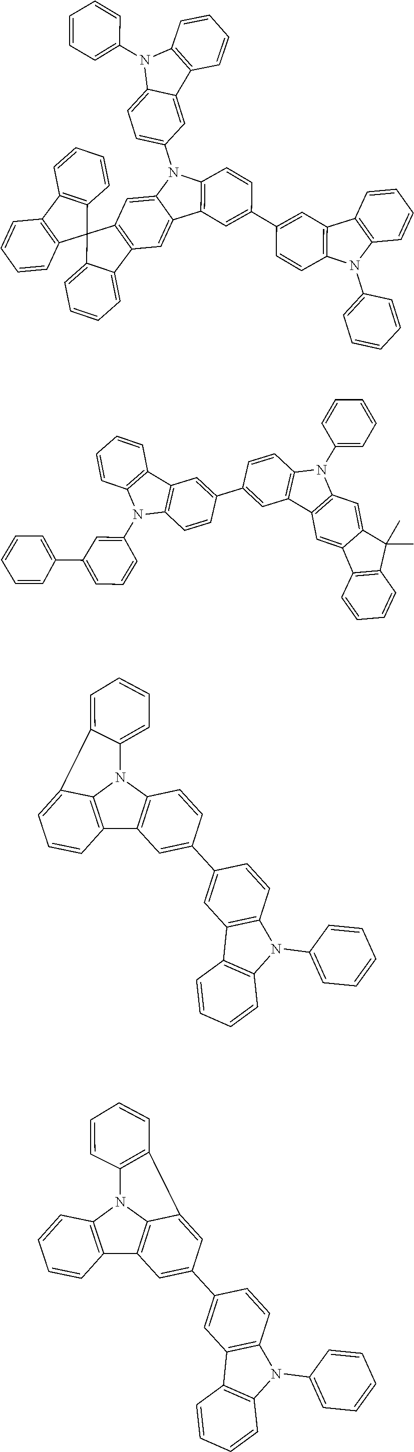 [SMILES: c1ccc(-n2c3ccccc3c3cc(-c4ccc5c(c4)c4cc6c(cc4n5-c4ccc5c(c4)c4ccccc4n5-c4ccccc4)C4(c5ccccc5-c5ccccc54)c4ccccc4-6)ccc32)cc1 ; CC1(C)c2ccccc2-c2cc3c4cc(-c5ccc6c(c5)c5ccccc5n6-c5cccc(-c6ccccc6)c5)ccc4n(-c4ccccc4)c3cc21 ; c1ccc(-n2c3ccccc3c3cc(-c4ccc5c(c4)c4cccc6c7ccccc7n5c64)ccc32)cc1 ; c1ccc(-n2c3ccccc3c3cc(-c4cc5c6ccccc6n6c7ccccc7c(c4)c56)ccc32)cc1]

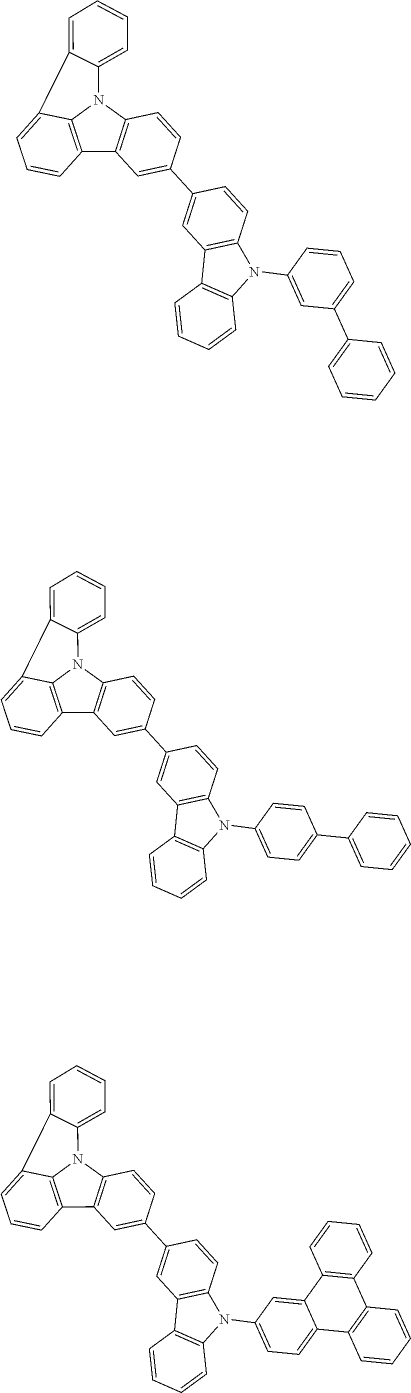 [SMILES: c1ccc(-c2cccc(-n3c4ccccc4c4cc(-c5ccc6c(c5)c5cccc7c8ccccc8n6c75)ccc43)c2)cc1 ; c1ccc(-c2ccc(-n3c4ccccc4c4cc(-c5ccc6c(c5)c5cccc7c8ccccc8n6c75)ccc43)cc2)cc1 ; c1ccc2c(c1)c1ccccc1c1cc(-n3c4ccccc4c4cc(-c5ccc6c(c5)c5cccc7c8ccccc8n6c75)ccc43)ccc21]

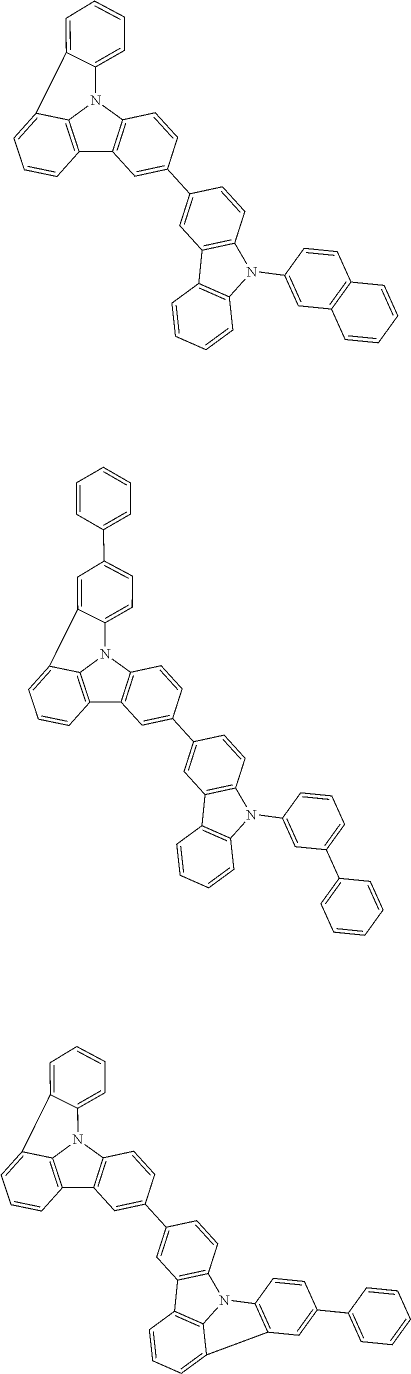 [SMILES: c1ccc2cc(-n3c4ccccc4c4cc(-c5ccc6c(c5)c5cccc7c8ccccc8n6c75)ccc43)ccc2c1 ; c1ccc(-c2cccc(-n3c4ccccc4c4cc(-c5ccc6c(c5)c5cccc7c8cc(-c9ccccc9)ccc8n6c75)ccc43)c2)cc1 ; c1ccc(-c2ccc3c(c2)c2cccc4c5cc(-c6ccc7c(c6)c6cccc8c9ccccc9n7c86)ccc5n3c24)cc1]

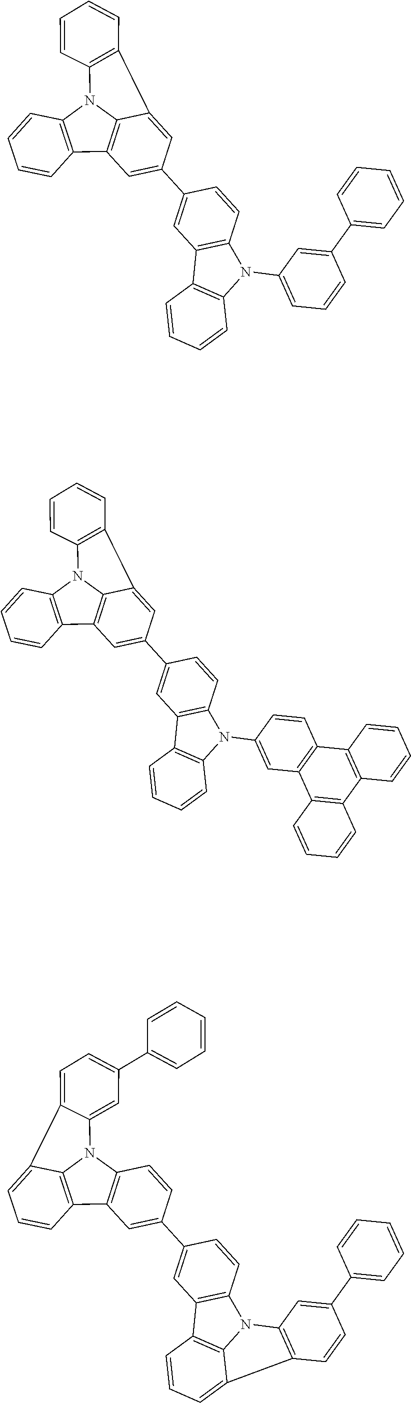 [SMILES: c1ccc(-c2cccc(-n3c4ccccc4c4cc(-c5cc6c7ccccc7n7c8ccccc8c(c5)c67)ccc43)c2)cc1 ; c1ccc2c(c1)c1ccccc1c1cc(-n3c4ccccc4c4cc(-c5cc6c7ccccc7n7c8ccccc8c(c5)c67)ccc43)ccc21 ; c1ccc(-c2ccc3c4cccc5c6cc(-c7ccc8c(c7)c7cccc9c%10ccc(-c%11ccccc%11)cc%10n8c79)ccc6n(c3c2)c54)cc1]

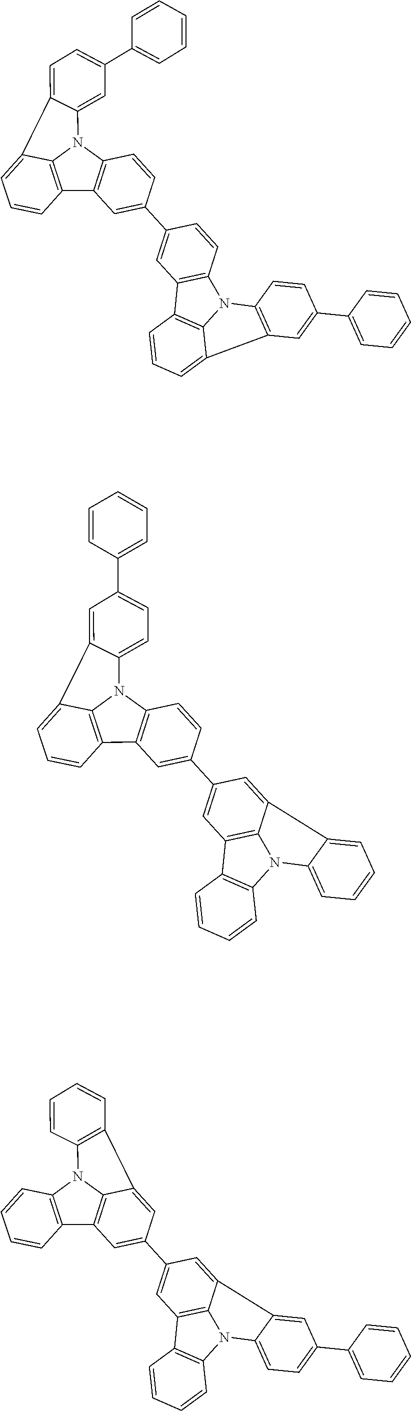 [SMILES: c1ccc(-c2ccc3c(c2)c2cccc4c5cc(-c6ccc7c(c6)c6cccc8c9ccc(-c%10ccccc%10)cc9n7c68)ccc5n3c24)cc1 ; c1ccc(-c2ccc3c(c2)c2cccc4c5cc(-c6cc7c8ccccc8n8c9ccccc9c(c6)c78)ccc5n3c24)cc1 ; c1ccc(-c2ccc3c(c2)c2cc(-c4cc5c6ccccc6n6c7ccccc7c(c4)c56)cc4c5ccccc5n3c42)cc1]

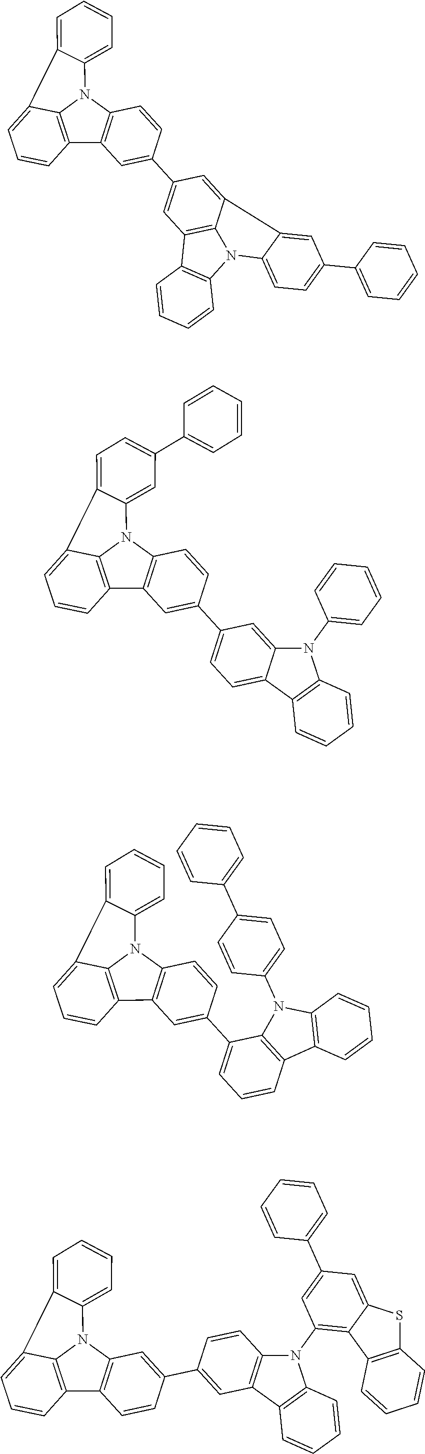 [SMILES: c1ccc(-c2ccc3c(c2)c2cc(-c4ccc5c(c4)c4cccc6c7ccccc7n5c64)cc4c5ccccc5n3c42)cc1 ; c1ccc(-c2ccc3c4cccc5c6cc(-c7ccc8c9ccccc9n(-c9ccccc9)c8c7)ccc6n(c3c2)c54)cc1 ; c1ccc(-c2ccc(-n3c4ccccc4c4cccc(-c5ccc6c(c5)c5cccc7c8ccccc8n6c75)c43)cc2)cc1 ; c1ccc(-c2cc(-n3c4ccccc4c4cc(-c5ccc6c7cccc8c9ccccc9n(c6c5)c87)ccc43)c3c(c2)sc2ccccc23)cc1]

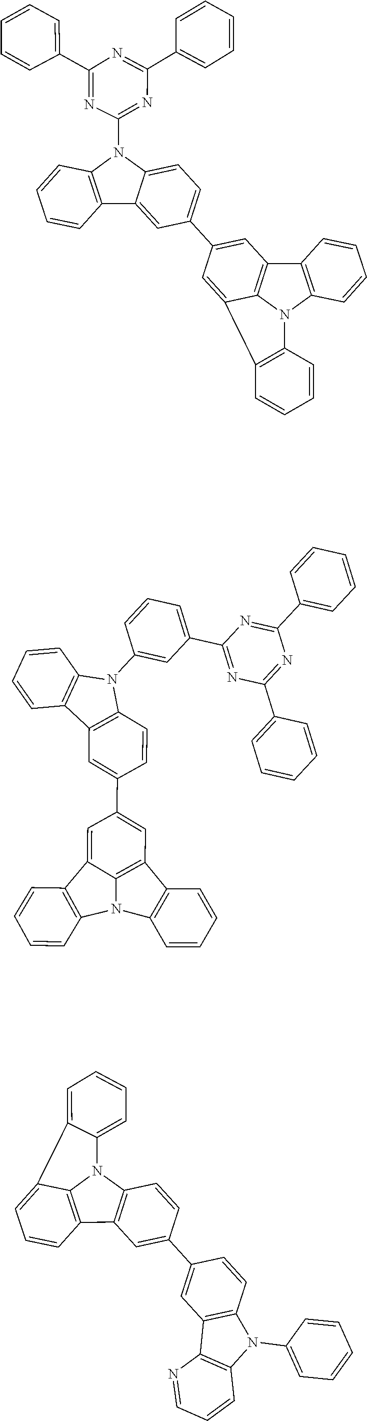 [SMILES: c1ccc(-c2nc(-c3ccccc3)nc(-n3c4ccccc4c4cc(-c5cc6c7ccccc7n7c8ccccc8c(c5)c67)ccc43)n2)cc1 ; c1ccc(-c2nc(-c3ccccc3)nc(-c3cccc(-n4c5ccccc5c5cc(-c6cc7c8ccccc8n8c9ccccc9c(c6)c78)ccc54)c3)n2)cc1 ; c1ccc(-n2c3ccc(-c4ccc5c(c4)c4cccc6c7ccccc7n5c64)cc3c3ncccc32)cc1]

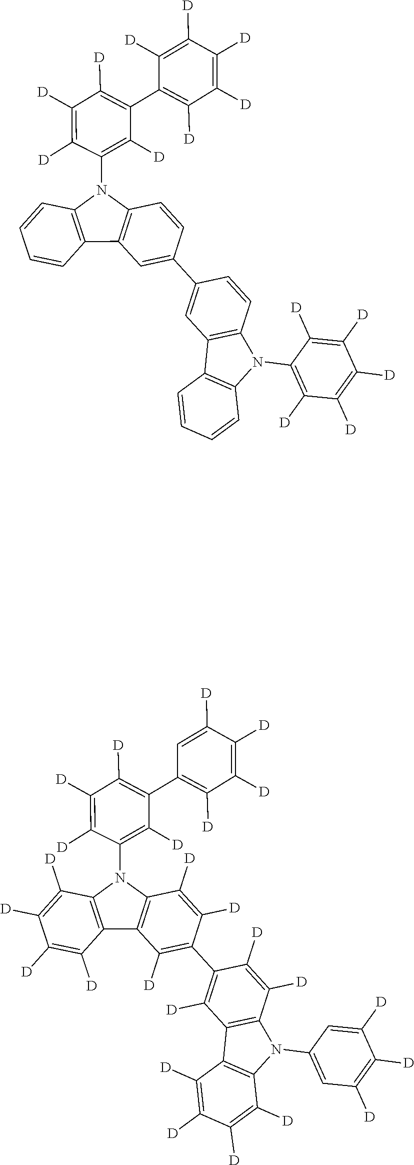 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H] ; [2H]c1cc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-c5cc([2H])c([2H])c([2H])c5[2H])c4[2H])c([2H])c([2H])c32)cc([2H])c1[2H]]

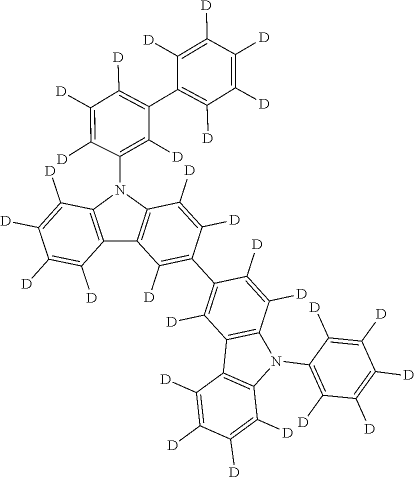 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]]

Preferred bridged carbazoles are the structures of the following formula (29):

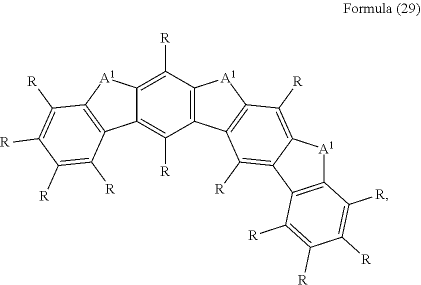

where A¹ and R have the definitions given above and A¹ is preferably the same or different at each instance and is selected from the group consisting of NR¹, O, S and C(R¹)₂, where R¹ is an aromatic or heteroaromatic ring system which has 5 to 24 aromatic ring atoms and may be substituted by one or more R² radicals.

Preferred dibenzofuran derivatives are the compounds of the following formula (30):

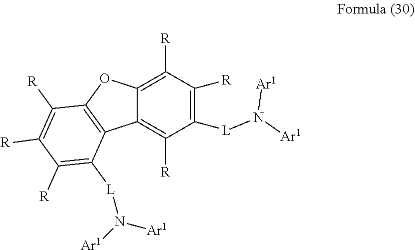

where the oxygen may also be replaced by sulfur, so as to form a dibenzothiophene, and L, R and Ar¹ have the definitions given above. It is also possible here for the two Ar¹ groups that bind to the same nitrogen atom, or for one Ar¹ group and one L group that bind to the same nitrogen atom, to be bonded to one another, for example to give a carbazole.

Examples of suitable dibenzofuran derivatives are the compounds depicted below.

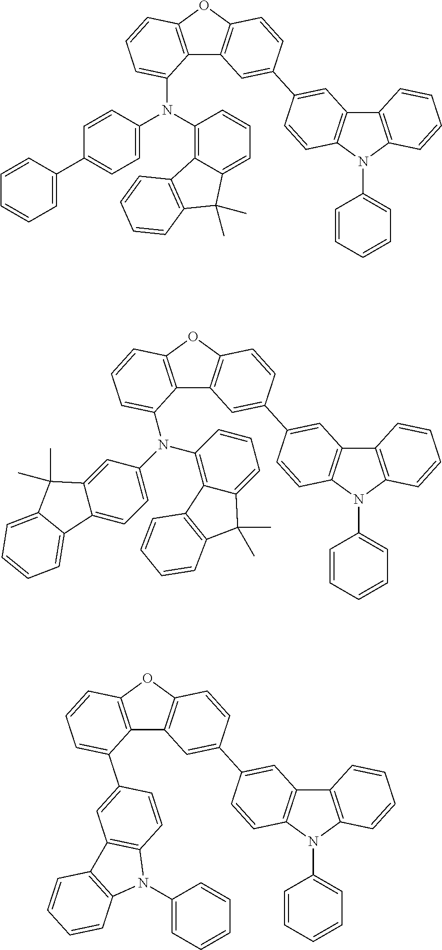 [SMILES: CC1(C)c2ccccc2-c2c(N(c3ccc(-c4ccccc4)cc3)c3cccc4oc5ccc(-c6ccc7c(c6)c6ccccc6n7-c6ccccc6)cc5c34)cccc21 ; CC1(C)c2ccccc2-c2ccc(N(c3cccc4c3-c3ccccc3C4(C)C)c3cccc4oc5ccc(-c6ccc7c(c6)c6ccccc6n7-c6ccccc6)cc5c34)cc21 ; c1ccc(-n2c3ccccc3c3cc(-c4ccc5oc6cccc(-c7ccc8c(c7)c7ccccc7n8-c7ccccc7)c6c5c4)ccc32)cc1]

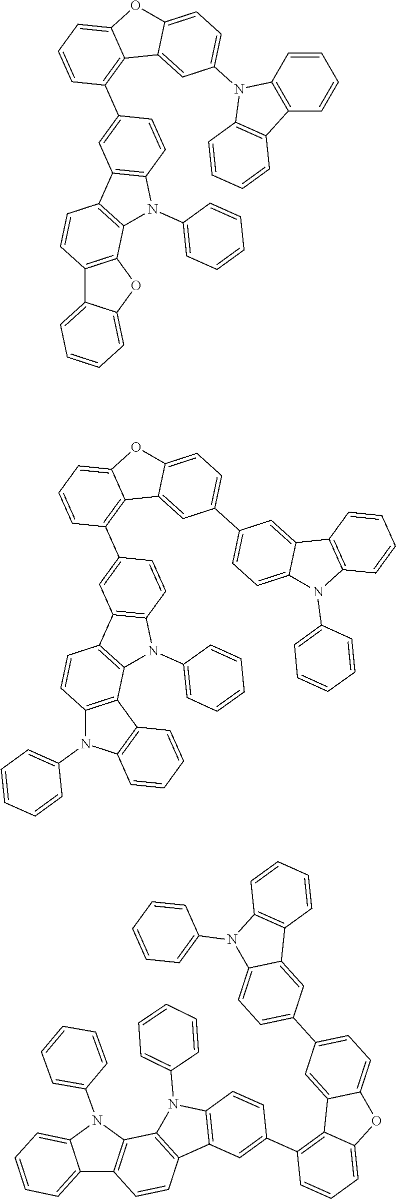 [SMILES: c1ccc(-n2c3ccc(-c4cccc5oc6ccc(-n7c8ccccc8c8ccccc87)cc6c45)cc3c3ccc4c5ccccc5oc4c32)cc1 ; c1ccc(-n2c3ccccc3c3cc(-c4ccc5oc6cccc(-c7ccc8c(c7)c7ccc9c(c%10ccccc%10n9-c9ccccc9)c7n8-c7ccccc7)c6c5c4)ccc32)cc1 ; c1ccc(-n2c3ccccc3c3cc(-c4ccc5oc6cccc(-c7ccc8c(c7)c7ccc9c%10ccccc%10n(-c%10ccccc%10)c9c7n8-c7ccccc7)c6c5c4)ccc32)cc1]

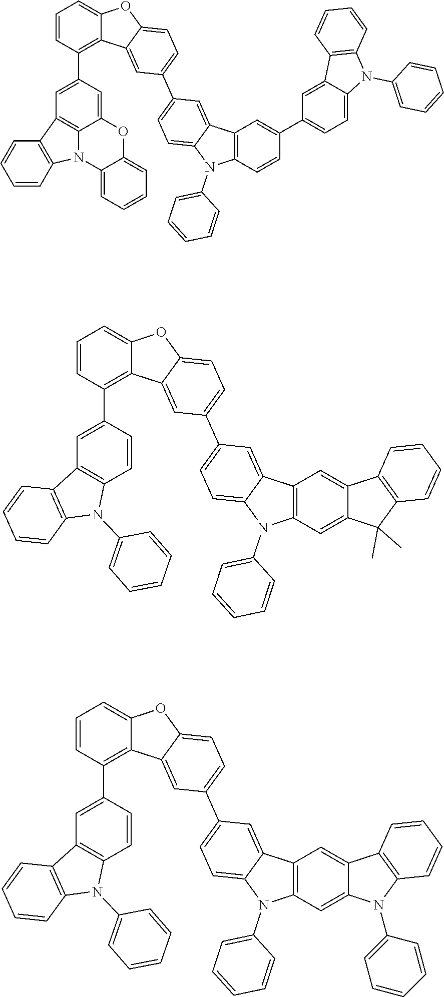 [SMILES: c1ccc(-n2c3ccccc3c3cc(-c4ccc5c(c4)c4cc(-c6ccc7oc8cccc(-c9cc%10c%11c(c9)c9ccccc9n%11-c9ccccc9O%10)c8c7c6)ccc4n5-c4ccccc4)ccc32)cc1 ; CC1(C)c2ccccc2-c2cc3c4cc(-c5ccc6oc7cccc(-c8ccc9c(c8)c8ccccc8n9-c8ccccc8)c7c6c5)ccc4n(-c4ccccc4)c3cc21 ; c1ccc(-n2c3ccccc3c3cc(-c4cccc5oc6ccc(-c7ccc8c(c7)c7cc9c%10ccccc%10n(-c%10ccccc%10)c9cc7n8-c7ccccc7)cc6c45)ccc32)cc1]

Preferred carbazoleamines are the structures of the following formulae (31), (32) and (33):

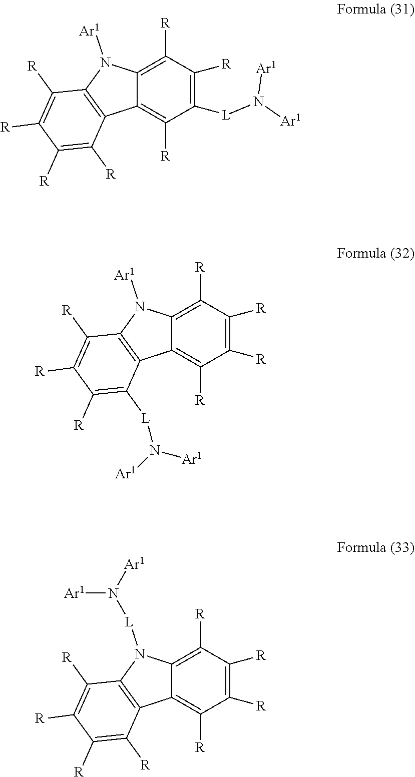

where L, R and Ar¹ have the definitions given above.

Examples of suitable carbazoleamine derivatives are the compounds depicted below.

[chemical structure]

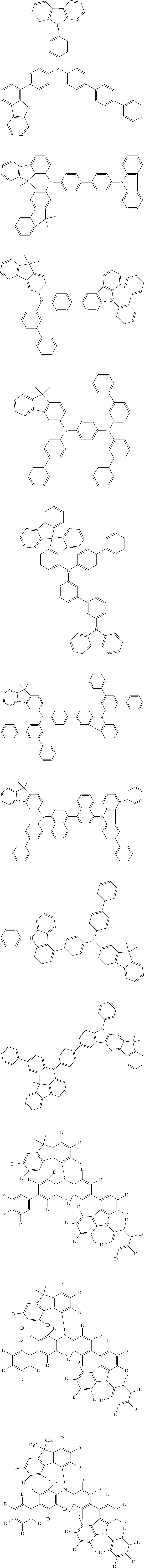

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, WO 2019/020538, WO 2018/178001, WO 2019/115423 and WO 2019/158453. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

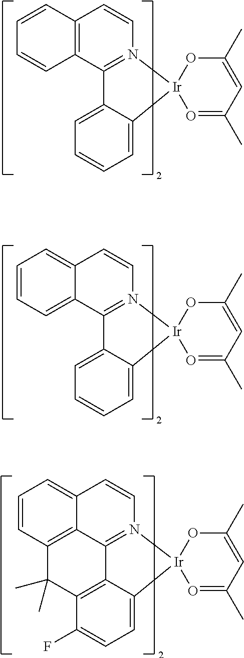

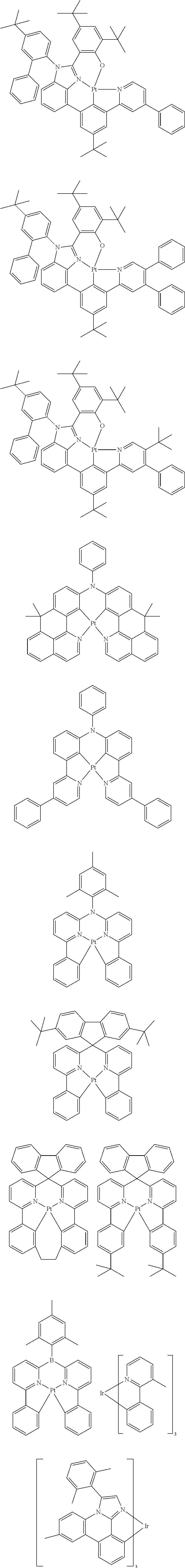

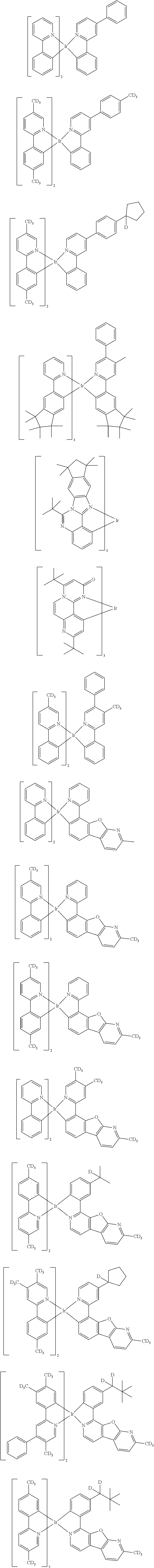

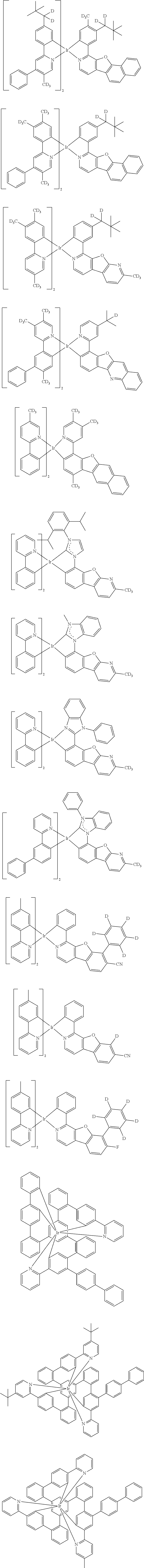

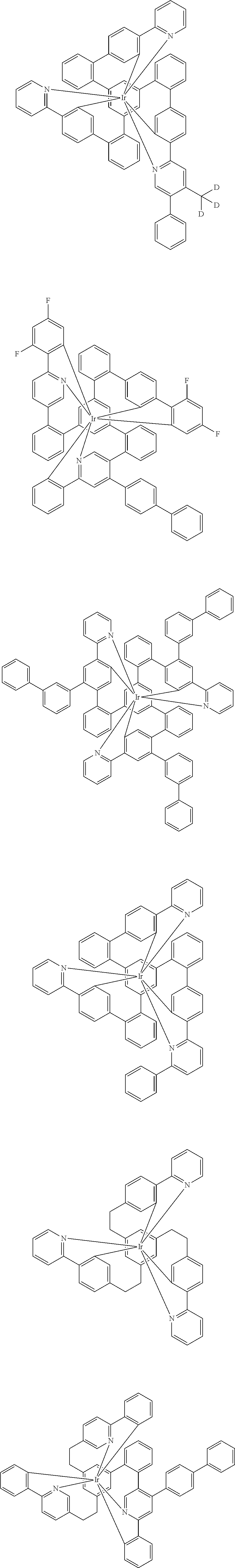

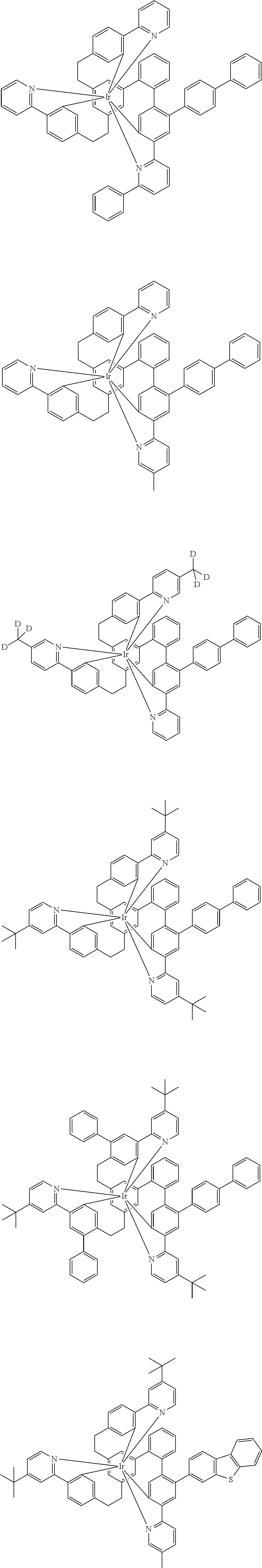

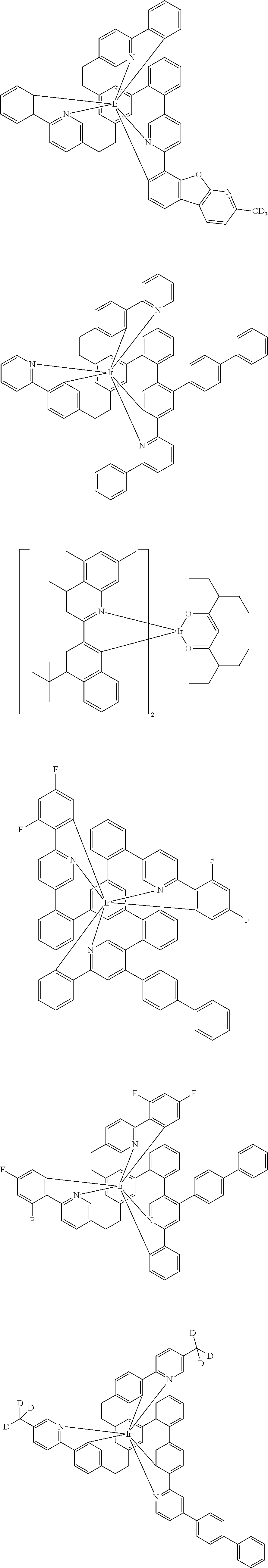

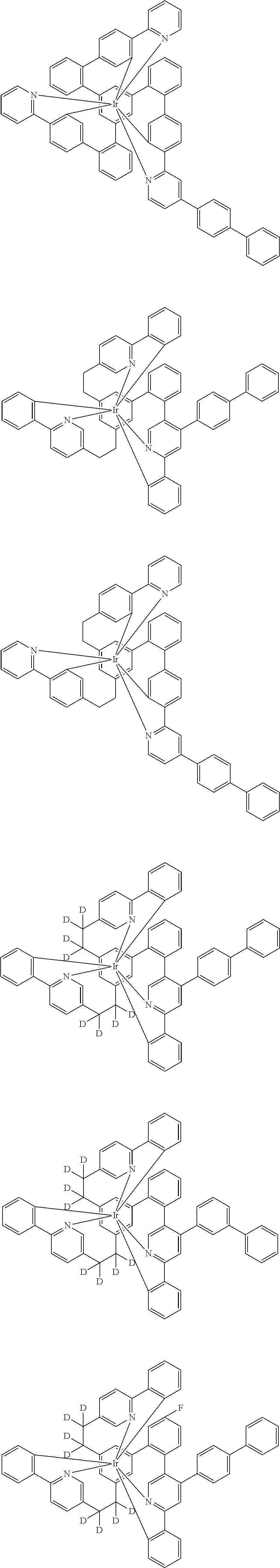

In the further layers of the organic electroluminescent device of the invention, it is possible to use any materials as typically used according to the prior art. The person skilled in the art will therefore be able, without exercising inventive skill, to use any materials known for organic electroluminescent devices in combination with the compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

These methods are known in general terms to those skilled in the art and can be applied by those skilled in the art without exercising inventive skill to organic electroluminescent devices comprising the compounds of formula (1).

The materials of the invention and the organic electroluminescent devices of the invention are notable for one or more of the following surprising advantages over the prior art:
1. OLEDs containing the compounds of formula (1) lead to high efficiencies. This is especially true when the compounds are used as matrix material for a phosphorescent emitter. In particular, the compounds show improved efficiency compared to OLEDs having matrix materials that have a dibenzothiophene in the base skeleton rather than a dibenzothiophene dioxide.
2. OLEDs containing the compounds of formula (1) lead to low operating voltages. This is especially true when the compounds are used as matrix material for a phosphorescent emitter. In particular, the compounds show a lower operating voltage compared to OLEDs having matrix materials that have a dibenzothiophene in the base skeleton rather than a dibenzothiophene dioxide.
3. OLEDs containing the compounds of formula (1) as matrix material for phosphorescent emitters lead to long lifetimes. This is especially true when the compounds are used as matrix material for a phosphorescent emitter.
4. The compounds of the invention may also be used with very good properties in an electron transport layer, including in combination with a fluorescent emission layer, or in a hole blocker layer.

The invention is illustrated in detail by the examples which follow, without any intention of restricting it thereby. The person skilled in the art will be able to use the information given to execute the invention over the entire scope disclosed and produce further inventive electronic devices without exercising inventive skill.

### SYNTHESIS EXAMPLES

The syntheses which follow, unless stated otherwise, are conducted under a protective gas atmosphere in dried solvents. The compounds of the invention can be prepared by means of synthesis methods known to those skilled in the art.

### Example 1

a) 1-Bromo-8-iododibenzothiophene 5,5-dioxide

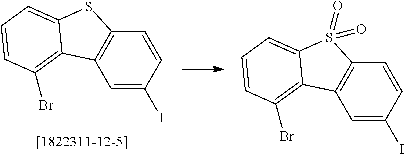 [SMILES: Brc1cccc2sc3ccc(I)cc3c12 ; O=S1(=O)c2ccc(I)cc2-c2c(Br)cccc21]

An initial charge under protective gas is formed by 18.9 g (49 mmol) of 1-bromo-8-iododibenzothiophene in 0.3 l of glacial acetic acid. To this solution are added dropwise 33 ml (618 mmol) of 30% H₂O₂ solution, and the mixture is stirred overnight. Na₂SO₃ solution is added to the mixture, the organic phase is separated off, and the solvent is removed under reduced pressure. The yield is 18.2 g (34 mmol), corresponding to 89% of theory.

The following compounds are prepared in an analogous manner:

| | Reactant 1 | Product | Yield |
|---|---|---|---|
| 1a | 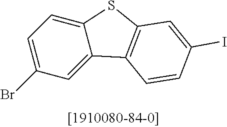 [SMILES: Brc1ccc2sc3cc(I)ccc3c2c1] | 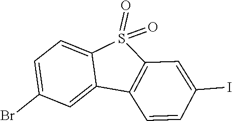 [SMILES: O=S1(=O)c2ccc(Br)cc2-c2ccc(I)cc21] | 58% |
| 2a | 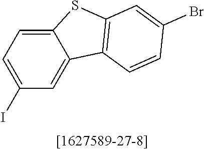 [SMILES: Brc1ccc2c(c1)sc1ccc(I)cc12] | 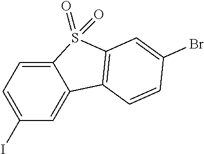 [SMILES: O=S1(=O)c2ccc(I)cc2-c2ccc(Br)cc21] | 61% |
| 3a | 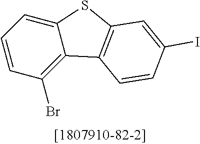 [SMILES: Brc1cccc2sc3cc(I)ccc3c12] | 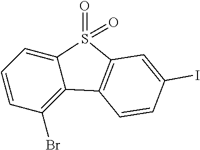 [SMILES: O=S1(=O)c2cc(I)ccc2-c2c(Br)cccc21] | 57% |

b) 3-Bromo-7-dibenzofuran-1-yldibenzothiophene 5,5-dioxide

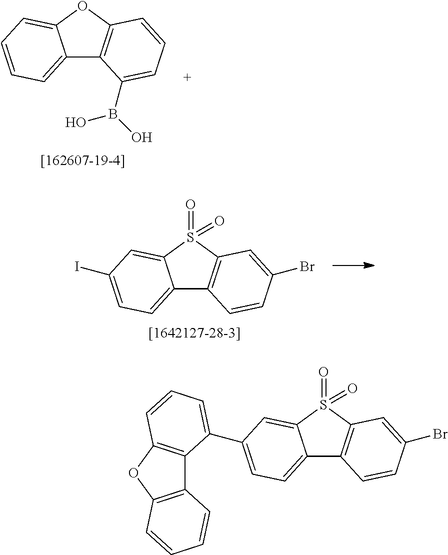 [SMILES: OB(O)c1cccc2oc3ccccc3c12 ; O=S1(=O)c2cc(Br)ccc2-c2ccc(I)cc21 ; O=S1(=O)c2cc(Br)ccc2-c2ccc(-c3cccc4oc5ccccc5c34)cc21]

23 g (110.0 mmol) of dibenzofuran-1-boronic acid, 46.3 g (110.0 mmol) of 3-bromo-7-iododibenzothiophene 5,5-dioxide and 21 g (210.0 mmol) of sodium carbonate are suspended in 500 ml of ethylene glycol dimethyl ether and 500 ml of water. To this suspension are added 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml of water and then concentrated to dryness. The residue is recrystallized from toluene and from dichloromethane/heptane. The yield is 37.5 g (81 mmol), corresponding to 75% of theory.

The following compounds are prepared in an analogous manner:

| | Reactant 1 | Reactant 2 | Product | Yield |
|---|---|---|---|---|
| 1b | 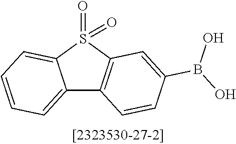 [SMILES: O=S1(=O)c2ccccc2-c2ccc(B(O)O)cc21] | 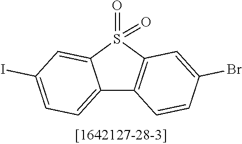 [SMILES: O=S1(=O)c2cc(Br)ccc2-c2ccc(I)cc21] | 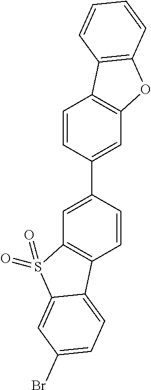 [SMILES: O=S1(=O)c2cc(Br)ccc2-c2ccc(-c3ccc4c(c3)oc3ccccc34)cc21] | 62% |
| 2b | 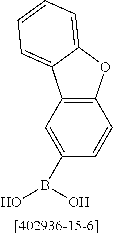 [SMILES: OB(O)c1ccc2oc3ccccc3c2c1] | 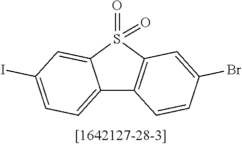 [SMILES: O=S1(=O)c2cc(Br)ccc2-c2ccc(I)cc21] | 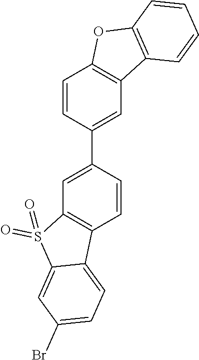 [SMILES: O=S1(=O)c2cc(Br)ccc2-c2ccc(-c3ccc4oc5ccccc5c4c3)cc21] | 61% |
| 3b | 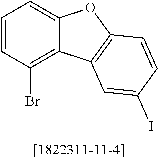 [SMILES: Brc1cccc2oc3ccc(I)cc3c12] | 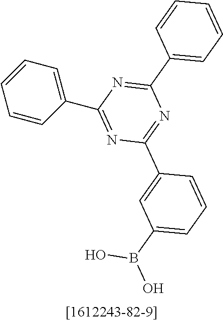 [SMILES: OB(O)c1cccc(-c2nc(-c3ccccc3)nc(-c3ccccc3)n2)c1] | 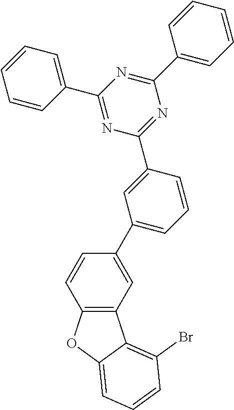 [SMILES: Brc1cccc2oc3ccc(-c4cccc(-c5nc(-c6ccccc6)nc(-c6ccccc6)n5)c4)cc3c12] | 65% |
| 4b | 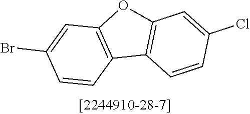 [SMILES: Clc1ccc2c(c1)oc1cc(Br)ccc12] | 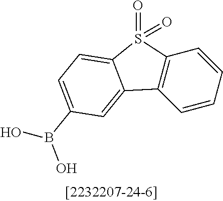 [SMILES: O=S1(=O)c2ccccc2-c2cc(B(O)O)ccc21] | 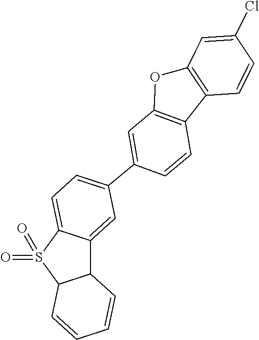 [SMILES: O=S1(=O)c2ccc(-c3ccc4c(c3)oc3cc(Cl)ccc34)cc2C2C=CC=CC21] | 61% |
| 5b | 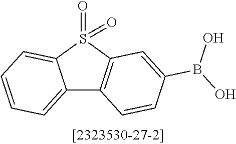 [SMILES: O=S1(=O)c2ccccc2-c2ccc(B(O)O)cc21] | 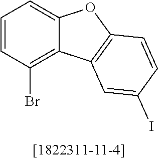 [SMILES: Brc1cccc2oc3ccc(I)cc3c12] | 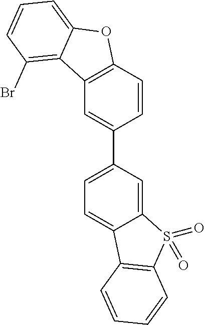 [SMILES: O=S1(=O)c2ccccc2-c2ccc(-c3ccc4oc5cccc(Br)c5c4c3)cc21] | 63% |
| 6b | 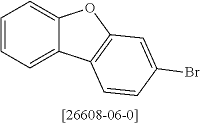 [SMILES: Brc1ccc2c(c1)oc1ccccc12] | 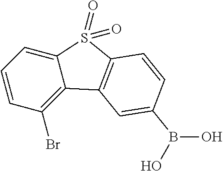 [SMILES: O=S1(=O)c2ccc(B(O)O)cc2-c2c(Br)cccc21] | 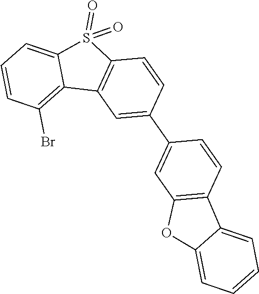 [SMILES: O=S1(=O)c2ccc(-c3ccc4c(c3)oc3ccccc34)cc2-c2c(Br)cccc21] | 60% |
| 7b | 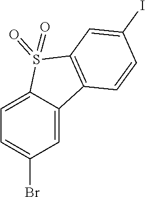 [SMILES: O=S1(=O)c2ccc(Br)cc2-c2ccc(I)cc21] | 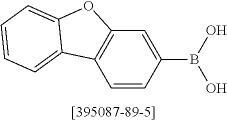 [SMILES: OB(O)c1ccc2c(c1)oc1ccccc12] | 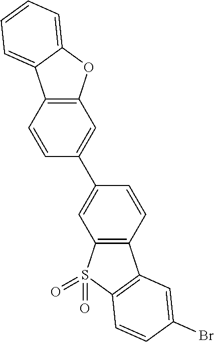 [SMILES: O=S1(=O)c2ccc(Br)cc2-c2ccc(-c3ccc4c(c3)oc3ccccc34)cc21] | 55% |
| 8b | 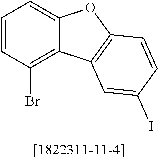 [SMILES: Brc1cccc2oc3ccc(I)cc3c12] | 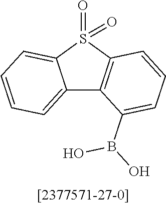 [SMILES: O=S1(=O)c2ccccc2-c2c(B(O)O)cccc21] | 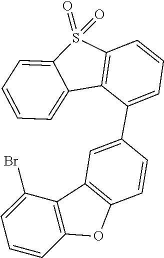 [SMILES: O=S1(=O)c2ccccc2-c2c(-c3ccc4oc5cccc(Br)c5c4c3)cccc21] | 63% |
| 9b | 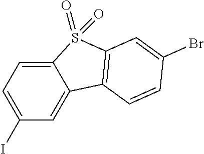 [SMILES: O=S1(=O)c2ccc(I)cc2-c2ccc(Br)cc21] | 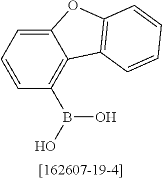 [SMILES: OB(O)c1cccc2oc3ccccc3c12] | 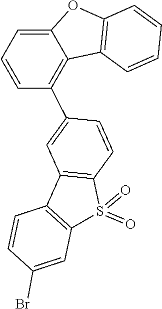 [SMILES: O=S1(=O)c2ccc(-c3cccc4oc5ccccc5c34)cc2-c2ccc(Br)cc21] | 69% |
| 10b | 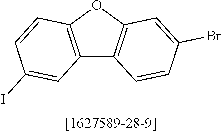 [SMILES: Brc1ccc2c(c1)oc1ccc(I)cc12] | 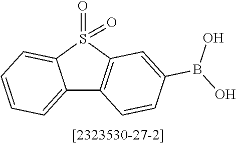 [SMILES: O=S1(=O)c2ccccc2-c2ccc(B(O)O)cc21] | 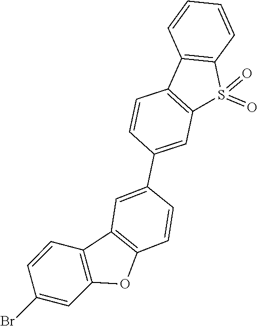 [SMILES: O=S1(=O)c2ccccc2-c2ccc(-c3ccc4oc5cc(Br)ccc5c4c3)cc21] | 56% |
| 11b | 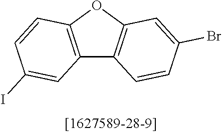 [SMILES: Brc1ccc2c(c1)oc1ccc(I)cc12] | 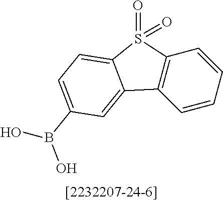 [SMILES: O=S1(=O)c2ccccc2-c2cc(B(O)O)ccc21] | 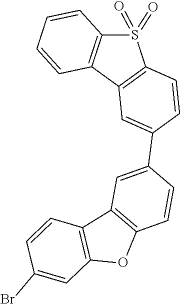 [SMILES: O=S1(=O)c2ccccc2-c2cc(-c3ccc4oc5cc(Br)ccc5c4c3)ccc21] | 64% |
| 12b | 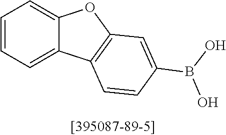 [SMILES: OB(O)c1ccc2c(c1)oc1ccccc12] | 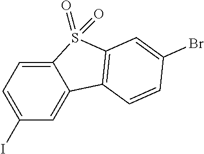 [SMILES: O=S1(=O)c2ccc(I)cc2-c2ccc(Br)cc21] | 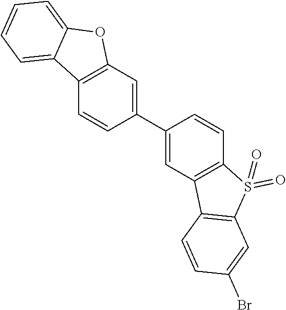 [SMILES: O=S1(=O)c2ccc(-c3ccc4c(c3)oc3ccccc34)cc2-c2ccc(Br)cc21] | 67% |
| 13b | 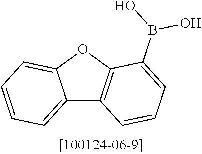 [SMILES: OB(O)c1cccc2c1oc1ccccc12] | 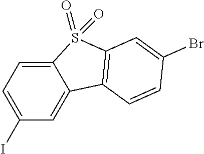 [SMILES: O=S1(=O)c2ccc(I)cc2-c2ccc(Br)cc21] | 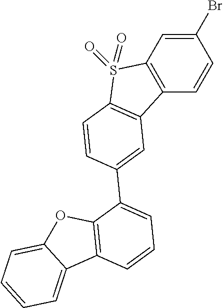 [SMILES: O=S1(=O)c2ccc(-c3cccc4c3oc3ccccc34)cc2-c2ccc(Br)cc21] | 73% |
| 14b | 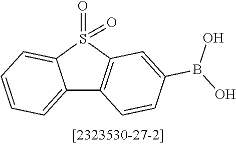 [SMILES: O=S1(=O)c2ccccc2-c2ccc(B(O)O)cc21] | 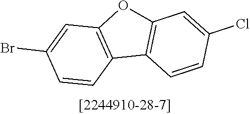 [SMILES: Clc1ccc2c(c1)oc1cc(Br)ccc12] | 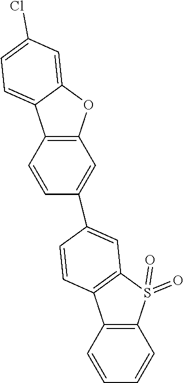 [SMILES: O=S1(=O)c2ccccc2-c2ccc(-c3ccc4c(c3)oc3cc(Cl)ccc34)cc21] | 78% |
| 15b | 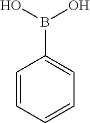 | 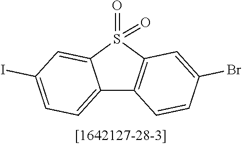 [SMILES: O=S1(=O)c2cc(Br)ccc2-c2ccc(I)cc21] | 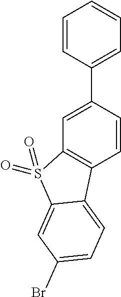 [SMILES: O=S1(=O)c2cc(Br)ccc2-c2ccc(-c3ccccc3)cc21] | 87% |
| 16b | 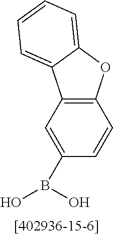 [SMILES: OB(O)c1ccc2oc3ccccc3c2c1] | 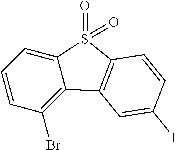 [SMILES: O=S1(=O)c2ccc(I)cc2-c2c(Br)cccc21] | 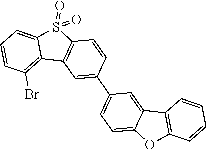 [SMILES: O=S1(=O)c2ccc(-c3ccc4oc5ccccc5c4c3)cc2-c2c(Br)cccc21] | 77% |
| 17b | 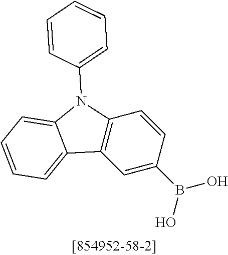 [SMILES: OB(O)c1ccc2c(c1)c1ccccc1n2-c1ccccc1] | 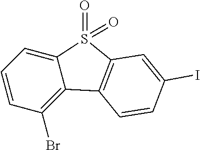 [SMILES: O=S1(=O)c2cc(I)ccc2-c2c(Br)cccc21] | 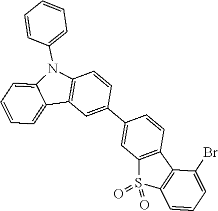 [SMILES: O=S1(=O)c2cc(-c3ccc4c(c3)c3ccccc3n4-c3ccccc3)ccc2-c2c(Br)cccc21] | 72% |
| 18b | 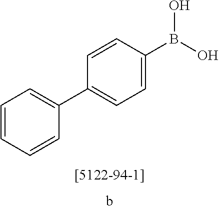 [SMILES: OB(O)c1ccc(-c2ccccc2)cc1] | 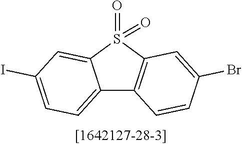 [SMILES: O=S1(=O)c2cc(Br)ccc2-c2ccc(I)cc21] | 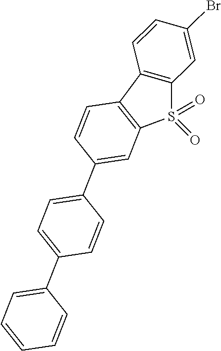 [SMILES: O=S1(=O)c2cc(Br)ccc2-c2ccc(-c3ccc(-c4ccccc4)cc3)cc21] | 61% |

c) 2-[4-(4,4,5,5-Tetramethyl-1,3,2-dioxaborolan-2-yl)phenyl]dibenzothiophene 5,5-dioxide

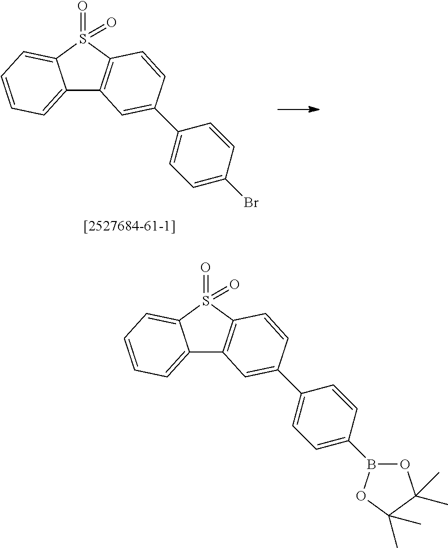 [SMILES: O=S1(=O)c2ccccc2-c2cc(-c3ccc(Br)cc3)ccc21 ; CC1(C)OB(c2ccc(-c3ccc4c(c3)-c3ccccc3S4(=O)=O)cc2)OC1(C)C]

An initial charge of 65 g (176 mmol) of 2-(4-bromophenyl)dibenzothiophene 5,5-dioxide, 82.1 g (320 mmol) of 4,4,5,5,4′,4′,5′,5′-octamethyl-[2,2′]bi[[1,3,2]dioxaborolanyl], 52.4 g (530 mmol) of potassium acetate and 2.8 g (12 mmol) of palladium acetate in 900 ml of N,N-dimethylformamide is stirred at 100° C. After 24 hours, the reaction mixture is allowed to cool down to room temperature, and the volume of the mixture is reduced to one third under reduced pressure, water is added, and the precipitated solids are filtered off, washed with water, ethanol and heptane, and purified further by filtration using a silica gel-packed column (THF as eluent). Subsequently, the solvent is removed on a rotary evaporator.

The yield is 44.6 g (106 mmol), corresponding to 61% of theory.

The following compounds are prepared in an analogous manner:

| | Reactant 1 | Product | Yield |
|---|---|---|---|
| 1c | 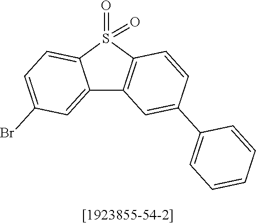 [SMILES: O=S1(=O)c2ccc(Br)cc2-c2cc(-c3ccccc3)ccc21] | 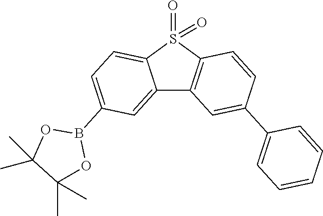 [SMILES: CC1(C)OB(c2ccc3c(c2)-c2cc(-c4ccccc4)ccc2S3(=O)=O)OC1(C)C] | 59% |
| 2c | 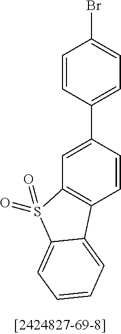 [SMILES: O=S1(=O)c2ccccc2-c2ccc(-c3ccc(Br)cc3)cc21] | 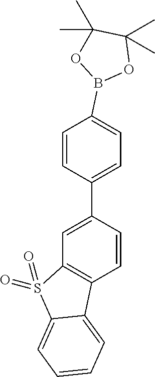 [SMILES: CC1(C)OB(c2ccc(-c3ccc4c(c3)S(=O)(=O)c3ccccc3-4)cc2)OC1(C)C] | 62% |
| 3c | 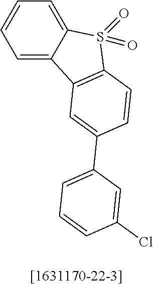 [SMILES: O=S1(=O)c2ccccc2-c2cc(-c3cccc(Cl)c3)ccc21] | 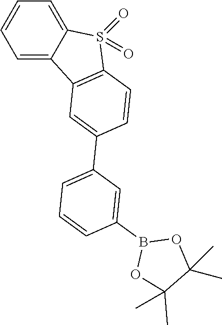 [SMILES: CC1(C)OB(c2cccc(-c3ccc4c(c3)-c3ccccc3S4(=O)=O)c2)OC1(C)C] | 58% |
| 4c | 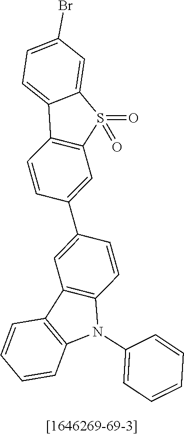 [SMILES: O=S1(=O)c2cc(Br)ccc2-c2ccc(-c3ccc4c(c3)c3ccccc3n4-c3ccccc3)cc21] | 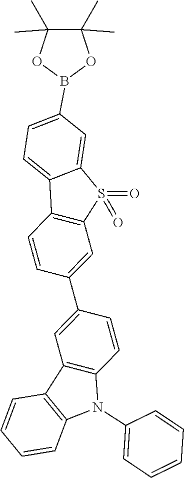 [SMILES: CC1(C)OB(c2ccc3c(c2)S(=O)(=O)c2cc(-c4ccc5c(c4)c4ccccc4n5-c4ccccc4)ccc2-3)OC1(C)C] | 65% |
| 5c | 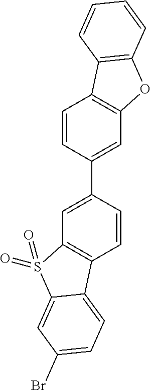 [SMILES: O=S1(=O)c2cc(Br)ccc2-c2ccc(-c3ccc4c(c3)oc3ccccc34)cc21] | 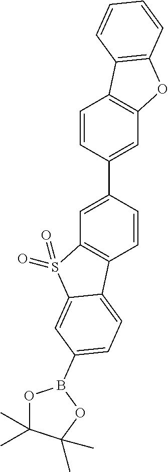 [SMILES: CC1(C)OB(c2ccc3c(c2)S(=O)(=O)c2cc(-c4ccc5c(c4)oc4ccccc45)ccc2-3)OC1(C)C] | 61% |
| 6c | 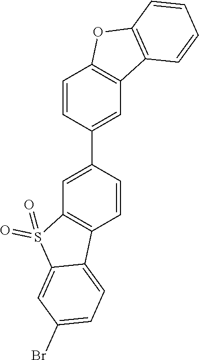 [SMILES: O=S1(=O)c2cc(Br)ccc2-c2ccc(-c3ccc4oc5ccccc5c4c3)cc21] | 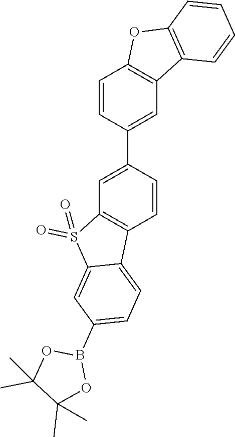 [SMILES: CC1(C)OB(c2ccc3c(c2)S(=O)(=O)c2cc(-c4ccc5oc6ccccc6c5c4)ccc2-3)OC1(C)C] | 54% |
| 7c | 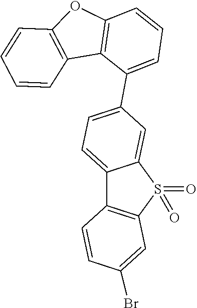 [SMILES: O=S1(=O)c2cc(Br)ccc2-c2ccc(-c3cccc4oc5ccccc5c34)cc21] | 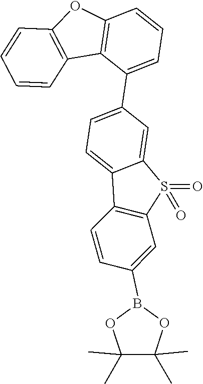 [SMILES: CC1(C)OB(c2ccc3c(c2)S(=O)(=O)c2cc(-c4cccc5oc6ccccc6c45)ccc2-3)OC1(C)C] | 63% |
| 8c | 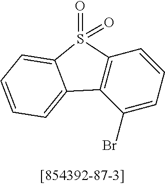 [SMILES: O=S1(=O)c2ccccc2-c2c(Br)cccc21] | 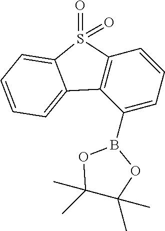 [SMILES: CC1(C)OB(c2cccc3c2-c2ccccc2S3(=O)=O)OC1(C)C] | 69% |
| 9c | 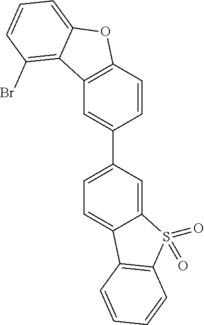 [SMILES: O=S1(=O)c2ccccc2-c2ccc(-c3ccc4oc5cccc(Br)c5c4c3)cc21] | 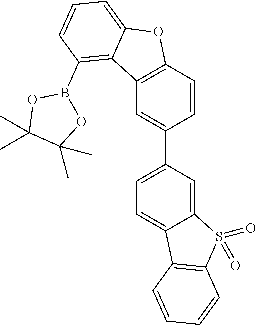 [SMILES: CC1(C)OB(c2cccc3oc4ccc(-c5ccc6c(c5)S(=O)(=O)c5ccccc5-6)cc4c23)OC1(C)C] | 58% |
| 10c | 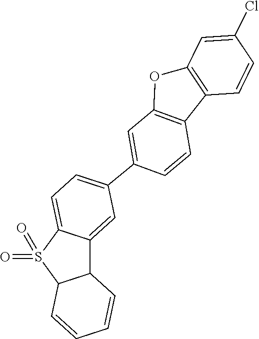 [SMILES: O=S1(=O)c2ccc(-c3ccc4c(c3)oc3cc(Cl)ccc34)cc2C2C=CC=CC21] | 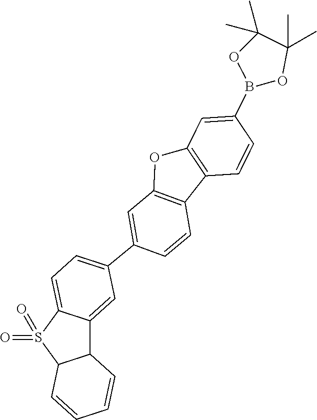 [SMILES: CC1(C)OB(c2ccc3c(c2)oc2cc(-c4ccc5c(c4)C4C=CC=CC4S5(=O)=O)ccc23)OC1(C)C] | 47% |
| 11c | 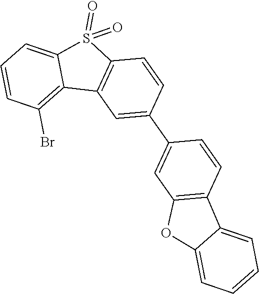 [SMILES: O=S1(=O)c2ccc(-c3ccc4c(c3)oc3ccccc34)cc2-c2c(Br)cccc21] | 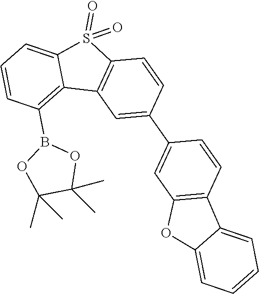 [SMILES: CC1(C)OB(c2cccc3c2-c2cc(-c4ccc5c(c4)oc4ccccc45)ccc2S3(=O)=O)OC1(C)C] | 43% |
| 12c | 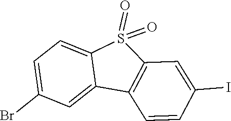 [SMILES: O=S1(=O)c2ccc(Br)cc2-c2ccc(I)cc21] | 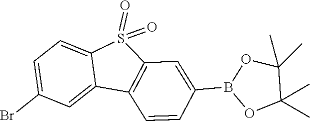 [SMILES: CC1(C)OB(c2ccc3c(c2)S(=O)(=O)c2ccc(Br)cc2-3)OC1(C)C] | 62% |
| 13c | 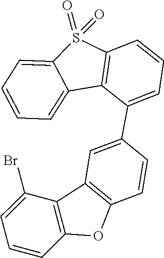 [SMILES: O=S1(=O)c2ccccc2-c2c(-c3ccc4oc5cccc(Br)c5c4c3)cccc21] | 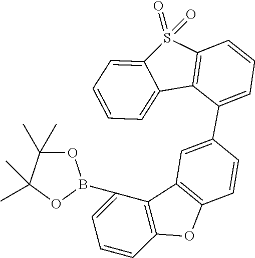 [SMILES: CC1(C)OB(c2cccc3oc4ccc(-c5cccc6c5-c5ccccc5S6(=O)=O)cc4c23)OC1(C)C] | 67% |
| 14c | 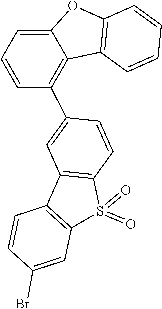 [SMILES: O=S1(=O)c2ccc(-c3cccc4oc5ccccc5c34)cc2-c2ccc(Br)cc21] | 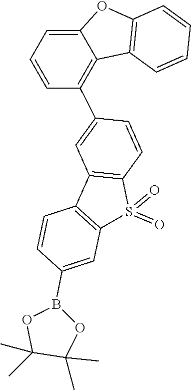 [SMILES: CC1(C)OB(c2ccc3c(c2)S(=O)(=O)c2ccc(-c4cccc5oc6ccccc6c45)cc2-3)OC1(C)C] | 58% |
| 15c | 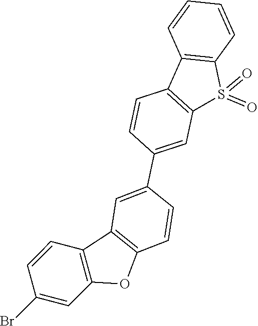 [SMILES: O=S1(=O)c2ccccc2-c2ccc(-c3ccc4oc5cc(Br)ccc5c4c3)cc21] | 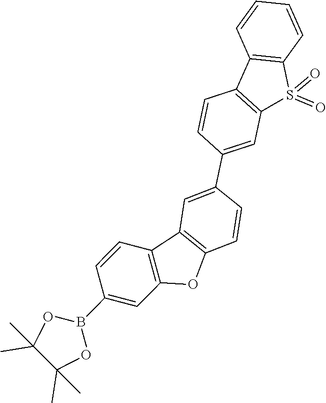 [SMILES: CC1(C)OB(c2ccc3c(c2)oc2ccc(-c4ccc5c(c4)S(=O)(=O)c4ccccc4-5)cc23)OC1(C)C] | 61% |
| 16c | 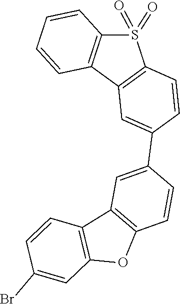 [SMILES: O=S1(=O)c2ccccc2-c2cc(-c3ccc4oc5cc(Br)ccc5c4c3)ccc21] | 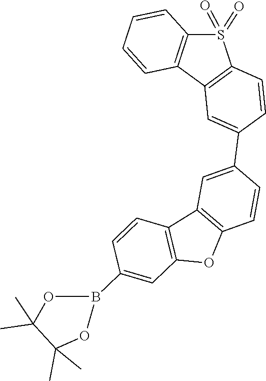 [SMILES: CC1(C)OB(c2ccc3c(c2)oc2ccc(-c4ccc5c(c4)-c4ccccc4S5(=O)=O)cc23)OC1(C)C] | 66% |
| 17c | 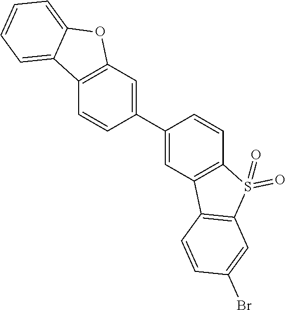 [SMILES: O=S1(=O)c2ccc(-c3ccc4c(c3)oc3ccccc34)cc2-c2ccc(Br)cc21] | 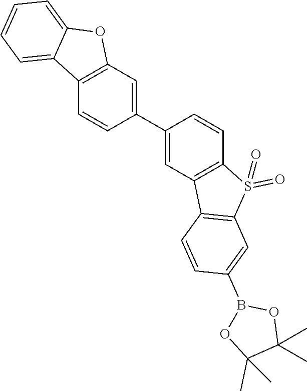 [SMILES: CC1(C)OB(c2ccc3c(c2)S(=O)(=O)c2ccc(-c4ccc5c(c4)oc4ccccc45)cc2-3)OC1(C)C] | 71% |
| 18c | 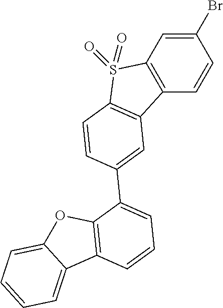 [SMILES: O=S1(=O)c2ccc(-c3cccc4c3oc3ccccc34)cc2-c2ccc(Br)cc21] | 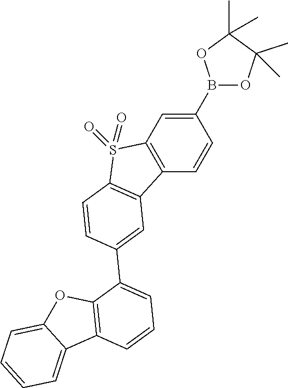 [SMILES: CC1(C)OB(c2ccc3c(c2)S(=O)(=O)c2ccc(-c4cccc5c4oc4ccccc45)cc2-3)OC1(C)C] | 68% |
| 19c | 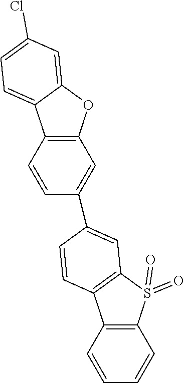 [SMILES: O=S1(=O)c2ccccc2-c2ccc(-c3ccc4c(c3)oc3cc(Cl)ccc34)cc21] | 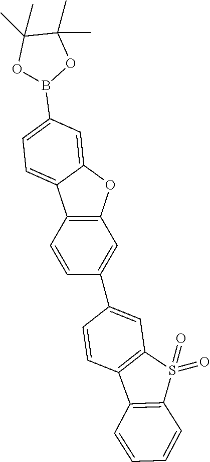 [SMILES: CC1(C)OB(c2ccc3c(c2)oc2cc(-c4ccc5c(c4)S(=O)(=O)c4ccccc4-5)ccc23)OC1(C)C] | 54% |
| 20c | 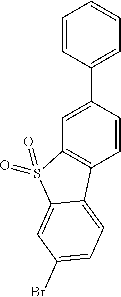 [SMILES: O=S1(=O)c2cc(Br)ccc2-c2ccc(-c3ccccc3)cc21] | 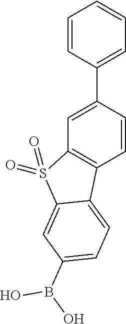 [SMILES: O=S1(=O)c2cc(B(O)O)ccc2-c2ccc(-c3ccccc3)cc21] | 66% |
| 21c | 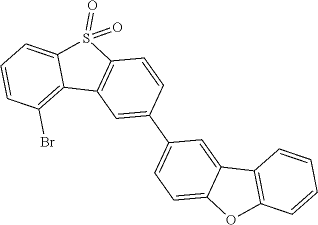 [SMILES: O=S1(=O)c2ccc(-c3ccc4oc5ccccc5c4c3)cc2-c2c(Br)cccc21] | 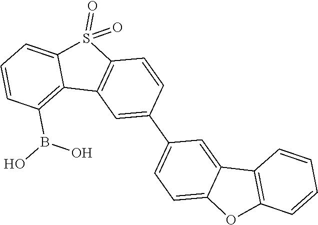 [SMILES: O=S1(=O)c2ccc(-c3ccc4oc5ccccc5c4c3)cc2-c2c(B(O)O)cccc21] | 63% |
| 22c | 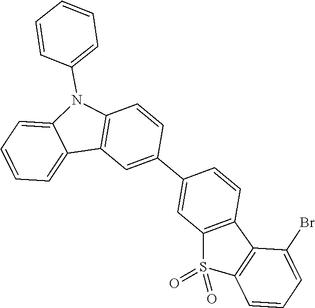 [SMILES: O=S1(=O)c2cc(-c3ccc4c(c3)c3ccccc3n4-c3ccccc3)ccc2-c2c(Br)cccc21] | 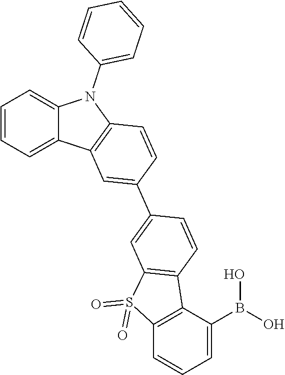 [SMILES: O=S1(=O)c2cc(-c3ccc4c(c3)c3ccccc3n4-c3ccccc3)ccc2-c2c(B(O)O)cccc21] | 58% |
| 23c | 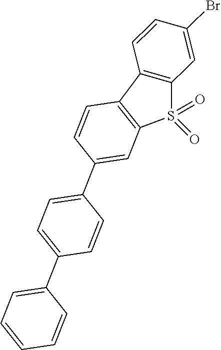 [SMILES: O=S1(=O)c2cc(Br)ccc2-c2ccc(-c3ccc(-c4ccccc4)cc3)cc21] | 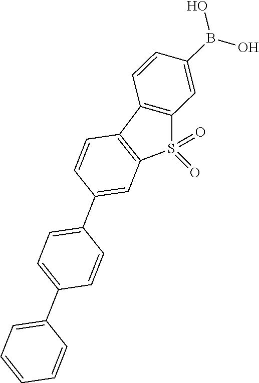 [SMILES: O=S1(=O)c2cc(B(O)O)ccc2-c2ccc(-c3ccc(-c4ccccc4)cc3)cc21] | 66% |

d) 2-[4-(4,6-Diphenyl-1,3,5-triazin-2-yl)phenyl]dibenzothiophene 5,5-dioxide

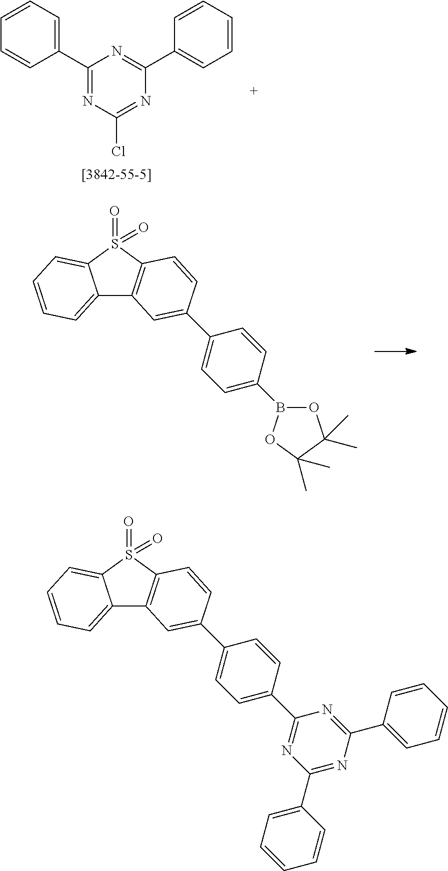 [SMILES: Clc1nc(-c2ccccc2)nc(-c2ccccc2)n1 ; CC1(C)OB(c2ccc(-c3ccc4c(c3)-c3ccccc3S4(=O)=O)cc2)OC1(C)C ; O=S1(=O)c2ccccc2-c2cc(-c3ccc(-c4nc(-c5ccccc5)nc(-c5ccccc5)n4)cc3)ccc21]

54.3 g (130 mmol) of 2-[4-(4,4,5,5-tetramethyl-1,3,2-dioxaborolan-2-yl)phenyl]dibenzothiophene 5,5-dioxide, 33 g (124.1 mmol) of 2-chloro-4,6-diphenyl-[1,3,5]triazine and 79 ml (158 mmol) of Na₂CO₃ (2 M solution) are suspended in 120 ml of toluene, 120 ml of ethanol and 100 ml of water. 2.6 g (2.2 mmol) of Pd(PPh₃)₄ is added to this suspension, and the reaction mixture is heated under reflux for 16 h. After cooling, the organic phase is removed, filtered through silica gel, washed three times with 200 ml of water and then concentrated to dryness. The residue is recrystallized from toluene. The yield is 56 g (107 mmol), corresponding to 87% of theory.

The compounds that follow can be obtained analogously.

| | Reactant 1 | Reactant 2 |
|---|---|---|
| 1d | 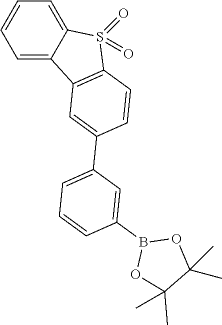 [SMILES: CC1(C)OB(c2cccc(-c3ccc4c(c3)-c3ccccc3S4(=O)=O)c2)OC1(C)C] | 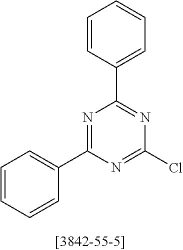 [SMILES: Clc1nc(-c2ccccc2)nc(-c2ccccc2)n1] |
| 2d | 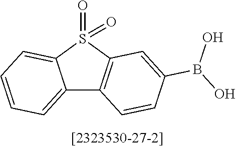 [SMILES: O=S1(=O)c2ccccc2-c2ccc(B(O)O)cc21] | 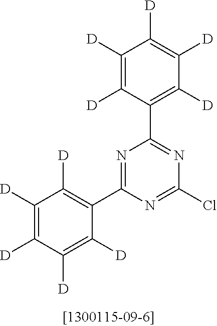 [SMILES: [2H]c1c([2H])c([2H])c(-c2nc(Cl)nc(-c3c([2H])c([2H])c([2H])c([2H])c3[2H])n2)c([2H])c1[2H]] |
| 3d | 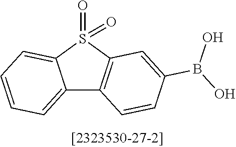 [SMILES: O=S1(=O)c2ccccc2-c2ccc(B(O)O)cc21] | 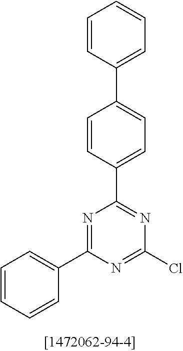 [SMILES: Clc1nc(-c2ccccc2)nc(-c2ccc(-c3ccccc3)cc2)n1] |
| 4d | 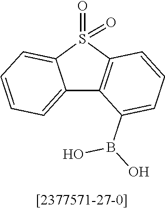 [SMILES: O=S1(=O)c2ccccc2-c2c(B(O)O)cccc21] | 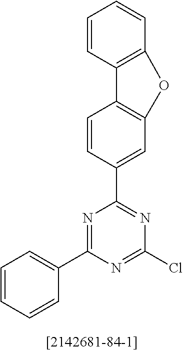 [SMILES: Clc1nc(-c2ccccc2)nc(-c2ccc3c(c2)oc2ccccc23)n1] |
| 5d | 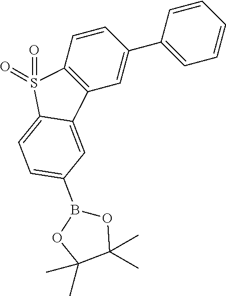 [SMILES: CC1(C)OB(c2ccc3c(c2)-c2cc(-c4ccccc4)ccc2S3(=O)=O)OC1(C)C] | 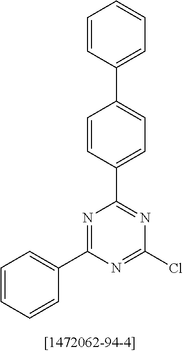 [SMILES: Clc1nc(-c2ccccc2)nc(-c2ccc(-c3ccccc3)cc2)n1] |
| 6d | 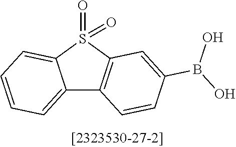 [SMILES: O=S1(=O)c2ccccc2-c2ccc(B(O)O)cc21] | 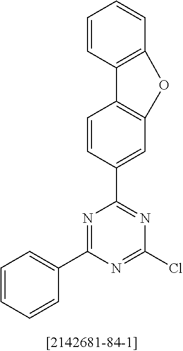 [SMILES: Clc1nc(-c2ccccc2)nc(-c2ccc3c(c2)oc2ccccc23)n1] |
| 7d | 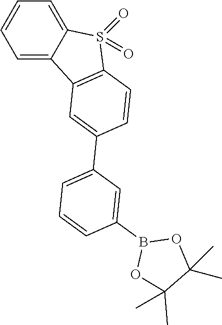 [SMILES: CC1(C)OB(c2cccc(-c3ccc4c(c3)-c3ccccc3S4(=O)=O)c2)OC1(C)C] | 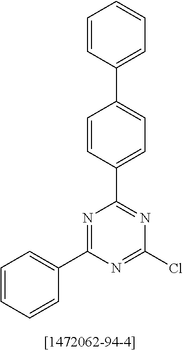 [SMILES: Clc1nc(-c2ccccc2)nc(-c2ccc(-c3ccccc3)cc2)n1] |
| 8d | 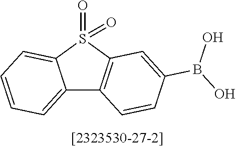 [SMILES: O=S1(=O)c2ccccc2-c2ccc(B(O)O)cc21] | 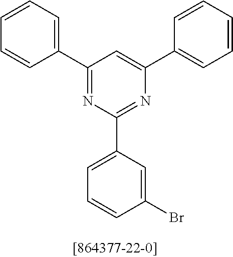 [SMILES: Brc1cccc(-c2nc(-c3ccccc3)cc(-c3ccccc3)n2)c1] |
| 9d | 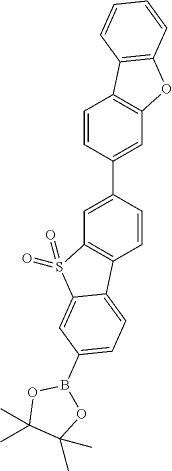 [SMILES: CC1(C)OB(c2ccc3c(c2)S(=O)(=O)c2cc(-c4ccc5c(c4)oc4ccccc45)ccc2-3)OC1(C)C] | 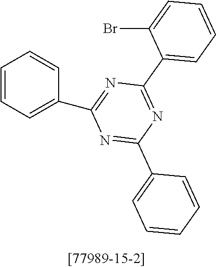 [SMILES: Brc1ccccc1-c1nc(-c2ccccc2)nc(-c2ccccc2)n1] |
| 10d | 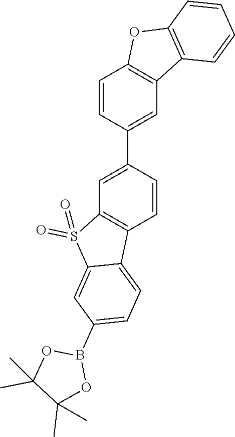 [SMILES: CC1(C)OB(c2ccc3c(c2)S(=O)(=O)c2cc(-c4ccc5oc6ccccc6c5c4)ccc2-3)OC1(C)C] | 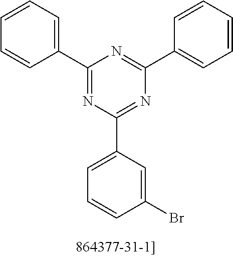 [SMILES: Brc1cccc(-c2nc(-c3ccccc3)nc(-c3ccccc3)n2)c1] |
| 11d | 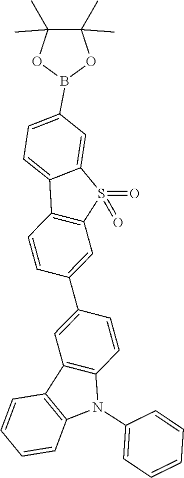 [SMILES: CC1(C)OB(c2ccc3c(c2)S(=O)(=O)c2cc(-c4ccc5c(c4)c4ccccc4n5-c4ccccc4)ccc2-3)OC1(C)C] | 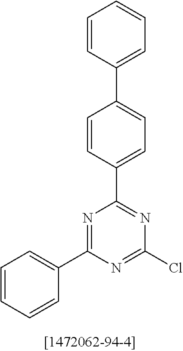 [SMILES: Clc1nc(-c2ccccc2)nc(-c2ccc(-c3ccccc3)cc2)n1] |
| 12d | 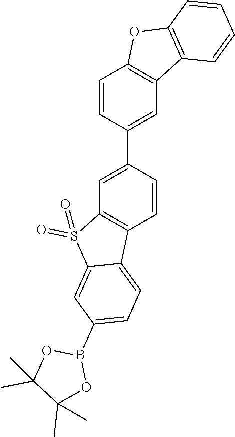 [SMILES: CC1(C)OB(c2ccc3c(c2)S(=O)(=O)c2cc(-c4ccc5oc6ccccc6c5c4)ccc2-3)OC1(C)C] | 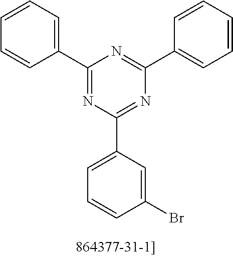 [SMILES: Brc1cccc(-c2nc(-c3ccccc3)nc(-c3ccccc3)n2)c1] |
| 13d | 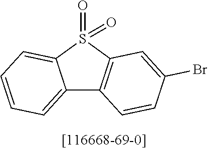 [SMILES: O=S1(=O)c2ccccc2-c2ccc(Br)cc21] | 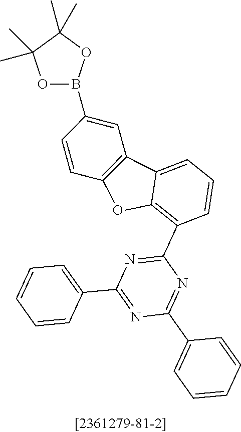 [SMILES: CC1(C)OB(c2ccc3oc4c(-c5nc(-c6ccccc6)nc(-c6ccccc6)n5)cccc4c3c2)OC1(C)C] |
| 14d | 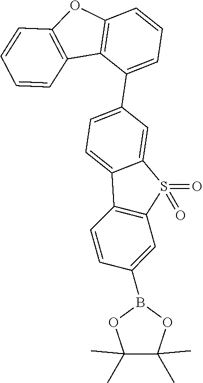 [SMILES: CC1(C)OB(c2ccc3c(c2)S(=O)(=O)c2cc(-c4cccc5oc6ccccc6c45)ccc2-3)OC1(C)C] | 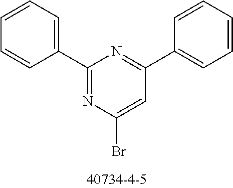 [SMILES: Brc1cc(-c2ccccc2)nc(-c2ccccc2)n1] |
| 15d | 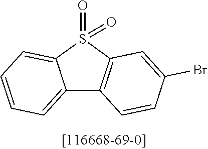 [SMILES: O=S1(=O)c2ccccc2-c2ccc(Br)cc21] | 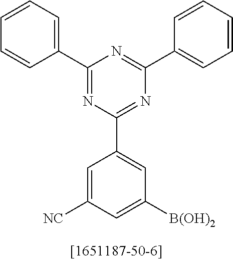 [SMILES: N#Cc1cc(B(O)O)cc(-c2nc(-c3ccccc3)nc(-c3ccccc3)n2)c1] |
| 16d | 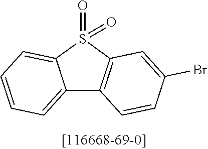 [SMILES: O=S1(=O)c2ccccc2-c2ccc(Br)cc21] | 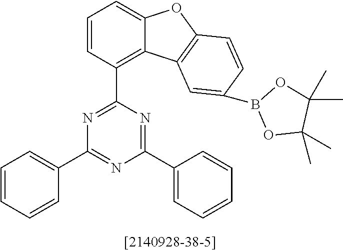 [SMILES: CC1(C)OB(c2ccc3oc4cccc(-c5nc(-c6ccccc6)nc(-c6ccccc6)n5)c4c3c2)OC1(C)C] |
| 17d | 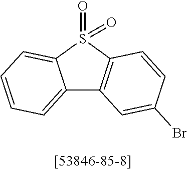 [SMILES: O=S1(=O)c2ccccc2-c2cc(Br)ccc21] | 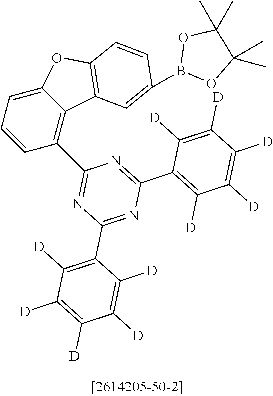 [SMILES: [2H]c1c([2H])c([2H])c(-c2nc(-c3c([2H])c([2H])c([2H])c([2H])c3[2H])nc(-c3cccc4oc5ccc(B6OC(C)(C)C(C)(C)O6)cc5c34)n2)c([2H])c1[2H]] |
| 18d | 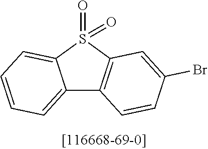 [SMILES: O=S1(=O)c2ccccc2-c2ccc(Br)cc21] | 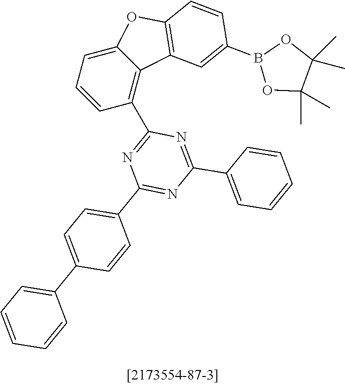 [SMILES: CC1(C)OB(c2ccc3oc4cccc(-c5nc(-c6ccccc6)nc(-c6ccc(-c7ccccc7)cc6)n5)c4c3c2)OC1(C)C] |
| 19d | 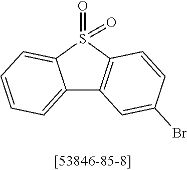 [SMILES: O=S1(=O)c2ccccc2-c2cc(Br)ccc21] | 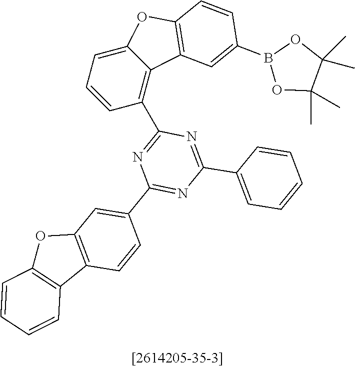 [SMILES: CC1(C)OB(c2ccc3oc4cccc(-c5nc(-c6ccccc6)nc(-c6ccc7c(c6)oc6ccccc67)n5)c4c3c2)OC1(C)C] |
| 20d | 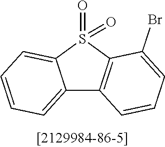 [SMILES: O=S1(=O)c2ccccc2-c2cccc(Br)c21] | 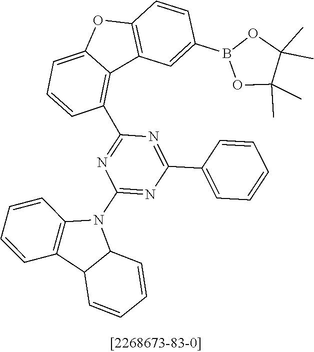 [SMILES: CC1(C)OB(c2ccc3oc4cccc(-c5nc(-c6ccccc6)nc(N6c7ccccc7C7C=CC=CC76)n5)c4c3c2)OC1(C)C] |
| 21d | 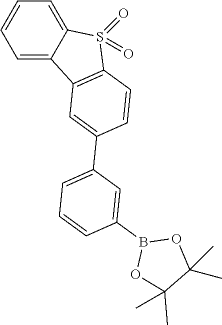 [SMILES: CC1(C)OB(c2cccc(-c3ccc4c(c3)-c3ccccc3S4(=O)=O)c2)OC1(C)C] | 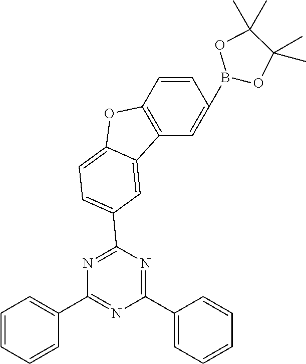 [SMILES: CC1(C)OB(c2ccc3oc4ccc(-c5nc(-c6ccccc6)nc(-c6ccccc6)n5)cc4c3c2)OC1(C)C] |
| 22d | 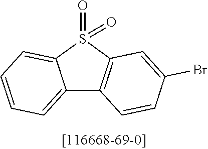 [SMILES: O=S1(=O)c2ccccc2-c2ccc(Br)cc21] | 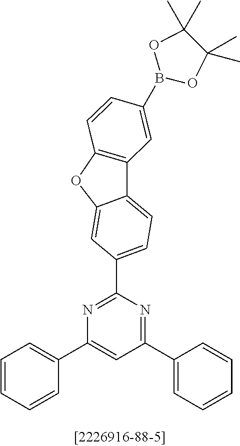 [SMILES: CC1(C)OB(c2ccc3oc4cc(-c5nc(-c6ccccc6)cc(-c6ccccc6)n5)ccc4c3c2)OC1(C)C] |
| 23d | 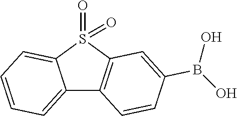 [SMILES: O=S1(=O)c2ccccc2-c2ccc(B(O)O)cc21] | 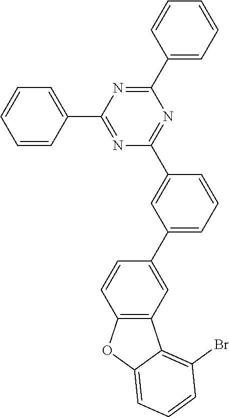 [SMILES: Brc1cccc2oc3ccc(-c4cccc(-c5nc(-c6ccccc6)nc(-c6ccccc6)n5)c4)cc3c12] |
| 24d | 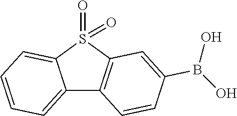 [SMILES: O=S1(=O)c2ccccc2-c2ccc(B(O)O)cc21] | 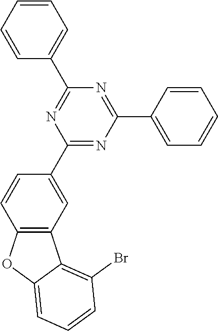 [SMILES: Brc1cccc2oc3ccc(-c4nc(-c5ccccc5)nc(-c5ccccc5)n4)cc3c12] |
| 25d | 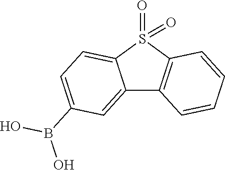 [SMILES: O=S1(=O)c2ccccc2-c2cc(B(O)O)ccc21] | 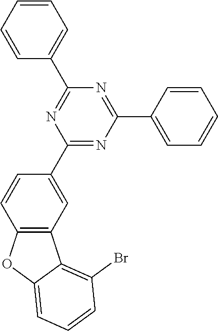 [SMILES: Brc1cccc2oc3ccc(-c4nc(-c5ccccc5)nc(-c5ccccc5)n4)cc3c12] |
| 25d | 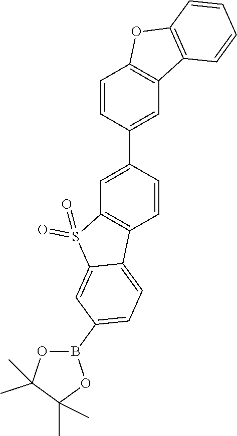 [SMILES: CC1(C)OB(c2ccc3c(c2)S(=O)(=O)c2cc(-c4ccc5oc6ccccc6c5c4)ccc2-3)OC1(C)C] | 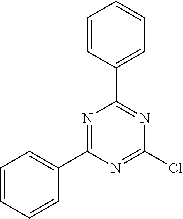 [SMILES: Clc1nc(-c2ccccc2)nc(-c2ccccc2)n1] |
| 26d | 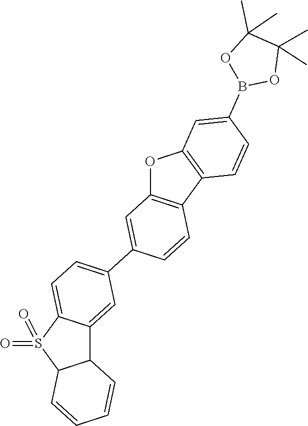 [SMILES: CC1(C)OB(c2ccc3c(c2)oc2cc(-c4ccc5c(c4)C4C=CC=CC4S5(=O)=O)ccc23)OC1(C)C] | 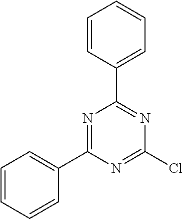 [SMILES: Clc1nc(-c2ccccc2)nc(-c2ccccc2)n1] |
| 27d | 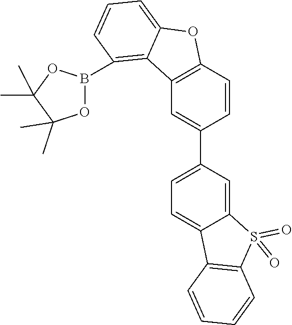 [SMILES: CC1(C)OB(c2cccc3oc4ccc(-c5ccc6c(c5)S(=O)(=O)c5ccccc5-6)cc4c23)OC1(C)C] | 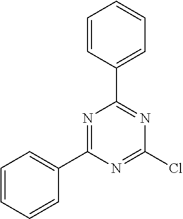 [SMILES: Clc1nc(-c2ccccc2)nc(-c2ccccc2)n1] |
| 28d | 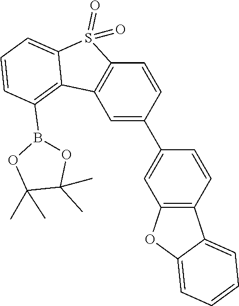 [SMILES: CC1(C)OB(c2cccc3c2-c2cc(-c4ccc5c(c4)oc4ccccc45)ccc2S3(=O)=O)OC1(C)C] | 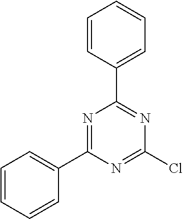 [SMILES: Clc1nc(-c2ccccc2)nc(-c2ccccc2)n1] |
| 29d | 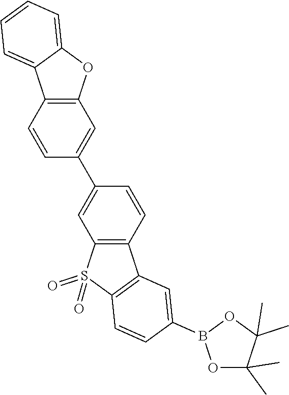 [SMILES: CC1(C)OB(c2ccc3c(c2)-c2ccc(-c4ccc5c(c4)oc4ccccc45)cc2S3(=O)=O)OC1(C)C] | 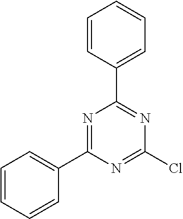 [SMILES: Clc1nc(-c2ccccc2)nc(-c2ccccc2)n1] |
| 30d | 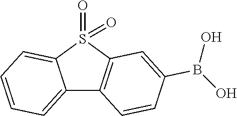 [SMILES: O=S1(=O)c2ccccc2-c2ccc(B(O)O)cc21] | 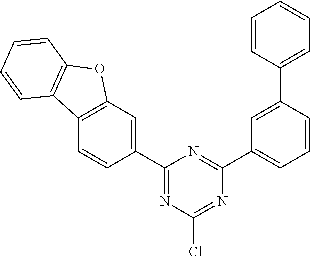 [SMILES: Clc1nc(-c2cccc(-c3ccccc3)c2)nc(-c2ccc3c(c2)oc2ccccc23)n1] |
| 31d | 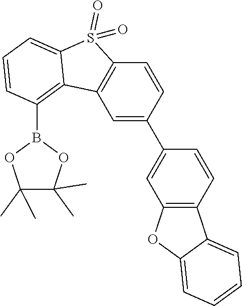 [SMILES: CC1(C)OB(c2cccc3c2-c2cc(-c4ccc5c(c4)oc4ccccc45)ccc2S3(=O)=O)OC1(C)C] | 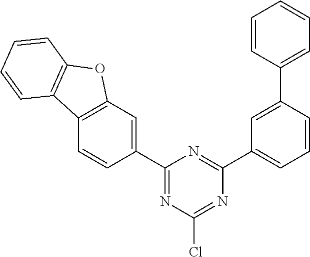 [SMILES: Clc1nc(-c2cccc(-c3ccccc3)c2)nc(-c2ccc3c(c2)oc2ccccc23)n1] |
| 32d | 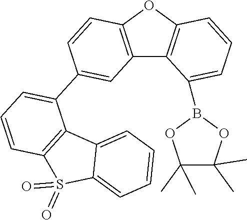 [SMILES: CC1(C)OB(c2cccc3oc4ccc(-c5cccc6c5-c5ccccc5S6(=O)=O)cc4c23)OC1(C)C] | 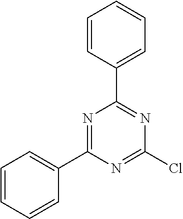 [SMILES: Clc1nc(-c2ccccc2)nc(-c2ccccc2)n1] |
| 33d | 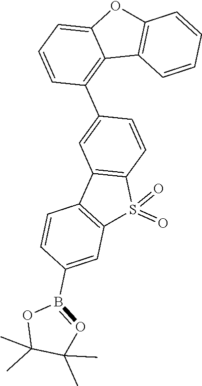 | 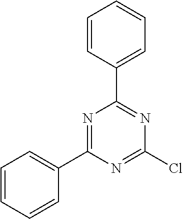 [SMILES: Clc1nc(-c2ccccc2)nc(-c2ccccc2)n1] |
| 34d | 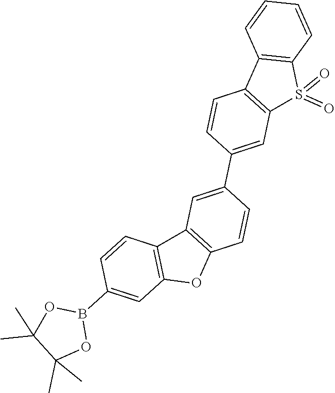 [SMILES: CC1(C)OB(c2ccc3c(c2)oc2ccc(-c4ccc5c(c4)S(=O)(=O)c4ccccc4-5)cc23)OC1(C)C] | 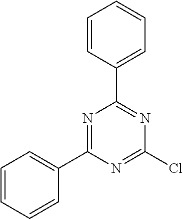 [SMILES: Clc1nc(-c2ccccc2)nc(-c2ccccc2)n1] |
| 35d | 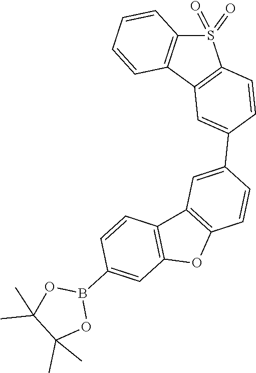 [SMILES: CC1(C)OB(c2ccc3c(c2)oc2ccc(-c4ccc5c(c4)-c4ccccc4S5(=O)=O)cc23)OC1(C)C] | 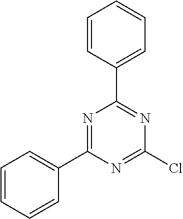 [SMILES: Clc1nc(-c2ccccc2)nc(-c2ccccc2)n1] |
| 36d | 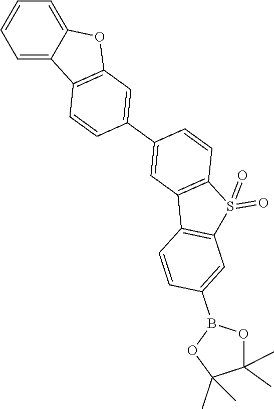 [SMILES: CC1(C)OB(c2ccc3c(c2)S(=O)(=O)c2ccc(-c4ccc5c(c4)oc4ccccc45)cc2-3)OC1(C)C] | 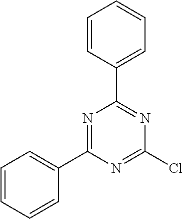 [SMILES: Clc1nc(-c2ccccc2)nc(-c2ccccc2)n1] |
| 37d | 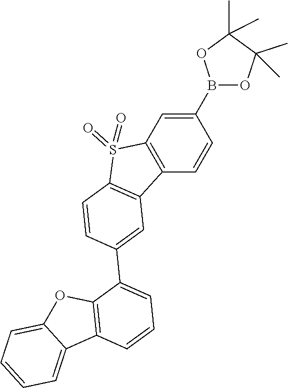 [SMILES: CC1(C)OB(c2ccc3c(c2)S(=O)(=O)c2ccc(-c4cccc5c4oc4ccccc45)cc2-3)OC1(C)C] | 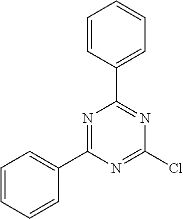 [SMILES: Clc1nc(-c2ccccc2)nc(-c2ccccc2)n1] |
| 38d | 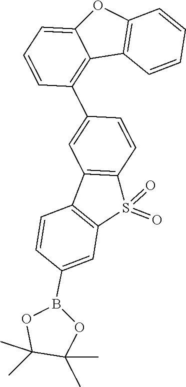 [SMILES: CC1(C)OB(c2ccc3c(c2)S(=O)(=O)c2ccc(-c4cccc5oc6ccccc6c45)cc2-3)OC1(C)C] | 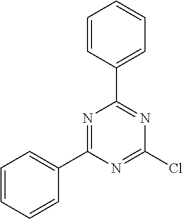 [SMILES: Clc1nc(-c2ccccc2)nc(-c2ccccc2)n1] |
| 39d | 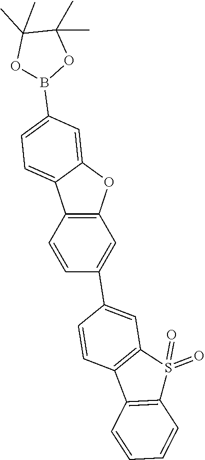 [SMILES: CC1(C)OB(c2ccc3c(c2)oc2cc(-c4ccc5c(c4)S(=O)(=O)c4ccccc4-5)ccc23)OC1(C)C] | 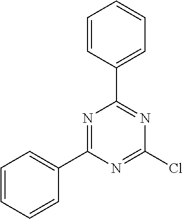 [SMILES: Clc1nc(-c2ccccc2)nc(-c2ccccc2)n1] |
| 40d | 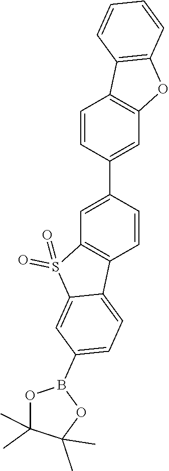 [SMILES: CC1(C)OB(c2ccc3c(c2)S(=O)(=O)c2cc(-c4ccc5c(c4)oc4ccccc45)ccc2-3)OC1(C)C] | 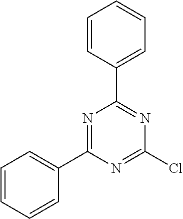 [SMILES: Clc1nc(-c2ccccc2)nc(-c2ccccc2)n1] |
| 41d | 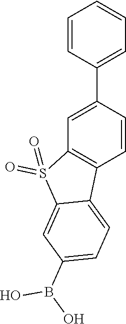 [SMILES: O=S1(=O)c2cc(B(O)O)ccc2-c2ccc(-c3ccccc3)cc21] | 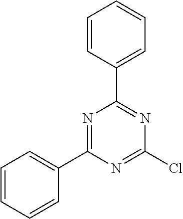 [SMILES: Clc1nc(-c2ccccc2)nc(-c2ccccc2)n1] |
| 42d | 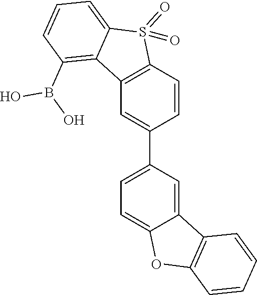 [SMILES: O=S1(=O)c2ccc(-c3ccc4oc5ccccc5c4c3)cc2-c2c(B(O)O)cccc21] | 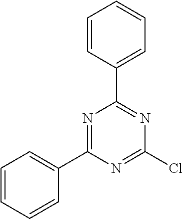 [SMILES: Clc1nc(-c2ccccc2)nc(-c2ccccc2)n1] |
| 42d | 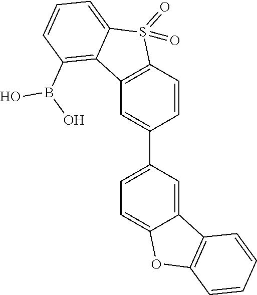 [SMILES: O=S1(=O)c2ccc(-c3ccc4oc5ccccc5c4c3)cc2-c2c(B(O)O)cccc21] | 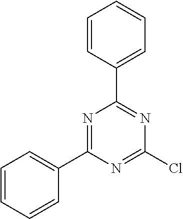 [SMILES: Clc1nc(-c2ccccc2)nc(-c2ccccc2)n1] |
| 43d | 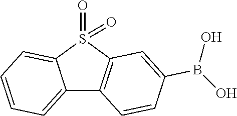 [SMILES: O=S1(=O)c2ccccc2-c2ccc(B(O)O)cc21] | 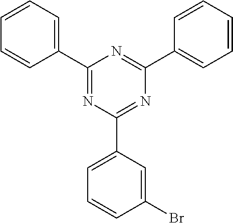 [SMILES: Brc1cccc(-c2nc(-c3ccccc3)nc(-c3ccccc3)n2)c1] |
| 44d | 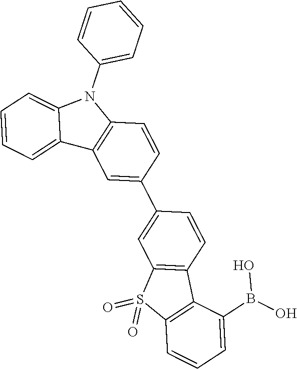 [SMILES: O=S1(=O)c2cc(-c3ccc4c(c3)c3ccccc3n4-c3ccccc3)ccc2-c2c(B(O)O)cccc21] | 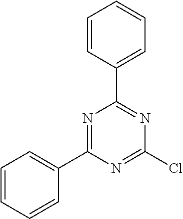 [SMILES: Clc1nc(-c2ccccc2)nc(-c2ccccc2)n1] |
| 45d | 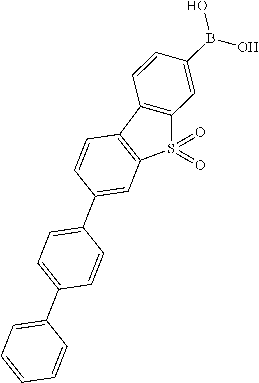 [SMILES: O=S1(=O)c2cc(B(O)O)ccc2-c2ccc(-c3ccc(-c4ccccc4)cc3)cc21] | 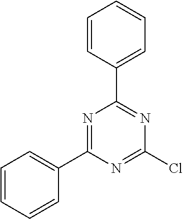 [SMILES: Clc1nc(-c2ccccc2)nc(-c2ccccc2)n1] |

| | Product | Yield |
|---|---|---|
| 1d | 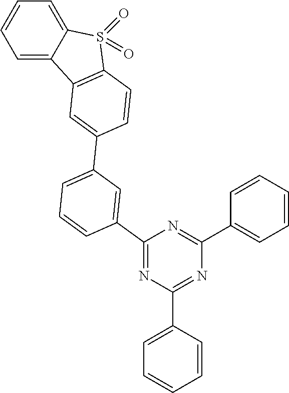 [SMILES: O=S1(=O)c2ccccc2-c2cc(-c3cccc(-c4nc(-c5ccccc5)nc(-c5ccccc5)n4)c3)ccc21] | 79% |
| 2d | 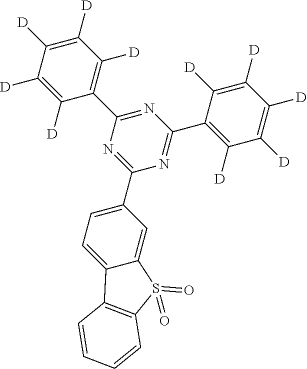 [SMILES: [2H]c1c([2H])c([2H])c(-c2nc(-c3ccc4c(c3)S(=O)(=O)c3ccccc3-4)nc(-c3c([2H])c([2H])c([2H])c([2H])c3[2H])n2)c([2H])c1[2H]] | 78% |
| 3d | 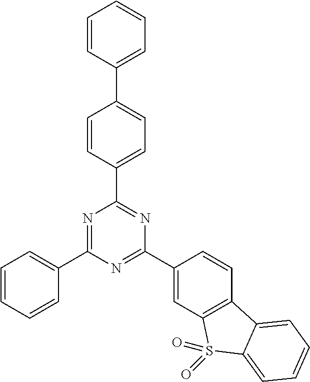 [SMILES: O=S1(=O)c2ccccc2-c2ccc(-c3nc(-c4ccccc4)nc(-c4ccc(-c5ccccc5)cc4)n3)cc21] | 72% |
| 4d | 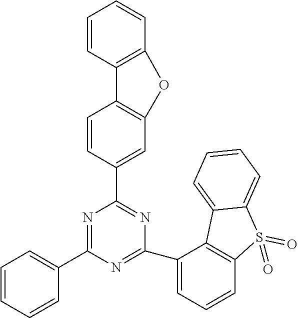 [SMILES: O=S1(=O)c2ccccc2-c2c(-c3nc(-c4ccccc4)nc(-c4ccc5c(c4)oc4ccccc45)n3)cccc21] | 70% |
| 5d | 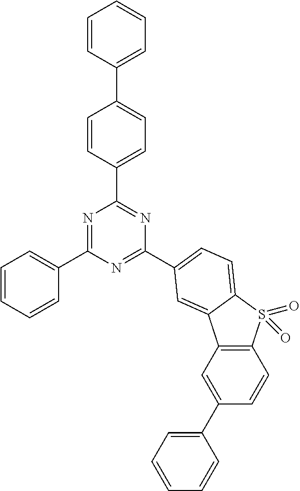 [SMILES: O=S1(=O)c2ccc(-c3ccccc3)cc2-c2cc(-c3nc(-c4ccccc4)nc(-c4ccc(-c5ccccc5)cc4)n3)ccc21] | 75% |
| 6d | 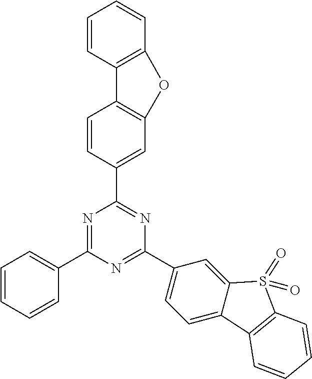 [SMILES: O=S1(=O)c2ccccc2-c2ccc(-c3nc(-c4ccccc4)nc(-c4ccc5c(c4)oc4ccccc45)n3)cc21] | 86% |
| 7d | 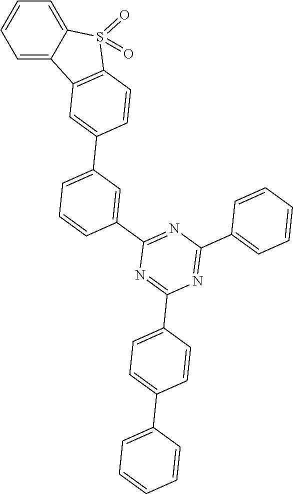 [SMILES: O=S1(=O)c2ccccc2-c2cc(-c3cccc(-c4nc(-c5ccccc5)nc(-c5ccc(-c6ccccc6)cc5)n4)c3)ccc21] | 78% |
| 8d | 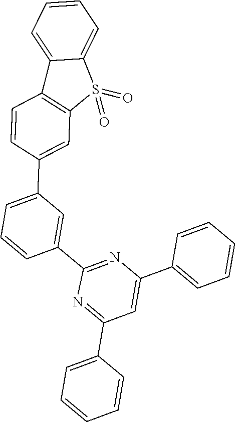 [SMILES: O=S1(=O)c2ccccc2-c2ccc(-c3cccc(-c4nc(-c5ccccc5)cc(-c5ccccc5)n4)c3)cc21] | 77% |
| 9d | 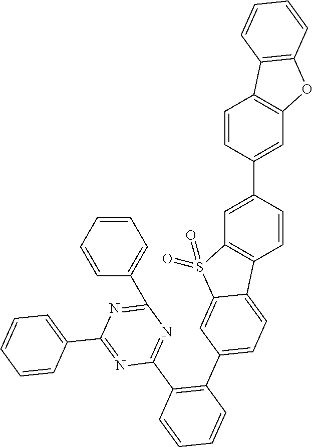 [SMILES: O=S1(=O)c2cc(-c3ccc4c(c3)oc3ccccc34)ccc2-c2ccc(-c3ccccc3-c3nc(-c4ccccc4)nc(-c4ccccc4)n3)cc21] | 71% |
| 10d | 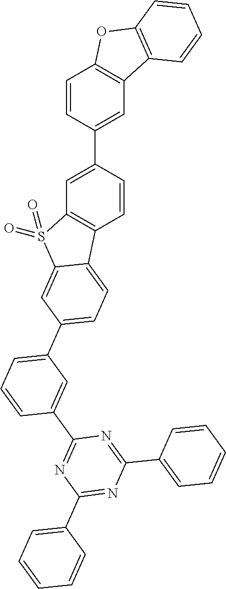 [SMILES: O=S1(=O)c2cc(-c3cccc(-c4nc(-c5ccccc5)nc(-c5ccccc5)n4)c3)ccc2-c2ccc(-c3ccc4oc5ccccc5c4c3)cc21] | 78% |
| 11d | 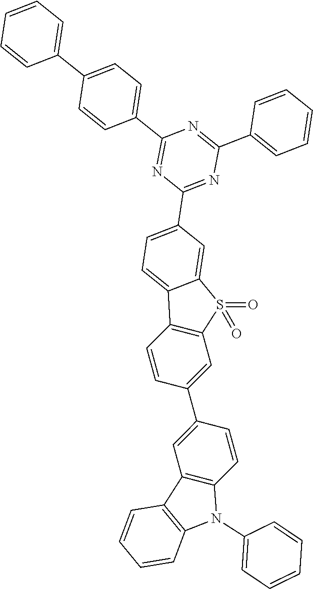 [SMILES: O=S1(=O)c2cc(-c3ccc4c(c3)c3ccccc3n4-c3ccccc3)ccc2-c2ccc(-c3nc(-c4ccccc4)nc(-c4ccc(-c5ccccc5)cc4)n3)cc21] | 83% |
| 12d | 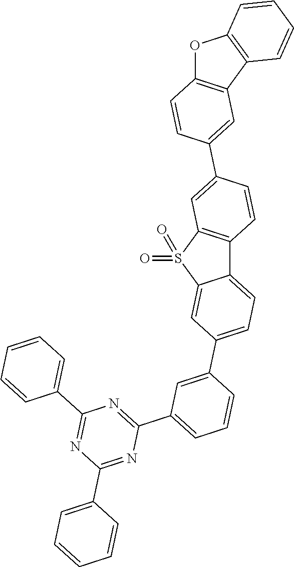 [SMILES: O=S1(=O)c2cc(-c3cccc(-c4nc(-c5ccccc5)nc(-c5ccccc5)n4)c3)ccc2-c2ccc(-c3ccc4oc5ccccc5c4c3)cc21] | 81% |
| 13d | 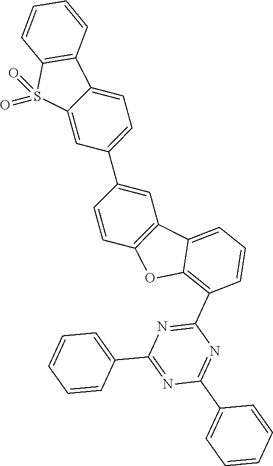 [SMILES: O=S1(=O)c2ccccc2-c2ccc(-c3ccc4oc5c(-c6nc(-c7ccccc7)nc(-c7ccccc7)n6)cccc5c4c3)cc21] | 85% |
| 14d | 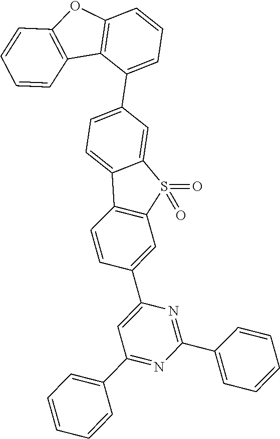 [SMILES: O=S1(=O)c2cc(-c3cc(-c4ccccc4)nc(-c4ccccc4)n3)ccc2-c2ccc(-c3cccc4oc5ccccc5c34)cc21] | 82% |
| 15d | 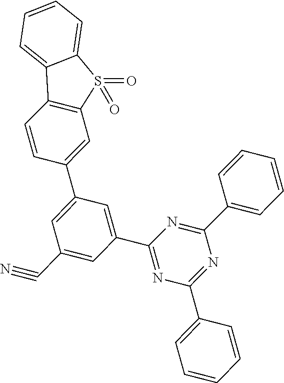 [SMILES: N#Cc1cc(-c2ccc3c(c2)S(=O)(=O)c2ccccc2-3)cc(-c2nc(-c3ccccc3)nc(-c3ccccc3)n2)c1] | 77% |
| 16d | 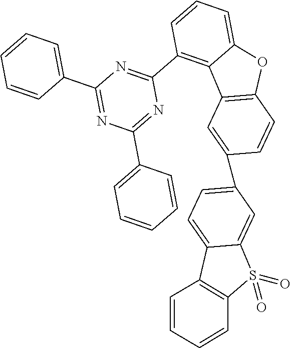 [SMILES: O=S1(=O)c2ccccc2-c2ccc(-c3ccc4oc5cccc(-c6nc(-c7ccccc7)nc(-c7ccccc7)n6)c5c4c3)cc21] | 76% |
| 17d | 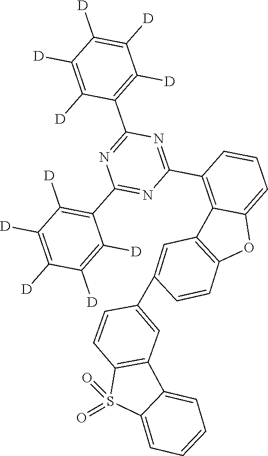 [SMILES: [2H]c1c([2H])c([2H])c(-c2nc(-c3c([2H])c([2H])c([2H])c([2H])c3[2H])nc(-c3cccc4oc5ccc(-c6ccc7c(c6)-c6ccccc6S7(=O)=O)cc5c34)n2)c([2H])c1[2H]] | 68% |
| 18d | 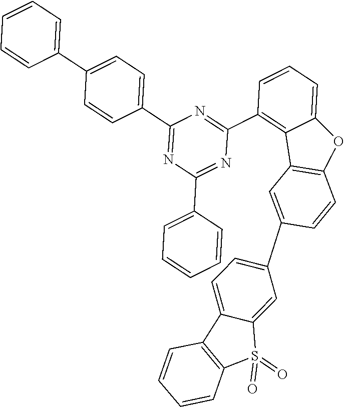 [SMILES: O=S1(=O)c2ccccc2-c2ccc(-c3ccc4oc5cccc(-c6nc(-c7ccccc7)nc(-c7ccc(-c8ccccc8)cc7)n6)c5c4c3)cc21] | 81% |
| 19d | 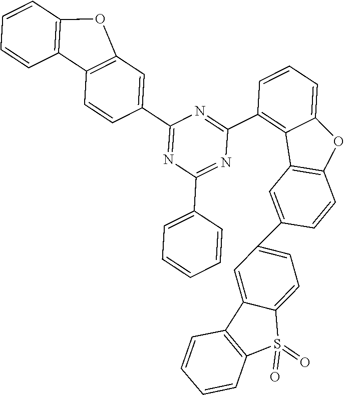 [SMILES: O=S1(=O)c2ccccc2-c2cc(-c3ccc4oc5cccc(-c6nc(-c7ccccc7)nc(-c7ccc8c(c7)oc7ccccc78)n6)c5c4c3)ccc21] | 80% |
| 20d | 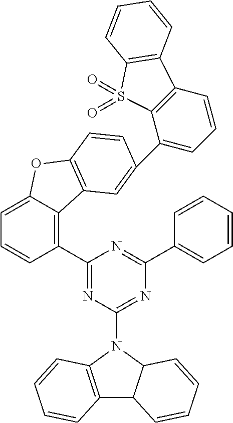 [SMILES: O=S1(=O)c2ccccc2-c2cccc(-c3ccc4oc5cccc(-c6nc(-c7ccccc7)nc(N7c8ccccc8C8C=CC=CC87)n6)c5c4c3)c21] | 74% |
| 21d | 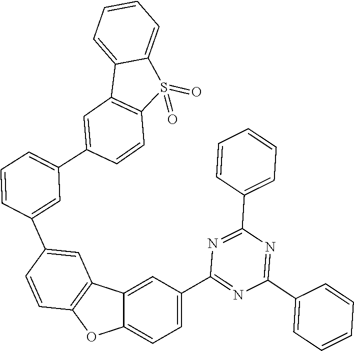 [SMILES: O=S1(=O)c2ccccc2-c2cc(-c3cccc(-c4ccc5oc6ccc(-c7nc(-c8ccccc8)nc(-c8ccccc8)n7)cc6c5c4)c3)ccc21] | 79% |
| 22d | 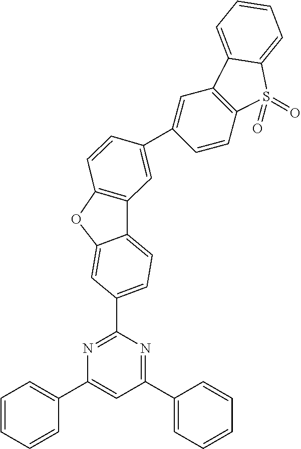 [SMILES: O=S1(=O)c2ccccc2-c2cc(-c3ccc4oc5cc(-c6nc(-c7ccccc7)cc(-c7ccccc7)n6)ccc5c4c3)ccc21] | 85% |
| 23d | 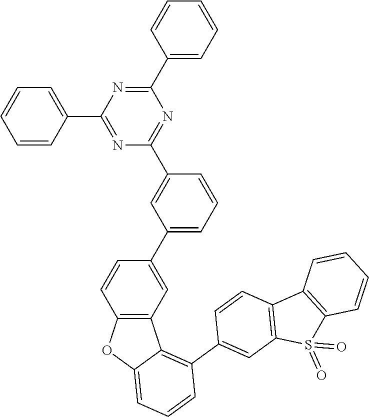 [SMILES: O=S1(=O)c2ccccc2-c2ccc(-c3cccc4oc5ccc(-c6cccc(-c7nc(-c8ccccc8)nc(-c8ccccc8)n7)c6)cc5c34)cc21] | 80% |
| 24d | 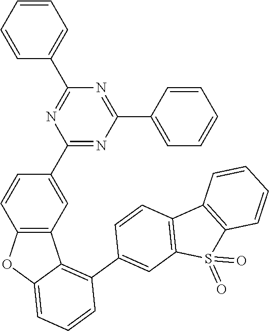 [SMILES: O=S1(=O)c2ccccc2-c2ccc(-c3cccc4oc5ccc(-c6nc(-c7ccccc7)nc(-c7ccccc7)n6)cc5c34)cc21] | 82% |
| 25d | 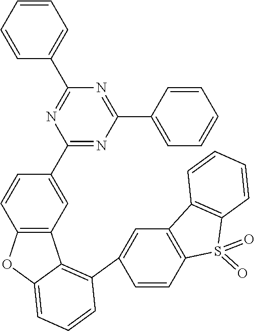 [SMILES: O=S1(=O)c2ccccc2-c2cc(-c3cccc4oc5ccc(-c6nc(-c7ccccc7)nc(-c7ccccc7)n6)cc5c34)ccc21] | 75% |
| 25d | 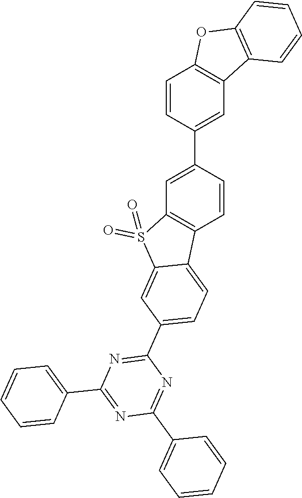 [SMILES: O=S1(=O)c2cc(-c3ccc4oc5ccccc5c4c3)ccc2-c2ccc(-c3nc(-c4ccccc4)nc(-c4ccccc4)n3)cc21] | 71% |
| 26d | 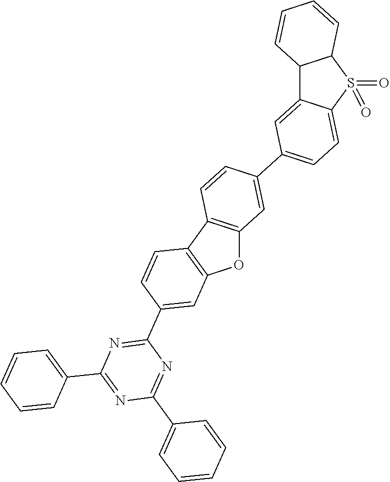 [SMILES: O=S1(=O)c2ccc(-c3ccc4c(c3)oc3cc(-c5nc(-c6ccccc6)nc(-c6ccccc6)n5)ccc34)cc2C2C=CC=CC21] | 67% |
| 27d | 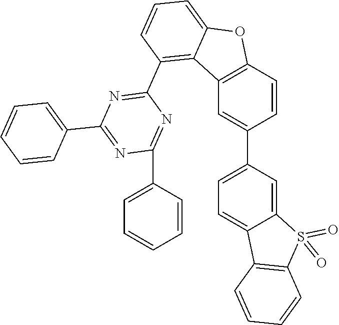 [SMILES: O=S1(=O)c2ccccc2-c2ccc(-c3ccc4oc5cccc(-c6nc(-c7ccccc7)nc(-c7ccccc7)n6)c5c4c3)cc21] | 64% |
| 28d | 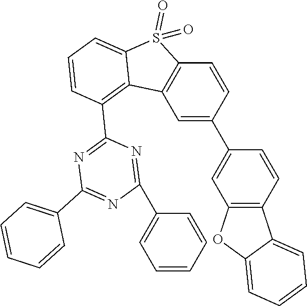 [SMILES: O=S1(=O)c2ccc(-c3ccc4c(c3)oc3ccccc34)cc2-c2c(-c3nc(-c4ccccc4)nc(-c4ccccc4)n3)cccc21] | 60% |
| 29d | 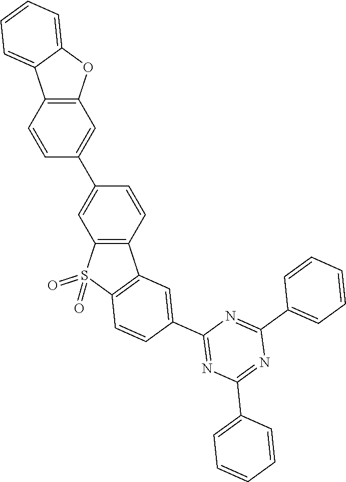 [SMILES: O=S1(=O)c2ccc(-c3nc(-c4ccccc4)nc(-c4ccccc4)n3)cc2-c2ccc(-c3ccc4c(c3)oc3ccccc34)cc21] | 64% |
| 30d | 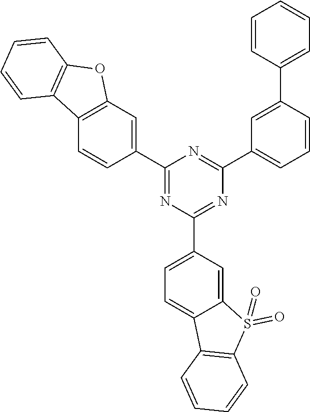 [SMILES: O=S1(=O)c2ccccc2-c2ccc(-c3nc(-c4cccc(-c5ccccc5)c4)nc(-c4ccc5c(c4)oc4ccccc45)n3)cc21] | 63% |
| 31d | 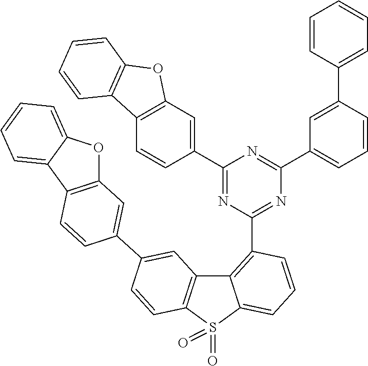 [SMILES: O=S1(=O)c2ccc(-c3ccc4c(c3)oc3ccccc34)cc2-c2c(-c3nc(-c4cccc(-c5ccccc5)c4)nc(-c4ccc5c(c4)oc4ccccc45)n3)cccc21] | 60% |
| 32d | 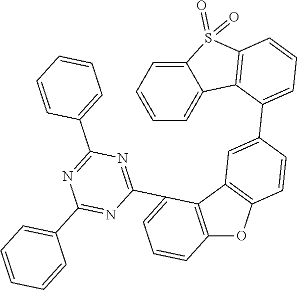 [SMILES: O=S1(=O)c2ccccc2-c2c(-c3ccc4oc5cccc(-c6nc(-c7ccccc7)nc(-c7ccccc7)n6)c5c4c3)cccc21] | 65% |
| 33d | 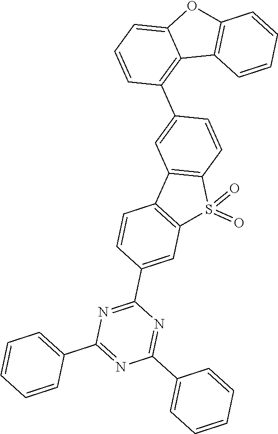 [SMILES: O=S1(=O)c2ccc(-c3cccc4oc5ccccc5c34)cc2-c2ccc(-c3nc(-c4ccccc4)nc(-c4ccccc4)n3)cc21] | 62% |
| 34d | 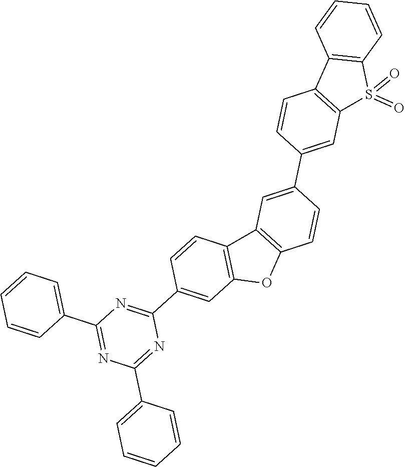 [SMILES: O=S1(=O)c2ccccc2-c2ccc(-c3ccc4oc5cc(-c6nc(-c7ccccc7)nc(-c7ccccc7)n6)ccc5c4c3)cc21] | 60% |
| 35d | 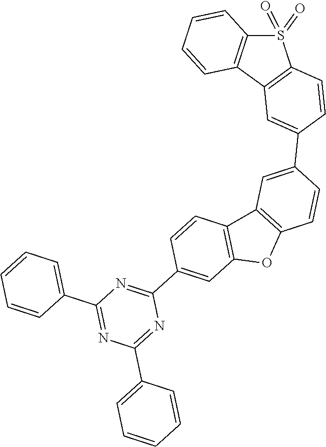 [SMILES: O=S1(=O)c2ccccc2-c2cc(-c3ccc4oc5cc(-c6nc(-c7ccccc7)nc(-c7ccccc7)n6)ccc5c4c3)ccc21] | 58% |
| 36d | 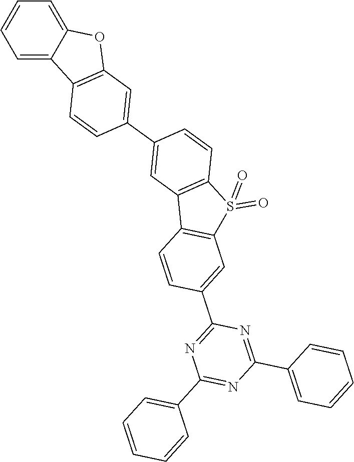 [SMILES: O=S1(=O)c2ccc(-c3ccc4c(c3)oc3ccccc34)cc2-c2ccc(-c3nc(-c4ccccc4)nc(-c4ccccc4)n3)cc21] | 70% |
| 37d | 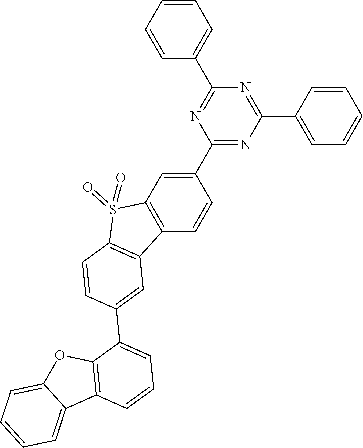 [SMILES: O=S1(=O)c2ccc(-c3cccc4c3oc3ccccc34)cc2-c2ccc(-c3nc(-c4ccccc4)nc(-c4ccccc4)n3)cc21] | 74% |
| 38d | 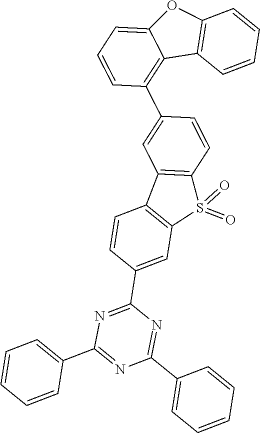 [SMILES: O=S1(=O)c2ccc(-c3cccc4oc5ccccc5c34)cc2-c2ccc(-c3nc(-c4ccccc4)nc(-c4ccccc4)n3)cc21] | 68% |
| 39d | 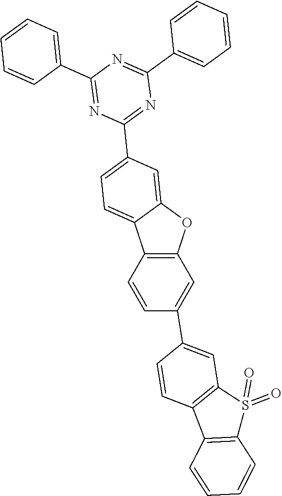 [SMILES: O=S1(=O)c2ccccc2-c2ccc(-c3ccc4c(c3)oc3cc(-c5nc(-c6ccccc6)nc(-c6ccccc6)n5)ccc34)cc21] | 71% |
| 40d | 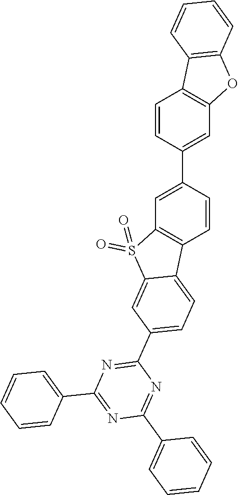 [SMILES: O=S1(=O)c2cc(-c3ccc4c(c3)oc3ccccc34)ccc2-c2ccc(-c3nc(-c4ccccc4)nc(-c4ccccc4)n3)cc21] | 65% |
| 41d | 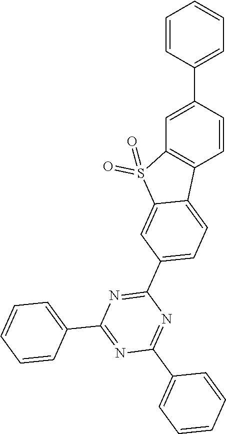 [SMILES: O=S1(=O)c2cc(-c3ccccc3)ccc2-c2ccc(-c3nc(-c4ccccc4)nc(-c4ccccc4)n3)cc21] | 60% |
| 42d | 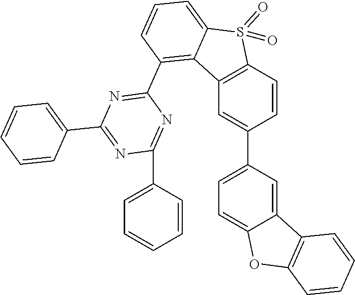 [SMILES: O=S1(=O)c2ccc(-c3ccc4oc5ccccc5c4c3)cc2-c2c(-c3nc(-c4ccccc4)nc(-c4ccccc4)n3)cccc21] | 65% |
| 42d | 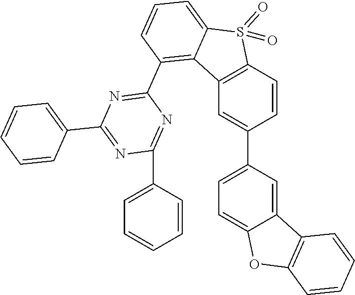 [SMILES: O=S1(=O)c2ccc(-c3ccc4oc5ccccc5c4c3)cc2-c2c(-c3nc(-c4ccccc4)nc(-c4ccccc4)n3)cccc21] | 65% |
| 43d | 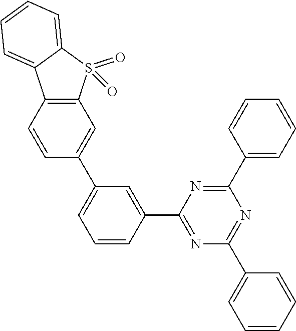 [SMILES: O=S1(=O)c2ccccc2-c2ccc(-c3cccc(-c4nc(-c5ccccc5)nc(-c5ccccc5)n4)c3)cc21] | 64% |
| 44d | 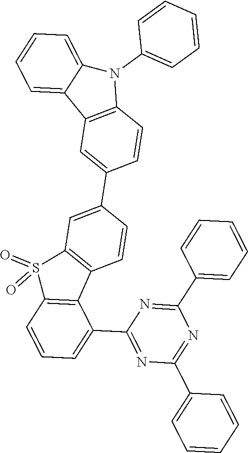 [SMILES: O=S1(=O)c2cc(-c3ccc4c(c3)c3ccccc3n4-c3ccccc3)ccc2-c2c(-c3nc(-c4ccccc4)nc(-c4ccccc4)n3)cccc21] | 60% |
| 45d | 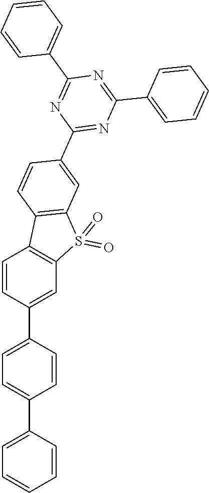 [SMILES: O=S1(=O)c2cc(-c3ccc(-c4ccccc4)cc3)ccc2-c2ccc(-c3nc(-c4ccccc4)nc(-c4ccccc4)n3)cc21] | 65% |

### Example 1: Production of the OLEDs

Examples E1 to E25 which follow present the use of the compounds of the invention in OLEDs.

Pretreatment for examples E1-E23: Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2. The data of the OLEDs are listed in table 3.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as EG1:IC2:TER5 (55%:35%:10%) mean here that the material EG1 is present in the layer in a proportion by volume of 55%, IC2 in a proportion by volume of 35% and TER5 in a proportion by volume of 10%. Analogously, the electron transport layer may also consist of a mixture of two materials.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. Electroluminescence spectra are determined at a luminance of 1000 cd/m², and these are used to calculate the CIE 1931 x and y color coordinates. The parameter U1000 in table 3 refers to the voltage which is required for a luminance of 1000 cd/m². EQE1000 denotes the external quantum efficiency which is attained at 1000 cd/m².

### Use of Mixtures of the Invention in OLEDs

With the use of the inventive compounds EG1 to EG11 and EG13 to EG15 in examples E1 to E12 and E14 to E19 as matrix material in the emission layer of phosphorescent green OLEDs and EG12 in example E13 as matrix material in the emission layer of phosphorescent red OLEDs, it can be shown that use in a mixture with a second host material IC2-IC4 achieves improved performance data of the OLEDs compared to the prior art (V1 to V12), particularly with regard to efficiency and voltage.

When the inventive compounds (EG1, EG2, EG9 and EG10) are used as electron transport material in examples E20 to E23, significantly lower voltage and better efficiency and lifetime are achieved than with the substance SdT1 and SdT2 according to the prior art (examples V14 and V15).

TABLE 1
Structure of the OLEDs

| | | HIL | HTL | EBL | EML | HBL | ETL | EIL |
|---|---|---|---|---|---|---|---|---|
| Ex. | IL | thickness | thickness | thickness | thickness | thickness | thickness | thickness |
| V1 | | HATCN | SpMA1 | SpMA3 | SdT1:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E1 | | HATCN | SpMA1 | SpMA3 | EG1:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V2 | | HATCN | SpMA1 | SpMA3 | SdT2:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E2 | | HATCN | SpMA1 | SpMA3 | EG2:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V3 | | HATCN | SpMA1 | SpMA3 | SdT3:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E3 | | HATCN | SpMA1 | SpMA3 | EG3:IC2:TEG1 | ST2 | ST2:LiQ | LiQ 1 nm |
| | | 5 nm | 125 nm | 10 nm | (97%:3%) 35 nm | 10 nm | (50%:50%) | |
| | | | | | | | 30 nm | |
| V4 | | HATCN | SpMA1 | SpMA3 | SdT4:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E4 | | HATCN | SpMA1 | SpMA3 | EG4:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V5 | | HATCN | SpMA1 | SpMA3 | SdT5:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E5 | | HATCN | SpMA1 | SpMA3 | EG5:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V6 | | HATCN | SpMA1 | SpMA3 | SdT6:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E6 | | HATCN | SpMA1 | SpMA3 | EG6:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V7 | | HATCN | SpMA1 | SpMA3 | SdT7:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E7 | | HATCN | SpMA1 | SpMA3 | EG7:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V8 | | HATCN | SpMA1 | SpMA3 | SdT8:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E8 | | HATCN | SpMA1 | SpMA3 | EG8:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V9 | | HATCN | SpMA1 | SpMA3 | SdT9:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E9 | | HATCN | SpMA1 | SpMA3 | EG9:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E10 | | HATCN | SpMA1 | SpMA3 | EG10:IC3: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E11 | | HATCN | SpMA1 | SpMA3 | EG11:IC3: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E12 | | HATCN | SpMA1 | SpMA3 | EG12:IC4:TEG1 | ST2 | ST2:LiQ | LiQ 1 nm |
| | | 5 nm | 125 nm | 10 nm | (46%:46%:8%) | 10 nm | (50%:50%) | |
| | | | | | 30 nm | | 30 nm | |
| E13 | | HATCN | SpMA1 | SpMA3 | EG12:IC4:TER1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E14 | | HATCN | SpMA1 | SpMA3 | EG13:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E15 | | HATCN | SpMA1 | SpMA3 | EG14:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V10 | | HATCN | SpMA1 | SpMA3 | SdT10:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E16 | | HATCN | SpMA1 | SpMA3 | EG11:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V11 | | HATCN | SpMA1 | SpMA3 | SdT11:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E17 | | HATCN | SpMA1 | SpMA3 | EG12:IC2: TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V12 | | HATCN | SpMA1 | SpMA3 | SdT12:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E18 | | HATCN | SpMA1 | SpMA3 | EG112:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V13 | | HATCN | SpMA1 | SpMA3 | EG15:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E19 | | HATCN | SpMA1 | SpMA3 | SdT13:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| V14 | SpA1 | | HATCN | SpMA1 | M2:SEB | — | SdT1:LiQ | — |
| | 140 nm | | 5 nm | 20 nm | (95%:5%) | | (50%:50%) | |
| | | | | | 20 nm | | 30 nm | |
| V15 | SpA1 | | HATCN | SpMA1 | IC1:TEG1 | IC1 | SdT2:LiQ | — |
| | 70 nm | | 5 nm | 90 nm | (90%:10%) | 10 nm | (50%:50%) | |
| | | | | | 30 nm | | 30 nm | |
| E20 | SpA1 | | HATCN | SpMA1 | M2:SEB | — | EG1:LiQ | — |
| | 140 nm | | 5 nm | 20 nm | (95%:5%) | | (50%:50%) | |
| | | | | | 20 nm | | 30 nm | |
| E21 | SpA1 | | HATCN | SpMA1 | IC1:TEG1 | IC1 | EG2:LiQ | — |
| | 70 nm | | 5 nm | 90 nm | (90%:10%) | 10 nm | (50%:50%) | |
| | | | | | 30 nm | | 30 nm | |
| E22 | SpA1 | | HATCN | SpMA1 | M2:SEB | — | EG9:LiQ | — |
| | 140 nm | | 5 nm | 20 nm | (95%:5%) | | (50%:50%) | |
| | | | | | 20 nm | | 30 nm | |
| E23 | SpA1 | | HATCN | SpMA1 | IC1:TEG1 | — | EG10:LiQ | — |
| | 70 nm | | 5 nm | 90 nm | (90%:10%) | | (50%:50%) | |
| | | | | | 30 nm | | 40 nm | |
| E24 | SpA1 | | HATCN | SpMA1 | IC5:TEG1 | — | EG10:LiQ | — |
| | 70 nm | | 5 nm | 90 nm | (90%:10%) | | (50%:50%) | |
| | | | | | 30 nm | | 40 nm | |
| E25 | SpA1 | | HATCN | SpMA1 | IC6:TEG1 | — | EG10:LiQ | — |
| | 70 nm | | 5 nm | 90 nm | (90%:10%) | | (50%:50%) | |
| | | | | | 30 nm | | 40 nm | |

TABLE 2
Structural formulae of the materials for OLEDs

| | |
|---|---|
| 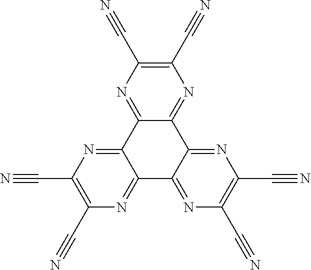 [SMILES: N#Cc1nc2c3nc(C#N)c(C#N)nc3c3nc(C#N)c(C#N)nc3c2nc1C#N] | 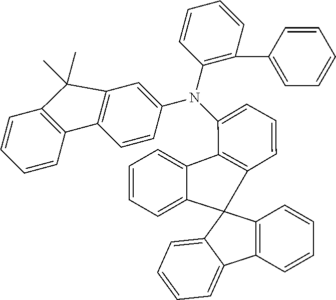 [SMILES: CC1(C)c2ccccc2-c2ccc(N(c3ccccc3-c3ccccc3)c3cccc4c3-c3ccccc3C43c4ccccc4-c4ccccc43)cc21] |
| HATCN | SpMA1 |
| 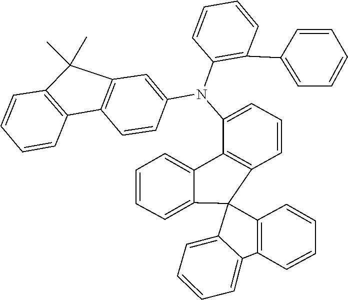 [SMILES: CC1(C)c2ccccc2-c2ccc(N(c3ccccc3-c3ccccc3)c3cccc4c3-c3ccccc3C43c4ccccc4-c4ccccc43)cc21] | 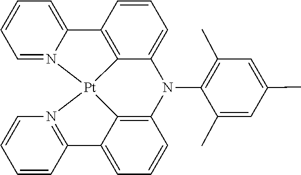 |
| SpMA3 | TER1 |
| 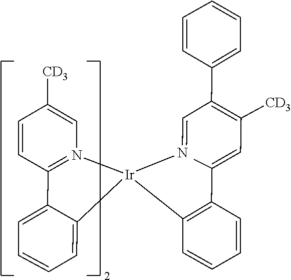 | 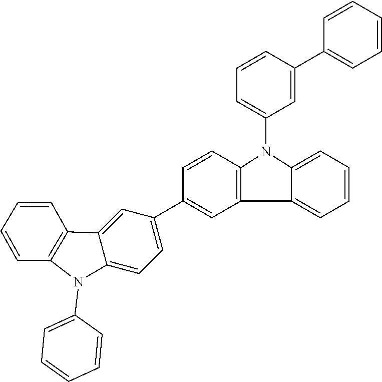 [SMILES: c1ccc(-c2cccc(-n3c4ccccc4c4cc(-c5ccc6c(c5)c5ccccc5n6-c5ccccc5)ccc43)c2)cc1] |
| TEG1 | IC2 |
| 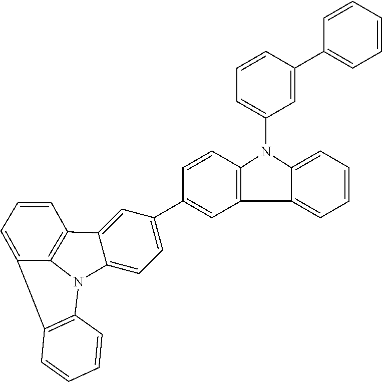 [SMILES: c1ccc(-c2cccc(-n3c4ccccc4c4cc(-c5ccc6c(c5)c5cccc7c8ccccc8n6c75)ccc43)c2)cc1] | 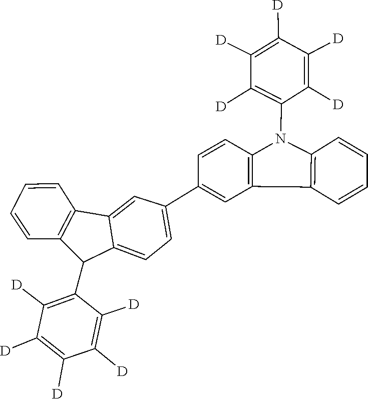 [SMILES: [2H]c1c([2H])c([2H])c(C2c3ccccc3-c3cc(-c4ccc5c(c4)c4ccccc4n5-c4c([2H])c([2H])c([2H])c([2H])c4[2H])ccc32)c([2H])c1[2H]] |
| IC3 | IC4 |
| 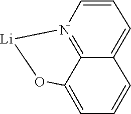 | 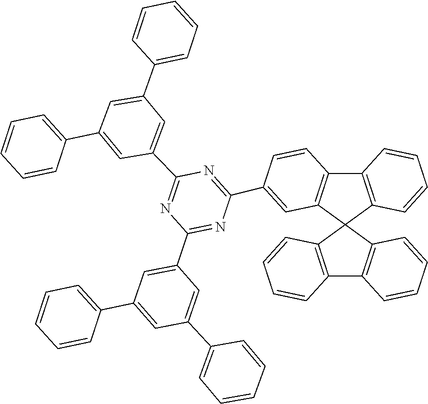 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] |
| LiQ | ST2 |
| 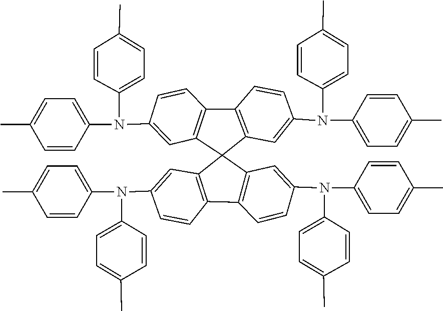 | 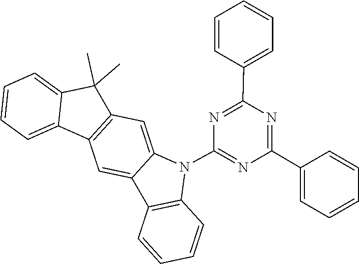 [SMILES: CC1(C)c2ccccc2-c2cc3c4ccccc4n(-c4nc(-c5ccccc5)nc(-c5ccccc5)n4)c3cc21] |
| SpA1 | IC1 |
| 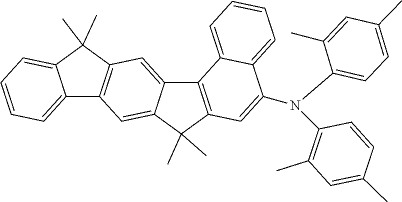 [SMILES: Cc1ccc(N(c2ccc(C)cc2C)c2cc3c(c4ccccc24)-c2cc4c(cc2C3(C)C)-c2ccccc2C4(C)C)c(C)c1] | 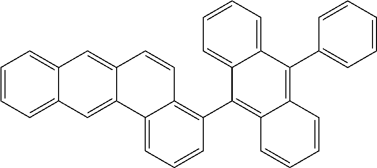 [SMILES: c1ccc(-c2c3ccccc3c(-c3cccc4c3ccc3cc5ccccc5cc34)c3ccccc23)cc1] |
| SEB | M2 |
| 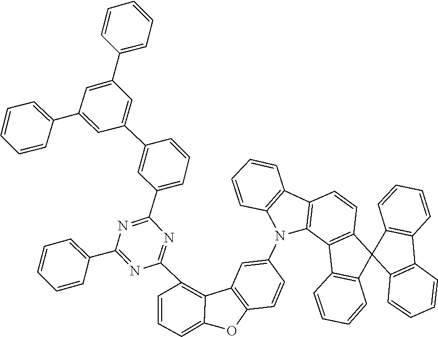 [SMILES: c1ccc(-c2cc(-c3ccccc3)cc(-c3cccc(-c4nc(-c5ccccc5)nc(-c5cccc6oc7ccc(-n8c9ccccc9c9ccc%10c(c98)-c8ccccc8C%108c9ccccc9-c9ccccc98)cc7c56)n4)c3)c2)cc1] | 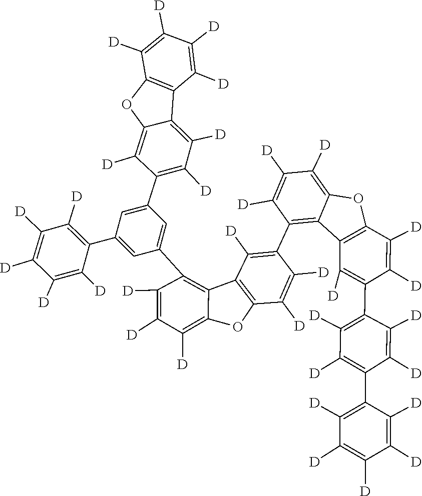 [SMILES: [2H]c1c([2H])c([2H])c(-c2cc(-c3c([2H])c([2H])c4c(oc5c([2H])c([2H])c([2H])c([2H])c54)c3[2H])cc(-c3c([2H])c([2H])c([2H])c4oc5c([2H])c([2H])c(-c6c([2H])c([2H])c([2H])c7oc8c([2H])c([2H])c(-c9c([2H])c([2H])c(-c%10c([2H])c([2H])c([2H])c([2H])c%10[2H])c([2H])c9[2H])c([2H])c8c67)c([2H])c5c34)c2)c([2H])c1[2H]] |
| IC5 | IC6 |
| 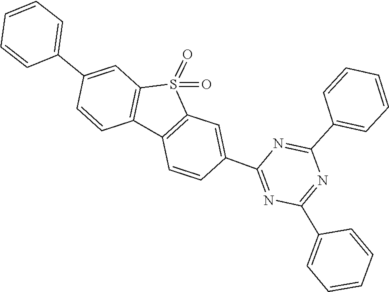 [SMILES: O=S1(=O)c2cc(-c3ccccc3)ccc2-c2ccc(-c3nc(-c4ccccc4)nc(-c4ccccc4)n3)cc21] | 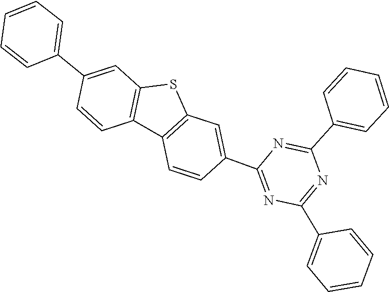 [SMILES: c1ccc(-c2ccc3c(c2)sc2cc(-c4nc(-c5ccccc5)nc(-c5ccccc5)n4)ccc23)cc1] 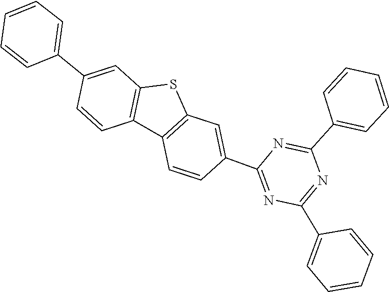 [SMILES: c1ccc(-c2ccc3c(c2)sc2cc(-c4nc(-c5ccccc5)nc(-c5ccccc5)n4)ccc23)cc1] |
| 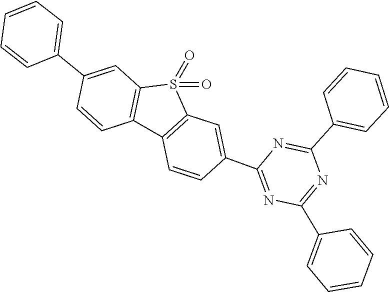 [SMILES: O=S1(=O)c2cc(-c3ccccc3)ccc2-c2ccc(-c3nc(-c4ccccc4)nc(-c4ccccc4)n3)cc21] | 2302038-20-4 |
| EG1 (41d) | SdT1 (WO2019054833) |
| 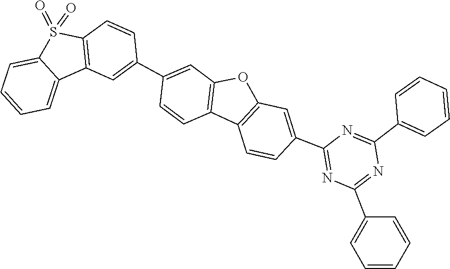 [SMILES: O=S1(=O)c2ccccc2-c2cc(-c3ccc4c(c3)oc3cc(-c5nc(-c6ccccc6)nc(-c6ccccc6)n5)ccc34)ccc21] | 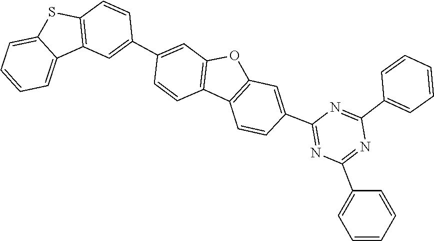 [SMILES: c1ccc(-c2nc(-c3ccccc3)nc(-c3ccc4c(c3)oc3cc(-c5ccc6sc7ccccc7c6c5)ccc34)n2)cc1] 2302034-69-9 |
| EG2 (26d) | SdT2 (WO2021071247) |
| 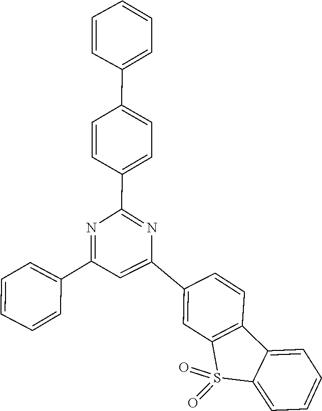 [SMILES: O=S1(=O)c2ccccc2-c2ccc(-c3cc(-c4ccccc4)nc(-c4ccc(-c5ccccc5)cc4)n3)cc21] | 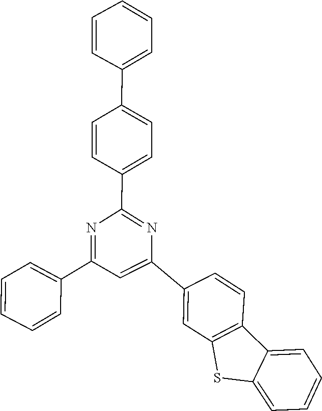 [SMILES: c1ccc(-c2ccc(-c3nc(-c4ccccc4)cc(-c4ccc5c(c4)sc4ccccc45)n3)cc2)cc1] 2360831-91-8 |
| EG3 (3d) | SdT3 (US2019198775) |
| 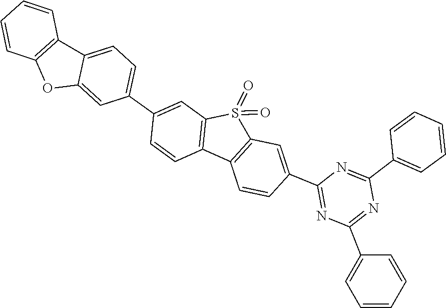 [SMILES: O=S1(=O)c2cc(-c3ccc4c(c3)oc3ccccc34)ccc2-c2ccc(-c3nc(-c4ccccc4)nc(-c4ccccc4)n3)cc21] | 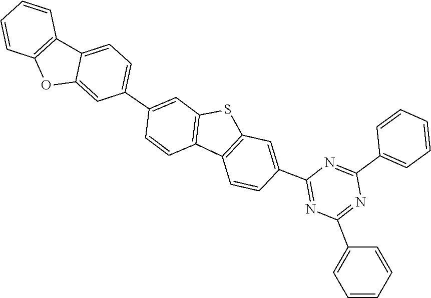 [SMILES: c1ccc(-c2nc(-c3ccccc3)nc(-c3ccc4c(c3)sc3cc(-c5ccc6c(c5)oc5ccccc56)ccc34)n2)cc1] 2302038-52-2 |
| EG4 (40d) | SdT4 (WO2019054833) |
| 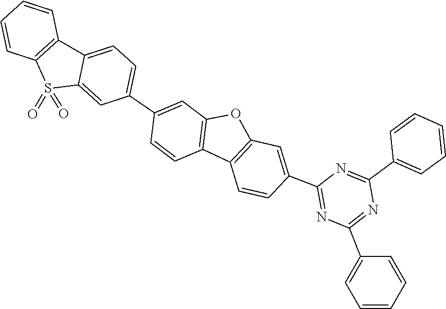 [SMILES: O=S1(=O)c2ccccc2-c2ccc(-c3ccc4c(c3)oc3cc(-c5nc(-c6ccccc6)nc(-c6ccccc6)n5)ccc34)cc21] | 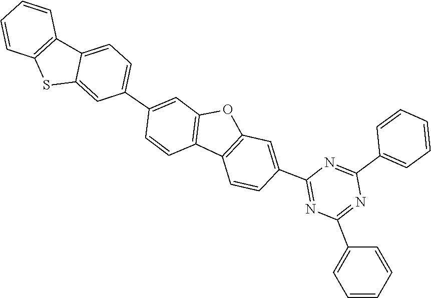 [SMILES: c1ccc(-c2nc(-c3ccccc3)nc(-c3ccc4c(c3)oc3cc(-c5ccc6c(c5)sc5ccccc56)ccc34)n2)cc1] 2302034-71-3 |
| EG5 (39d) | SdT5 (WO2019054833) |
| 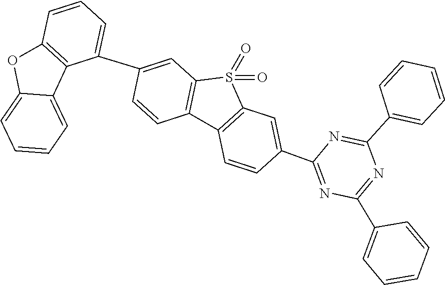 [SMILES: O=S1(=O)c2cc(-c3nc(-c4ccccc4)nc(-c4ccccc4)n3)ccc2-c2ccc(-c3cccc4oc5ccccc5c34)cc21] | 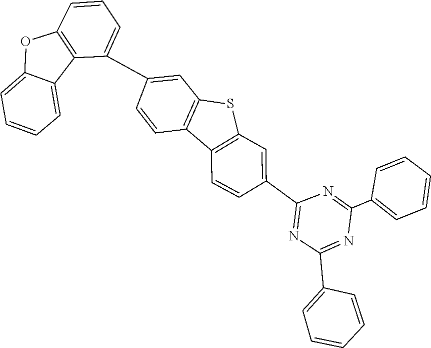 [SMILES: c1ccc(-c2nc(-c3ccccc3)nc(-c3ccc4c(c3)sc3cc(-c5cccc6oc7ccccc7c56)ccc34)n2)cc1] 2302038-54-4 |
| EG6 (38d) | SdT6 (WO2019054833) |
| 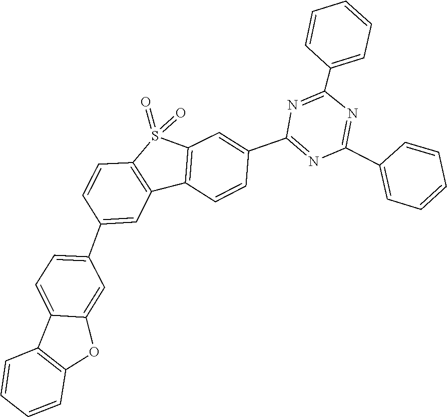 [SMILES: O=S1(=O)c2ccc(-c3ccc4c(c3)oc3ccccc34)cc2-c2ccc(-c3nc(-c4ccccc4)nc(-c4ccccc4)n3)cc21] | 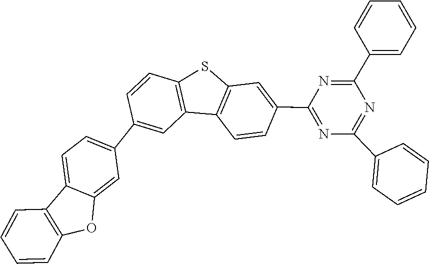 [SMILES: c1ccc(-c2nc(-c3ccccc3)nc(-c3ccc4c(c3)sc3ccc(-c5ccc6c(c5)oc5ccccc56)cc34)n2)cc1] 2302033-56-1 |
| EG7 (36d) | SdT7 (WO2019054833) |
| 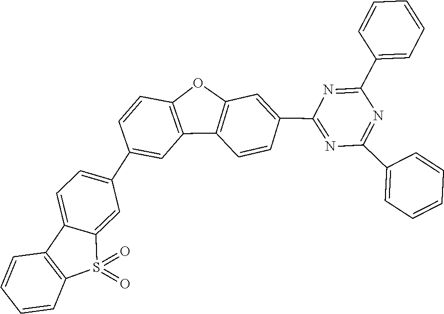 [SMILES: O=S1(=O)c2ccccc2-c2ccc(-c3ccc4oc5cc(-c6nc(-c7ccccc7)nc(-c7ccccc7)n6)ccc5c4c3)cc21] | 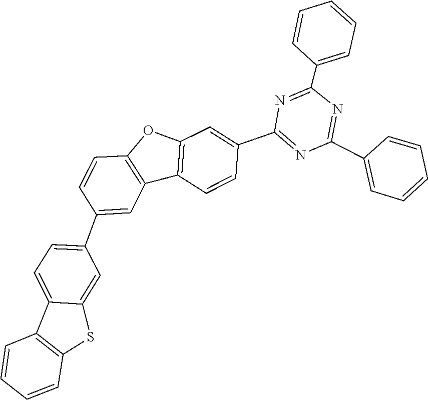 [SMILES: c1ccc(-c2nc(-c3ccccc3)nc(-c3ccc4c(c3)oc3ccc(-c5ccc6c(c5)sc5ccccc56)cc34)n2)cc1] 2302032-61-5 |
| EG8 (34d) | SdT8 (WO2021071247) |
| 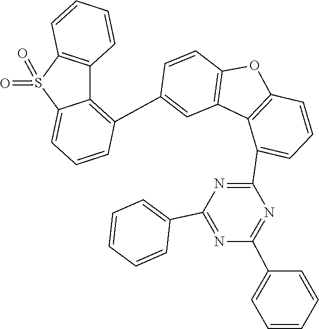 [SMILES: O=S1(=O)c2ccccc2-c2c(-c3ccc4oc5cccc(-c6nc(-c7ccccc7)nc(-c7ccccc7)n6)c5c4c3)cccc21] | 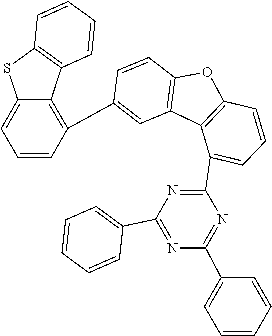 [SMILES: c1ccc(-c2nc(-c3ccccc3)nc(-c3cccc4oc5ccc(-c6cccc7sc8ccccc8c67)cc5c34)n2)cc1] 1822311-07-8 |
| EG9 (32d) | SdT9 (WO2015169412) |
| 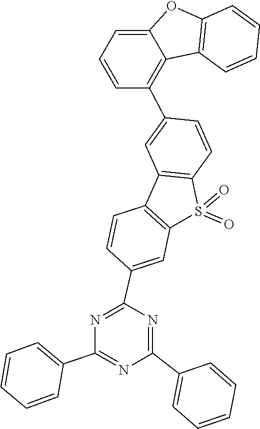 [SMILES: O=S1(=O)c2ccc(-c3cccc4oc5ccccc5c34)cc2-c2ccc(-c3nc(-c4ccccc4)nc(-c4ccccc4)n3)cc21] | 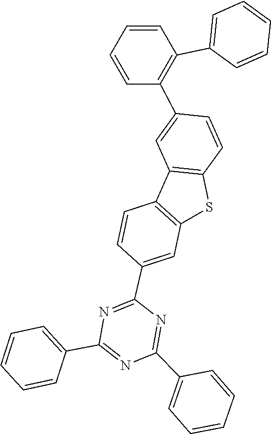 [SMILES: c1ccc(-c2nc(-c3ccccc3)nc(-c3ccc4c(c3)sc3ccc(-c5ccccc5-c5ccccc5)cc34)n2)cc1] 2302033-68-5 |
| EG10 (28d) | SdT10 (WO19054833 A1) |
| 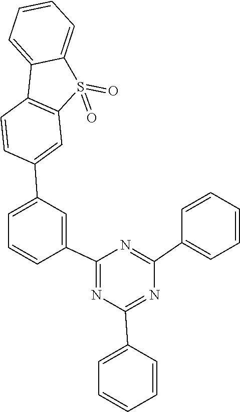 [SMILES: O=S1(=O)c2ccccc2-c2ccc(-c3cccc(-c4nc(-c5ccccc5)nc(-c5ccccc5)n4)c3)cc21] | 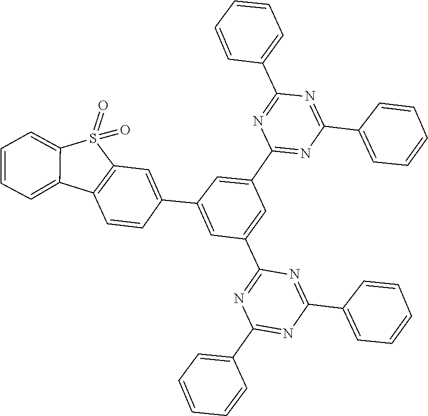 [SMILES: O=S1(=O)c2ccccc2-c2ccc(-c3cc(-c4nc(-c5ccccc5)nc(-c5ccccc5)n4)cc(-c4nc(-c5ccccc5)nc(-c5ccccc5)n4)c3)cc21] 2375083-26-2 |
| EG11 (43d) | SdT11 (WO 2019/164301) |
| 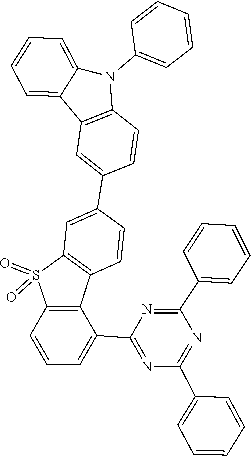 [SMILES: O=S1(=O)c2cc(-c3ccc4c(c3)c3ccccc3n4-c3ccccc3)ccc2-c2c(-c3nc(-c4ccccc4)nc(-c4ccccc4)n3)cccc21] | 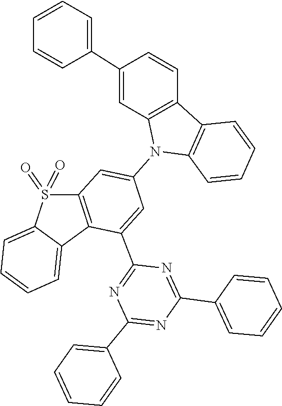 [SMILES: O=S1(=O)c2ccccc2-c2c(-c3nc(-c4ccccc4)nc(-c4ccccc4)n3)cc(-n3c4ccccc4c4ccc(-c5ccccc5)cc43)cc21] 2446251-38-1 |
| EG12 (44d) | SdT12 (CN110790756 B) |
| 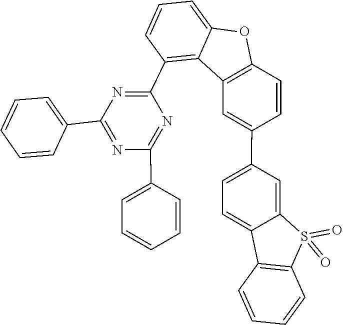 [SMILES: O=S1(=O)c2ccccc2-c2ccc(-c3ccc4oc5cccc(-c6nc(-c7ccccc7)nc(-c7ccccc7)n6)c5c4c3)cc21] | 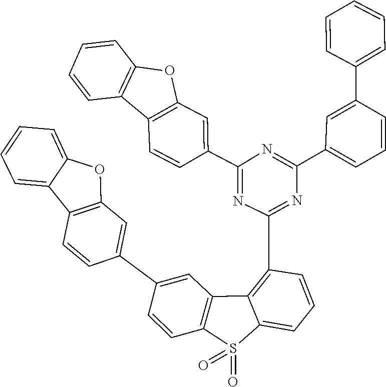 [SMILES: O=S1(=O)c2ccc(-c3ccc4c(c3)oc3ccccc34)cc2-c2c(-c3nc(-c4cccc(-c5ccccc5)c4)nc(-c4ccc5c(c4)oc4ccccc45)n3)cccc21] |
| EG13 (27d) | EG14 (d31) |
| 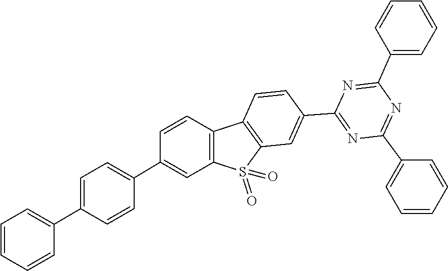 [SMILES: O=S1(=O)c2cc(-c3ccc(-c4ccccc4)cc3)ccc2-c2ccc(-c3nc(-c4ccccc4)nc(-c4ccccc4)n3)cc21] | 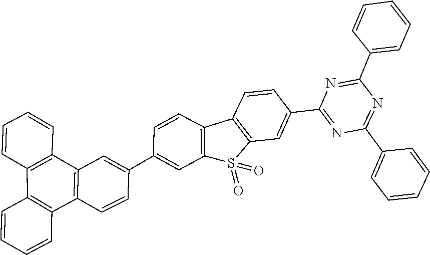 [SMILES: O=S1(=O)c2cc(-c3ccc4c5ccccc5c5ccccc5c4c3)ccc2-c2ccc(-c3nc(-c4ccccc4)nc(-c4ccccc4)n3)cc21] |
| EG15 (45d) | SdT13 (KR2015031396) |

TABLE 3
Data of the green OLEDs

| | | EQE | CIE x/y | | | |
|---|---|---|---|---|---|---|
| | U1000 | 1000 | at 1000 | j₀ | L1 | LT |
| Ex. | (V) | (%) | cd/m² | (mA/cm²) | (%) | (h) |
| V1 | 3.8 | 15 | 0.32/0.63 | 20 | 95 | 480 |
| E1 | 3.3 | 18 | 0.33/0.63 | 20 | 95 | 500 |
| V2 | 3.7 | 16 | 0.32/0.64 | 20 | 95 | 476 |
| E2 | 3.2 | 20 | 0.32/0.63 | 20 | 95 | 468 |
| V3 | 3.8 | 16 | 0.32/0.64 | 20 | 95 | 430 |
| E3 | 3.1 | 19 | 0.34/0.63 | 20 | 95 | 422 |
| V4 | 3.8 | 17 | 0.32/0.64 | 20 | 95 | 500 |
| E4 | 3.2 | 20 | 0.32/0.63 | 20 | 95 | 482 |
| V5 | 3.7 | 15 | 0.32/0.64 | 20 | 95 | 487 |
| E5 | 3.1 | 18 | 0.33/0.63 | 20 | 95 | 469 |
| V6 | 3.6 | 17 | 0.32/0.64 | 20 | 95 | 470 |
| E6 | 3.3 | 20 | 0.32/0.63 | 20 | 95 | 460 |
| V7 | 3.7 | 16 | 0.32/0.64 | 20 | 95 | 450 |
| E7 | 3.2 | 18 | 0.34/0.63 | 20 | 95 | 460 |
| V8 | 3.6 | 16 | 0.32/0.64 | 20 | 95 | 570 |
| E8 | 3.2 | 20 | 0.32/0.63 | 20 | 95 | 440 |
| V9 | 3.8 | 16 | 0.32/0.64 | 20 | 95 | 438 |
| E9 | 3.1 | 18 | 0.33/0.63 | 20 | 95 | 430 |
| E10 | 3.3 | 15 | 0.32/0.64 | 20 | 95 | 590 |
| E11 | 3.3 | 20 | 0.32/0.63 | 20 | 95 | 440 |
| E12 | 3.2 | 19 | 0.32/0.64 | 20 | 95 | 487 |
| E13 | 3.4 | 21 | 0.67/0.34 | 20 | 98 | 670 |
| E14 | 3.2 | 19.4 | 0.32/0.63 | 20 | 95 | 510 |
| E15 | 3.4 | 19.1 | 0.32/0.63 | 20 | 95 | 499 |
| V10 | 3.9 | 17.6 | 0.32/0.63 | 20 | 95 | 480 |
| E16 | 3.2 | 19.2 | 0.32/0.64 | 20 | 95 | 491 |
| V11 | 3.4 | 17 | 0.32/0.63 | 20 | 95 | 460 |
| E17 | 3.2 | 19.3 | 0.33/0.64 | 20 | 95 | 489 |
| V12 | 3.5 | 17.1 | 0.32/0.64 | 20 | 95 | 444 |
| E18 | 3.3 | 18.5 | 0.32/0.63 | 20 | 95 | 435 |
| V13 | 3.4 | 17.0 | 0.32/0.64 | 20 | 95 | 487 |
| E19 | 3.1 | 19.4 | 0.32/0.64 | 20 | 95 | 472 |
| | | | CIE x/y | | | |
| | U1000 | EQE | at 1000 | | L₁ | LT |
| Ex. | (V) | 1000 | cd/m² | L₀; j₀ | % | (h) |
| V14 | 5 | 9.0% | 0.13/0.13 | 6000 | 80 | 31 |
| | | | | cd/m² | | |
| V15 | 5.1 | 13% | 0.32/0.63 | 20 | 80 | 41 |
| | | | | mA/cm² | | |
| E20 | 3.6 | 9.0% | 0.13/0.13 | 6000 | 80 | 49 |
| | | | | cd/m² | | |
| E21 | 3.4 | 18% | 0.31/0.63 | 20 | 80 | 150 |
| | | | | mA/cm² | | |
| E22 | 3.3 | 7.3% | 0.14/0.14 | 6000 | 80 | 51 |
| | | | | cd/m² | | |
| E23 | 3.3 | 16.1% | 0.34/0.63 | 20 | 80 | 130 |
| | | | | mA/cm² | | |
| E24 | 3.5 | 15.6% | 0.34/0.63 | 20 | 80 | 170 |
| | | | | mA/cm² | | |
| E25 | 3.3 | 16.3 | 0.34/0.63 | 20 | 80 | 205 |
| | | | | mA/cm² | | |

1.-13. (canceled)

14. A compound of formula (1) 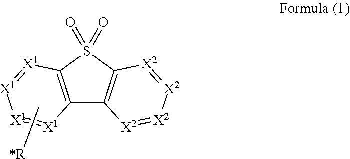
where the symbols used are as follows:
*R is a group of the following formula (2), where the dotted bond represents the bond to the base skeleton in the formula (1): 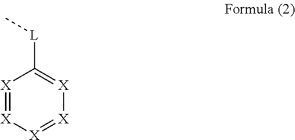
X is the same or different at each instance and is CR^{a} or N, or two adjacent X groups are a group of the following formula (3) or (4), with the proviso that at least one and at most three X groups are N; 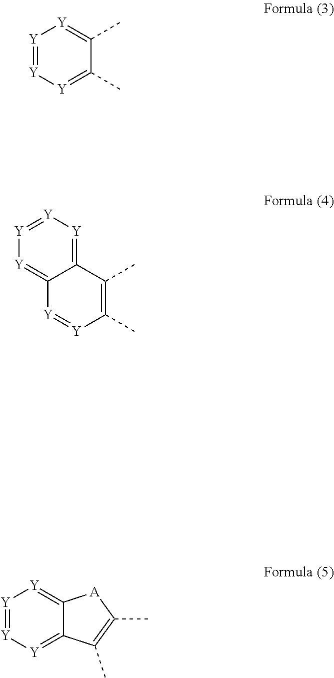
Y is the same or different at each instance and is CR^{a} or N;
A is the same or different at each instance and is NR^{a}, O, S or CR^{a}₂
X¹ is the same or different at each instance and is CR^{b} or N, with the proviso that not more than two X¹ groups are N, and with the further proviso that X¹ is C when the *R group is bonded to that X¹;
X² is the same or different at each instance and is CR^{b} or N, with the proviso that not more than two X² groups are N;
L is a single bond or an aromatic or heteroaromatic ring system which has 5 to 24 aromatic ring atoms and may be substituted by one or more R radicals;
R is the same or different at each instance and is H, D, F, Cl, Br, I, CN, NO₂, N(R¹)₂, C(═O)N(R¹)₂, C(R¹)₃, Si(R¹)₃, B(R¹)₂, C(═O)R¹, P(═O)(R¹)₂, P(R¹)₂, S(═O)R¹, S(═O)₂R¹, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, C(═O)O, C(═O)NR¹, NR¹, P(═O)(R¹), O, S, SO or SO₂, or an aromatic ring system which has 6 to 60 aromatic ring atoms and may be substituted in each case by one or more R¹ radicals, or an electron-rich heteroaryl group which has 5 to 30 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two or more R radicals together may form an aliphatic, heteroaliphatic, aromatic or heteroaromatic ring system which may be substituted by one or more R¹ radicals;
R^{a} is the same or different at each instance and is H, D, F, Cl, Br, I, CN, NO₂, N(R¹)₂, C(═O)N(R¹)₂, C(R¹)₃, Si(R¹)₃, B(R¹)₂, C(═O)R¹, P(═O)(R¹)₂, P(R¹)₂, S(═O)R¹, S(═O)₂R¹, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, C(═O)O, C(═O)NR¹, NR¹, P(═O)(R¹), O, S, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two or more R and/or R^{a} radicals together may form an aliphatic, heteroaliphatic, aromatic or heteroaromatic ring system which may be substituted by one or more R¹ radicals; or at the same time an R or R^{a} radical together with an R^{a} radical may form a ring system which may be substituted by one or more R¹ radicals;
R^{b} is the same or different at each instance and is H, D, F, CN, N(R¹)₂, C(═O)N(R¹)₂, C(R¹)₃, Si(R¹)₃, B(R¹)₂, C(═O)R¹, P(═O)(R¹)₂, P(R¹)₂, S(═O)R¹, S(═O)₂R¹, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, or an aromatic ring system having 6 to 16 aromatic ring atoms, or a heteroaryl group which has 5 to 30 aromatic ring atoms, is joined to the dibenzothiophene dioxide base skeleton via a carbon atom, and may be substituted in each case by one or more R¹ radicals, or an aryloxy or heteroaryloxy group which has 5 to 60 aromatic ring atoms and may be substituted by one or more R¹ radicals; at the same time, two or more R^{b} radicals together may form an aliphatic, heteroaliphatic, aromatic or heteroaromatic ring system which may be substituted by one or more R¹ radicals;
R¹ is the same or different at each instance and is H, D, F, Cl, Br, I, CN, NO₂, N(R²)₂, C(═O)R², P(═O)(R²)₂, P(R²)₂, B(R²)₂, C(R²)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by R²C═CR², C≡C, Si(R²)₂, C═0, C═S, C═Se, C═NR², C(═O)O, C(═O)NR², NR², P(═O)(R²), O, S, SO or SO₂ and where one or more hydrogen atoms may be replaced by D, F, Cl, Br, I, CN or NO₂, or an aromatic ring system which has 6 to 60 aromatic ring atoms and may be substituted in each case by one or more R² radicals, or a heteroaryl group which has 5 to 30 aromatic ring atoms, is bonded to the R, R^{a} or R^{b} group via a carbon atom, and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, where two or more R¹ radicals together may form an aliphatic or heteroaliphatic ring system that may be substituted by one or more R² radicals;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more substituents R² together may form a ring system.

15. A compound as claimed in claim 14, characterized in that X¹ is CR^{b} and/or X² is CR^{b}.

16. A compound as claimed in claim 14, characterized in that the compounds are selected from the formulae (6-1) to (6-4) 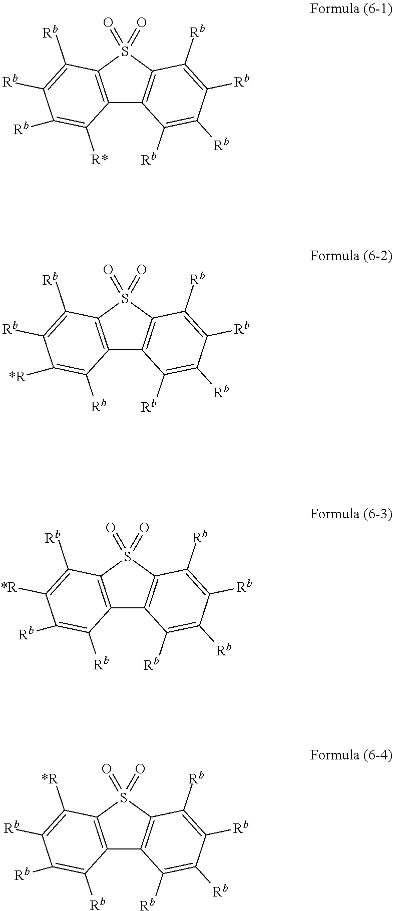
where the symbols used have the definitions given in claim 14, with the proviso that R^{b} is absent when the *R group is bonded to that position.

17. A compound as claimed in claim 14, characterized in that the compounds are selected from the formulae (6-1a) to (6-4f), where the symbols used have the definitions given in claim 14:

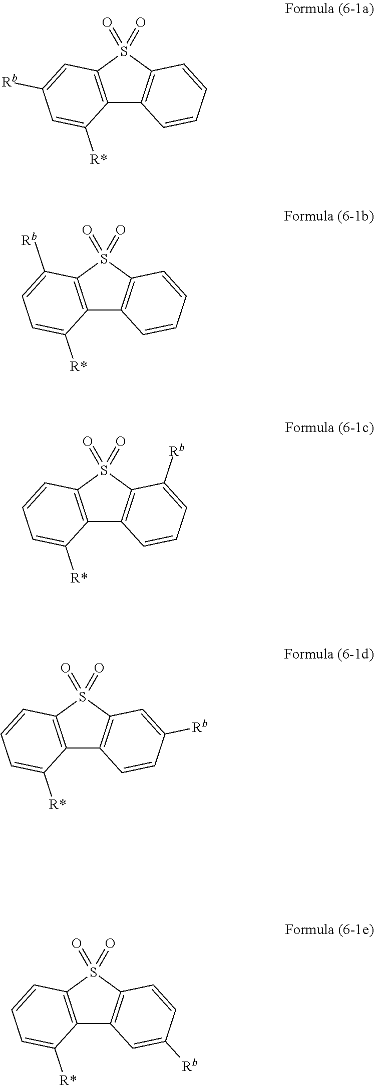

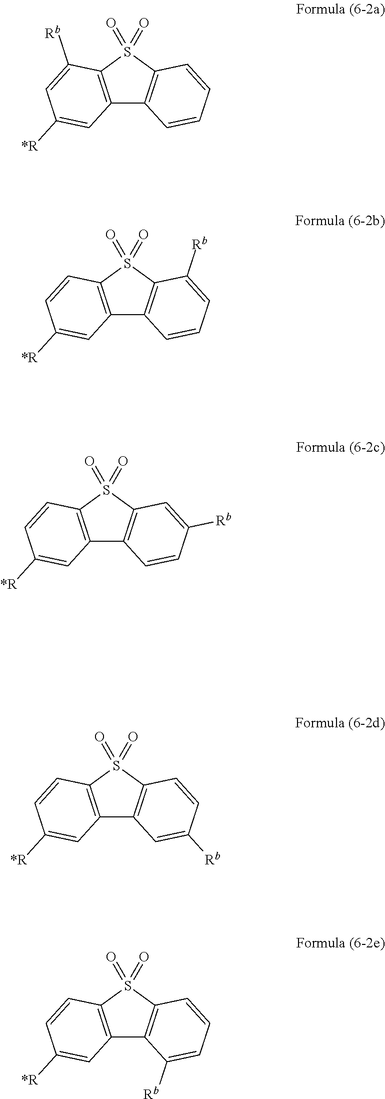

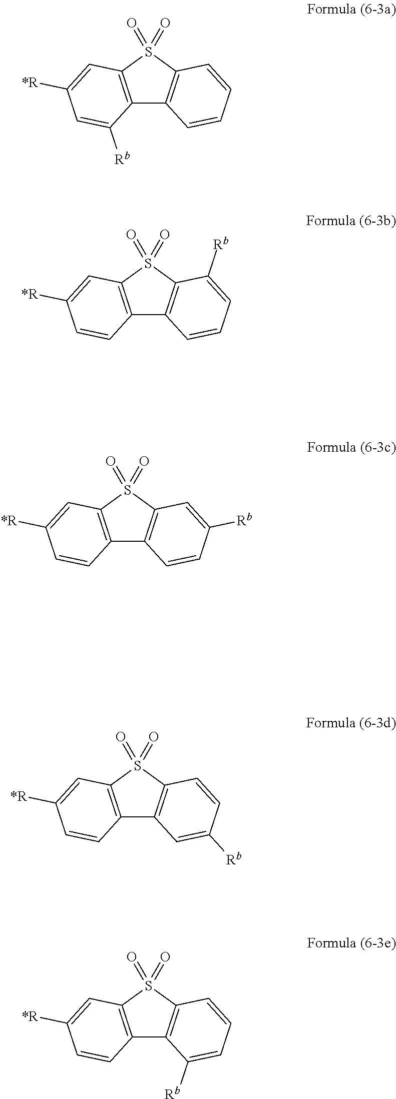

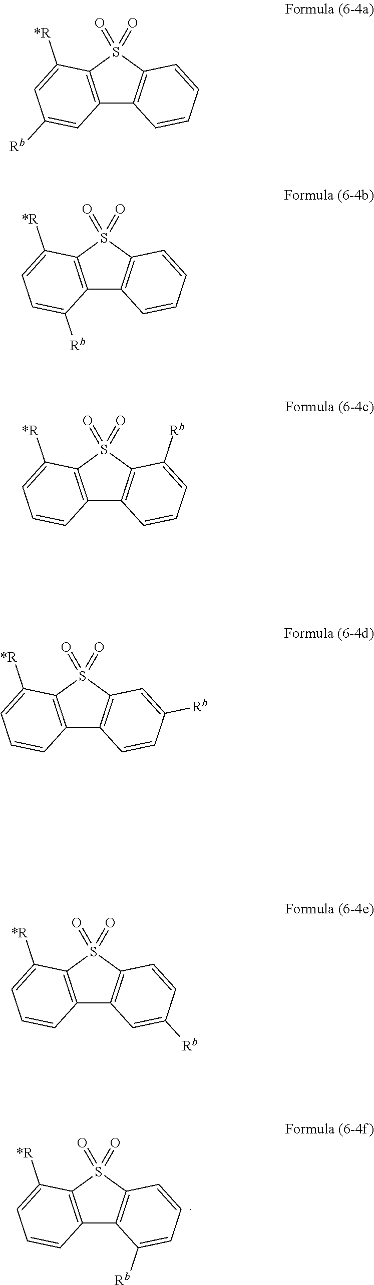

18. A compound as claimed in claim 14, characterized in that L is a single bond or a bivalent aromatic ring system which has 6 to 12 aromatic ring atoms and may be substituted by one or more R radicals, or a dibenzofuran or dibenzothiophene group that may be substituted by one or more R radicals.

19. A compound as claimed in claim 14, characterized in that L, when L is an aromatic or heteroaromatic ring system, is selected from the structures of the formulae (L-1) to (L-34) 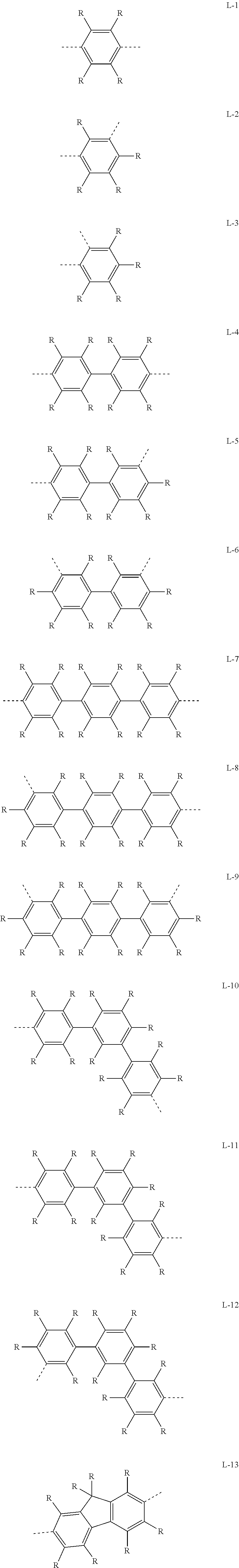 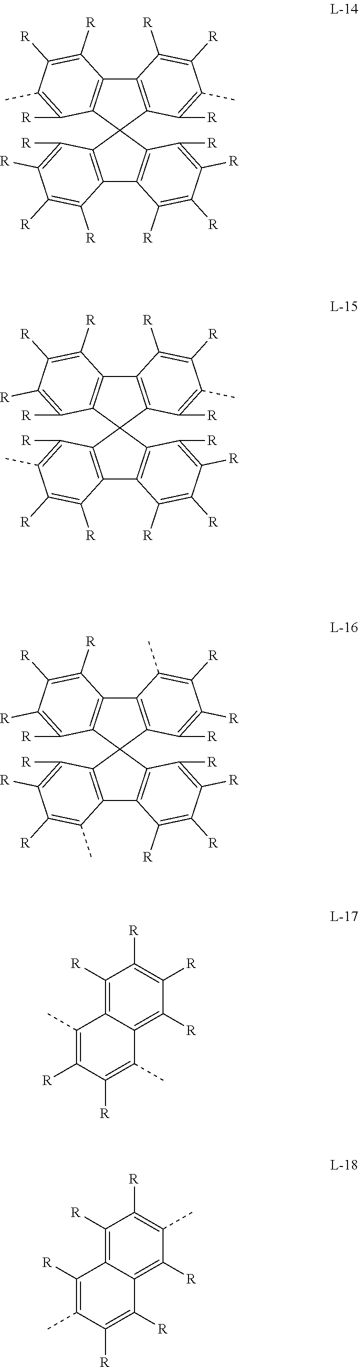 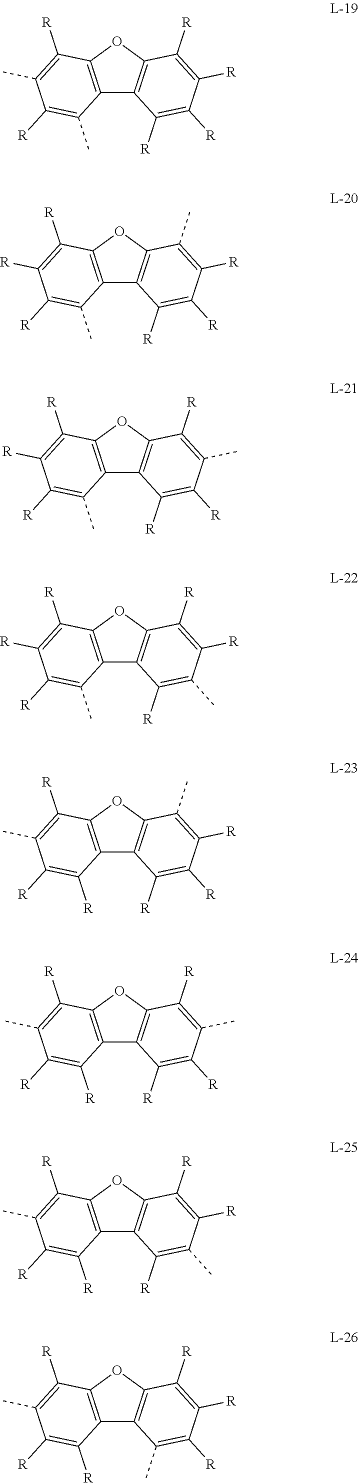 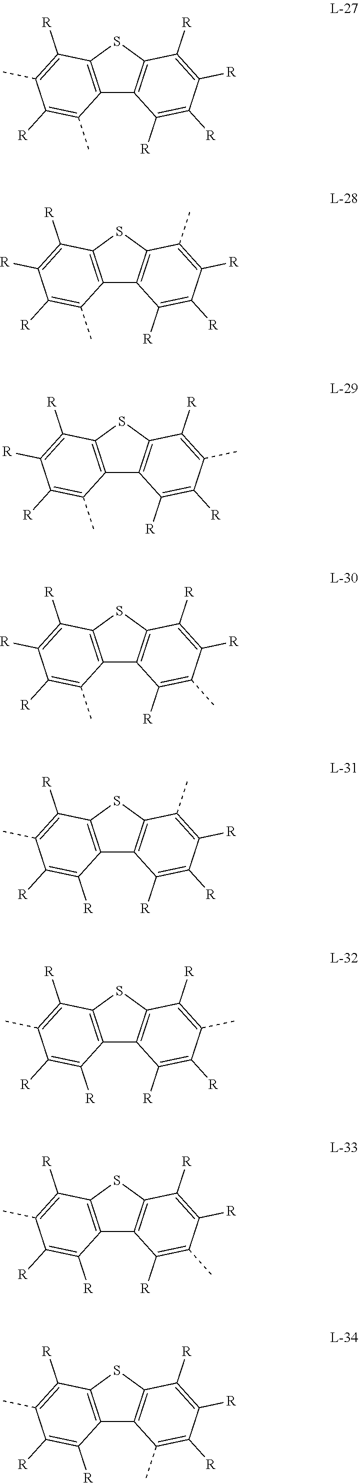
where the symbols have the meanings given in claim 14 and the dotted bonds represent the bonds to the heteroaryl group in the group of the formula (2) and to the base skeleton of the compound of the formula (1).

20. A compound as claimed in claim 14, characterized in that the group of the formula (2) is selected from the structures of the formulae (7) to (21): 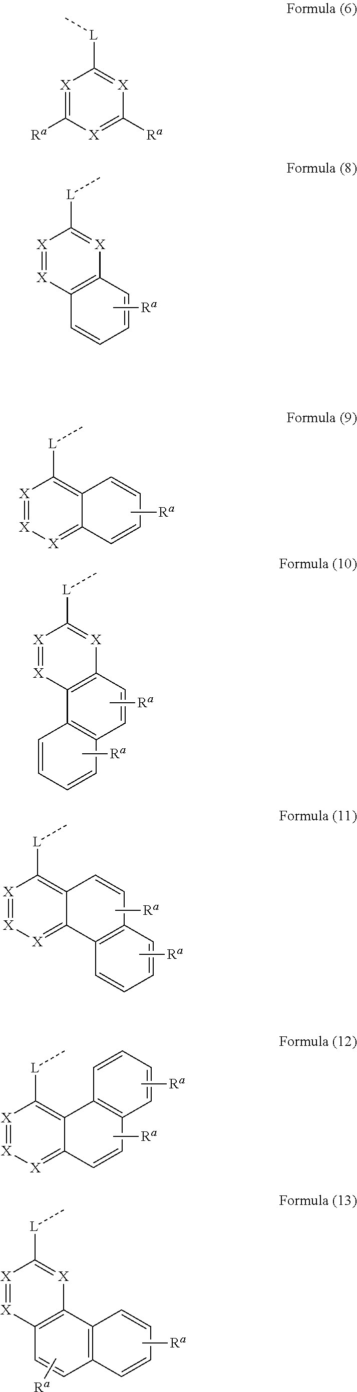 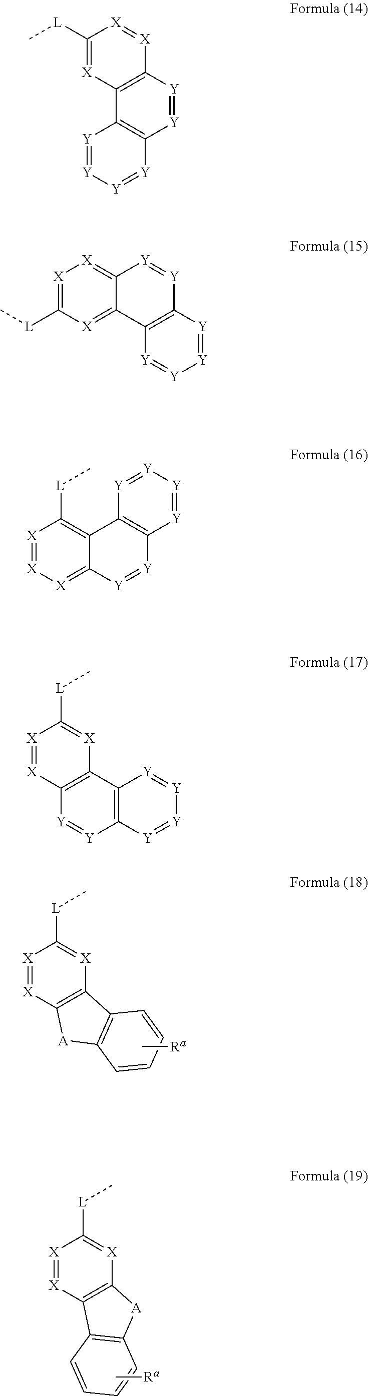 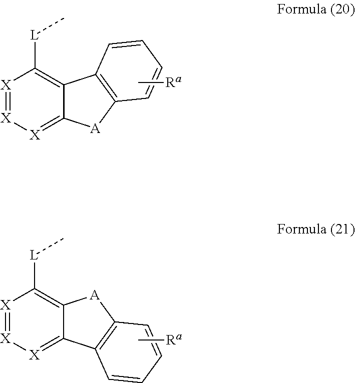
where the symbols used have the definitions given in claim 14 and, in formula (7), one, two or three X are N and R^{a} is the same or different at each instance and is H, D or an aromatic or heteroaromatic ring system which has 5 to 40 aromatic ring atoms and may be substituted by one or more R¹ radicals, and where, in formulae (8) to (13), exactly two X are N and R^{a} may also occur more than once, and where, in the formulae (14) to (17), exactly one X group is N and exactly one Y group is N, and where, in the formulae (18) to (21), exactly two X are N and R^{a} may also occur repeatedly.

21. A compound as claimed in claim 14, characterized in that the group of the formula (2) is selected from the groups of the formulae (7a) to (21a): 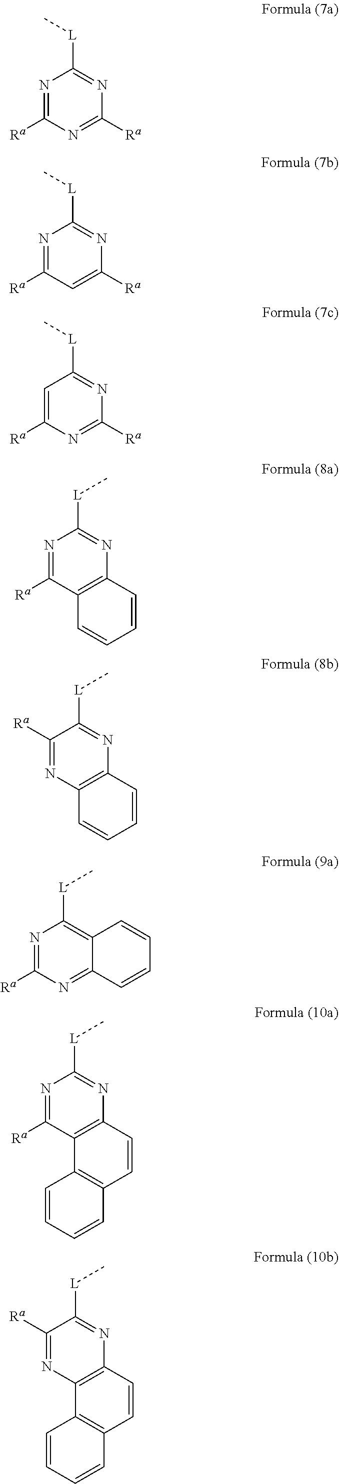 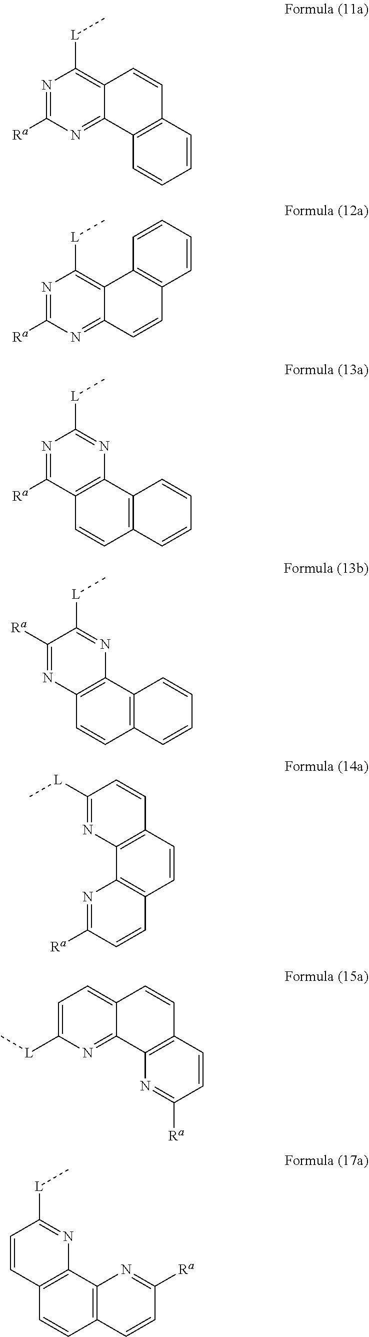 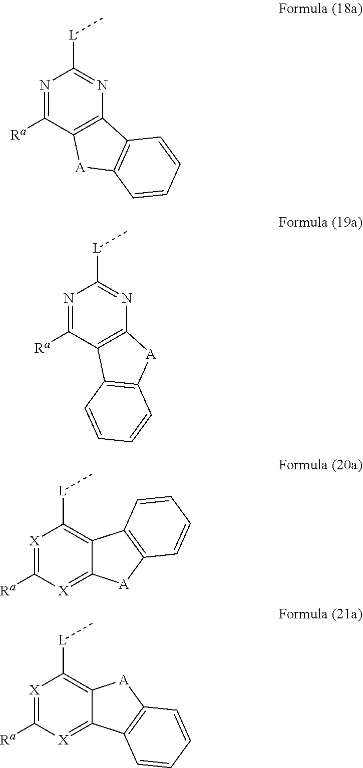
where the symbols have the definitions given in claim 14 and R^{a} is the same or different at each instance and is H, D or an aromatic or heteroaromatic ring system which has 5 to 40 aromatic ring atoms and may be substituted by one or more R¹ radicals.

22. A process for preparing a compound as claimed in claim 14, characterized by the steps of:
(a) synthesizing the dibenzothiophene dioxide base skeleton that bears, in place of the substituent *R, a reactive leaving group, and
(b) introducing the substituent *R by a coupling reaction.

23. A formulation comprising at least one compound as claimed in claim 14 and at least one further compound and/or at least one solvent.

24. The use of a compound as claimed in claim 14 in an electronic device.

25. An electronic device comprising at least one compound as claimed in claim 14, wherein the electronic device is preferably selected from the group consisting of organic electroluminescent devices, organic integrated circuits, organic field-effect transistors, organic thin-film transistors, organic light-emitting transistors, organic solar cells, organic optical detectors, organic photoreceptors, organic field-quench devices, light-emitting electrochemical cells and organic laser diodes.

26. The electronic device as claimed in claim 25, which is an organic electroluminescent device, characterized in that the device comprises anode, cathode and at least one emitting layer, where at least one organic layer that may be an emitting layer, hole transport layer, electron transport layer, hole blocker layer, electron blocker layer or other functional layer comprises the at least one compound.
